(19) United States
(12) Patent Application Publication
(10) Pub. No.: US 2025/0324907 A1
(43) Pub. Date: Oct. 16, 2025

(54) SPIRO COMPOUND AND USE THEREOF

(71) Applicant: SICHUAN AG-RAY NEW MATERIALS CO., LTD., Meishan City, Sichuan (CN)

(72) Inventors: Liangliang YAN, Foshan (CN); Shaofu CHEN, Foshan (CN); Lei DAI, Foshan (CN); Lifei CAI, Foshan (CN)

(73) Assignee: SICHUAN AG-RAY NEW MATERIALS CO., LTD., Meishan City, Sichuan (CN)

(21) Appl. No.: 18/693,674

(22) PCT Filed: Aug. 30, 2022
(86) PCT No.: PCT/CN2022/115659

§ 371 Date: Nov. 20, 2024

(30) Foreign Application Priority Data
Sep. 28, 2021 (CN) 202111141918.4
Jul. 19, 2022 (CN) 202210844413.2

(51) Int. Cl. H10K 85/60; C07D 471/04; C09K 11/06; H10K 50/16; H10K 50/17

(52) U.S. Cl. CPC H10K 85/624; C07D 471/04; C09K 11/06; H10K 85/626; H10K 85/654; H10K 85/657; H10K 85/6572; H10K 85/6574; C09K 2211/1018; H10K 50/16; H10K 50/171

(57) ABSTRACT

The present invention relates to a spiro compound and application thereof. The spiro compound has a structure as shown in a formula (1). The material provided in the present invention has the advantages of high optical and electrical stability, low sublimation temperature, low driving voltage, high luminous efficiency, long device service life and the like, and can be used in an organic electroluminescent device. In particular, the compound has the possibility of being applied in the AMOLED industry as an electron injection or electron transport material.

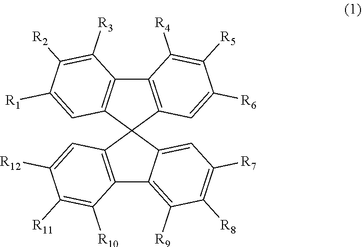

### TECHNICAL FIELD

The present invention relates to the technical field of organic electroluminescence, in particular to an organic light-emitting material applicable to organic electroluminescent devices, and specially in particular to a spiro compound and application thereof.

### BACKGROUND

At present, as a new-generation display technology, an organic electroluminescent device (OLED) has attracted more and more attention in display and lighting technologies, thus having a wide application prospect. However, compared with market application requirements, properties, such as luminous efficiency, driving voltage and service life, of the OLED still need to be strengthened and improved.

In generally, the OLED includes various organic functional material films with different functions sandwiched between metal electrodes as a basic structure, which is similar to a sandwich structure. Under the driving of a current, holes and electrons are injected from a cathode and an anode, respectively. After moving to a certain distance, the holes and the electrons are compounded in a light-emitting layer, and then released in the form of light or heat to achieve luminescence of the OLED. However, organic functional materials are core components of the OLED, and the thermal stability, photochemical stability, electrochemical stability, quantum yield, film forming stability, crystallinity, color saturation and the like of the materials are main factors affecting properties of the device.

In order to obtain organic light-emitting devices with excellent properties, the selection of materials is particularly important. Not only is an emitter material having a light-emitting effect included, but also a hole injection material, a hole transport material, a main material, an electron transport material, an electron injection material and other functional materials that are mainly used for injection and transportation of carriers in the devices are included. Through selection and optimization of the materials, the transportation efficiency of holes and electrons can be improved, and the holes and the electrons in the devices can reach a balance, so that the voltage, luminous efficiency, and service life of the devices are improved.

A patent document (CN106536485) records a structure obtained by connecting triazine and benzimidazole on one side or two sides of spirofluorene simultaneously to serve as an electron transport material, and discloses a compound

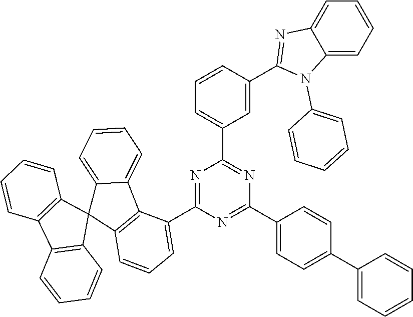 [SMILES: c1ccc(-c2ccc(-c3nc(-c4cccc(-c5nc6ccccc6n5-c5ccccc5)c4)nc(-c4cccc5c4-c4ccccc4C54c5ccccc5-c5ccccc54)n3)cc2)cc1]

as an electron transport layer (ETL), but the driving voltage and device service life of such material need to be improved. A patent document (CN108602783) discloses a spirofluorene structure

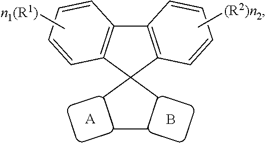

in which A or B is a naphthalene or phenanthrene structure, and discloses a compound

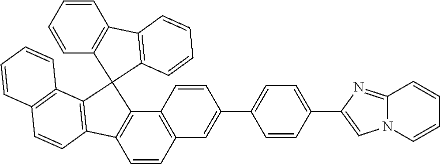 [SMILES: c1ccc2c(c1)-c1ccccc1C21c2c(ccc3ccccc23)-c2ccc3cc(-c4ccc(-c5cn6ccccc6n5)cc4)ccc3c21]

as a blue emitter or an electron transport material, but the driving voltage and device service life of such material also need to be improved. A patent document (CN110804053A) discloses an imidazo-N heterocyclic ring structure

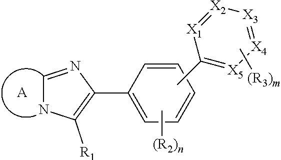

as an electronic transport material, but the device properties, especially the service life, of such material need to be improved. A patent document (CN111925366A) discloses an imidazopyridine structure

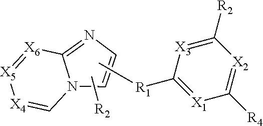

as an electron transport material or a hole blocking layer material or a capping layer material, but the device properties, especially the device voltage, of such material need to be improved. A patent document (CN104650116A) and a patent document (CN104650117A) disclose an imidazo-N heterocyclic ring structure

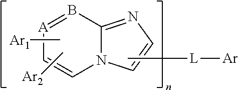

as an electronic transport material, but the device voltage of such material is relatively high and needs to be further improved.

### SUMMARY

In order to overcome the above defects, the present invention provides an organic electroluminescent device with high properties and a spiro compound material capable of realizing the organic electroluminescent device.

The spiro compound of the present invention has a structure as shown in a formula (1). The spiro compound provided in the present invention has the advantages of high optical and electrical stability, low sublimation temperature, low driving voltage, high luminous efficiency, long device service life and the like, and can be used in an organic electroluminescent device. In particular, the compound has the possibility of being applied to the AMOLED industry as an electron injection or transport material.

A spiro compound has a structure as shown in a formula (1),

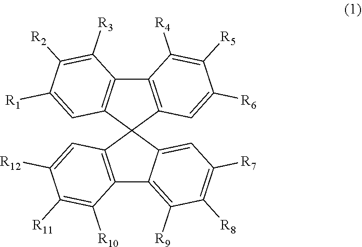

where R₁-R₁₂ are independently selected from hydrogen, deuterium, halogen, cyano, hydroxyl, sulfhydryl, amino, substituted or unsubstituted C₁-C₁₀ alkyl, substituted or unsubstituted C₁-C₁₀heteroalkyl, substituted or unsubstituted C₃-C₂₀ cycloalkyl, substituted or unsubstituted C₃-C₂₀heterocycloalkyl, substituted or unsubstituted C₂-C₁₀ alkenyl, substituted or unsubstituted C₂-C₁₀ alkynyl, substituted or unsubstituted C₆-C₃₀ aryl, substituted or unsubstituted C₂-C₃₀ heteroaryl, substituted or unsubstituted tri-C₁-C₁₀alkylsilyl, substituted or unsubstituted tri-C₆-C₁₂arylsilyl, substituted or unsubstituted di-C₁-C₁₀ alkyl mono-C₆-C₃₀arylsilyl, and substituted or unsubstituted mono-C₁-C₁₀ alkyl di-C₆-C₃₀arylsilyl, or two adjacent groups are connected to each other to form an aliphatic ring, a heteroaromatic ring or an aromatic ring structure; and
at least one of the R₁-R₁₂ has a structure as shown in a formula (2),

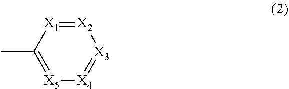

where in the formula (2),
X₁-X₅ are independently selected from N or CR₀; in the X₁-X₅, at least one is N, and at least one is CR₀;
R₀ is independently selected from hydrogen, deuterium, halogen, cyano, hydroxyl, sulfhydryl, amino, substituted or unsubstituted C₁-C₁₀ alkyl, substituted or unsubstituted C₁-C₁₀heteroalkyl, substituted or unsubstituted C₃-C₂₀ cycloalkyl, substituted or unsubstituted C₃-C₂₀heterocycloalkyl, substituted or unsubstituted C₂-C₁₀ alkenyl, substituted or unsubstituted C₂-C₁₀ alkynyl, substituted or unsubstituted C₆-C₃₀ aryl, substituted or unsubstituted C₂-C₃₀ heteroaryl, substituted or unsubstituted tri-C₁-C₁₀alkylsilyl, substituted or unsubstituted tri-C₆-C₁₂arylsilyl, substituted or unsubstituted di-C₁-C₁₀ alkyl mono-C₆-C₃₀arylsilyl, and substituted or unsubstituted mono-C₁-C₁₀ alkyl di-C₆-C₃₀arylsilyl, or two adjacent groups are connected to each other to form an aliphatic ring, a heteroaromatic ring or an aromatic ring structure; and
at least one of the R₀ has a structure as shown in a formula (3) or a formula (4),

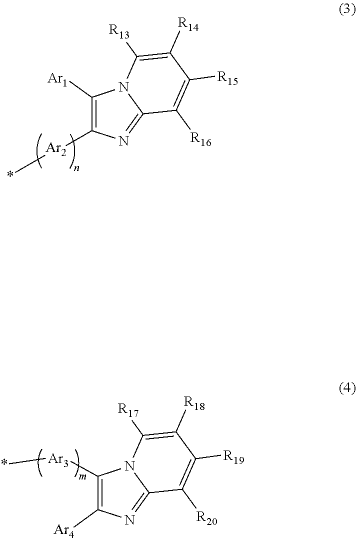

where * indicates a position connected with the formula (2);
m and n are independently selected from an integer of 0-4, and when the n or the m is 0, the formula (3) or the formula (4) is directly connected with the formula (2);
R₁₃-R₂₀ are independently selected from hydrogen, deuterium, halogen, cyano, hydroxyl, sulfhydryl, amino, substituted or unsubstituted C₁-C₁₀ alkyl, substituted or unsubstituted C₁-C₁₀heteroalkyl, substituted or unsubstituted C₃-C₂₀ cycloalkyl, substituted or unsubstituted C₃-C₂₀heterocycloalkyl, substituted or unsubstituted C₂-C₁₀ alkenyl, substituted or unsubstituted C₂-C₁₀ alkynyl, substituted or unsubstituted C₆-C₃₀ aryl, substituted or unsubstituted C₂-C₃₀ heteroaryl, substituted or unsubstituted tri-C₁-C₁₀alkylsilyl, substituted or unsubstituted tri-C₆-C₁₂arylsilyl, substituted or unsubstituted di-C₁-C₁₀ alkyl mono-C₆-C₃₀arylsilyl, and substituted or unsubstituted mono-C₁-C₁₀ alkyl di-C₆-C₃₀arylsilyl, or two adjacent groups are connected to each other to form an aliphatic ring, a heteroaromatic ring or an aromatic ring structure;
Ar₁-Ar₄ are independently selected from substituted or unsubstituted C₆-C₃₀ aryl, or substituted or unsubstituted C₄-C₃₀ heteroaryl;
the heteroalkyl, the heterocycloalkyl, the heteroaromatic ring and the heteroaryl at least contain one O, N, or S heteroatom;
and the “substituted” refers to substitution with deuterium, F, Cl, Br, C₁-C₆ alkyl, C₃-C₆ cycloalkyl, C₁-C₆ alkylamino, C₆-C₁₀ aryl, cyano, isocyano, or phosphino, and the substitution ranges from a single substitution number to a maximum substitution number.

A optional spiro compound has a structure as shown in a formula (5) or a formula (6),

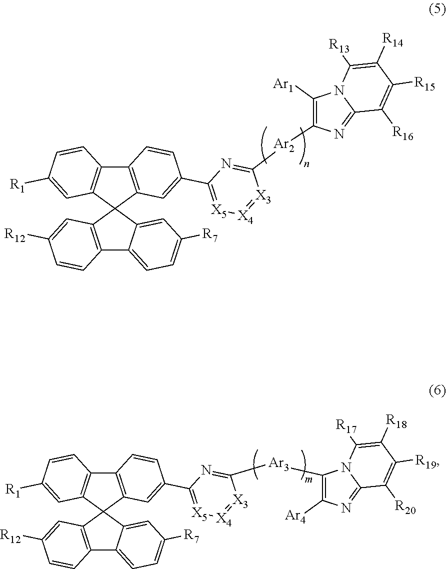

where R₁, R₇, R₁₂ and R₁₃-R₂₀ are independently selected from hydrogen, deuterium, halogen, cyano, hydroxyl, sulfhydryl, amino, substituted or unsubstituted C₁-C₁₀ alkyl, substituted or unsubstituted C₁-C₁₀heteroalkyl, substituted or unsubstituted C₃-C₂₀ cycloalkyl, substituted or unsubstituted C₃-C₂₀heterocycloalkyl, substituted or unsubstituted C₂-C₁₀ alkenyl, substituted or unsubstituted C₂-C₁₀ alkynyl, substituted or unsubstituted C₆-C₃₀ aryl, substituted or unsubstituted C₂-C₃₀ heteroaryl, substituted or unsubstituted tri-C₁-C₁₀alkylsilyl, substituted or unsubstituted tri-C₆-C₁₂arylsilyl, substituted or unsubstituted di-C₁-C₁₀ alkyl mono-C₆-C₃₀arylsilyl, and substituted or unsubstituted mono-C₁-C₁₀ alkyl di-C₆-C₃₀arylsilyl, or two adjacent groups are connected to each other to form an aliphatic ring, a heteroaromatic ring or an aromatic ring structure;
X₃-X₅ are independently selected from N or CR₀;
R₀ is independently selected from hydrogen, deuterium, halogen, cyano, hydroxyl, sulfhydryl, amino, substituted or unsubstituted C₁-C₁₀ alkyl, substituted or unsubstituted C₁-C₁₀heteroalkyl, substituted or unsubstituted C₃-C₂₀ cycloalkyl, substituted or unsubstituted C₃-C₂₀heterocycloalkyl, substituted or unsubstituted C₂-C₁₀ alkenyl, substituted or unsubstituted C₂-C₁₀ alkynyl, substituted or unsubstituted C₆-C₃₀ aryl, substituted or unsubstituted C₂-C₃₀ heteroaryl, substituted or unsubstituted tri-C₁-C₁₀alkylsilyl, substituted or unsubstituted tri-C₆-C₁₂arylsilyl, substituted or unsubstituted di-C₁-C₁₀ alkyl mono-C₆-C₃₀arylsilyl, and substituted or unsubstituted mono-C₁-C₁₀ alkyl di-C₆-C₃₀arylsilyl, or two adjacent groups may be connected to each other to form an aliphatic ring, a heteroaromatic ring or an aromatic ring structure;
m and n are independently selected from an integer of 0-4;
Ar₁-Ar₄ are independently selected from substituted or unsubstituted C₆-C₃₀ aryl, or substituted or unsubstituted C₄-C₃₀ heteroaryl;
the heteroalkyl, the heterocycloalkyl, the heteroaromatic ring structure and the heteroaryl at least contain one O, N, or S heteroatom;
and the “substituted” refers to substitution with deuterium, F, Cl, Br, C₁-C₆ alkyl, C₃-C₆ cycloalkyl, C₁-C₆ alkylamino, C₆-C₁₀ aryl, cyano, isocyano, or phosphino, and the substitution ranges from a single substitution number to a maximum substitution number.

The R₁, the R₇, the R₁₂ and the R₁₃-R₂₀ are independently selected from hydrogen, deuterium, halogen, cyano, substituted or unsubstituted C₁-C₁₀ alkyl, substituted or unsubstituted C₃-C₂₀ cycloalkyl, substituted or unsubstituted C₆-C₃₀ aryl, and substituted or unsubstituted C₂-C₃₀ heteroaryl, or two adjacent groups are connected to each other to form a heteroaromatic ring or an aromatic ring structure;
the X₃-X₅ are independently selected from N or CR₀;
the R₀ is independently selected from hydrogen, deuterium, halogen, cyano, substituted or unsubstituted C₁-C₁₀ alkyl, substituted or unsubstituted C₃-C₂₀ cycloalkyl, substituted or unsubstituted C₆-C₃₀ aryl, and substituted or unsubstituted C₂-C₃₀ heteroaryl, or two adjacent groups are connected to each other to form a heteroaromatic ring or an aromatic ring structure;
the m and the n are independently selected from an integer of 0-2;
the Ar₁-Ar₄ are independently selected from substituted or unsubstituted C₆-C₃₀ aryl, or substituted or unsubstituted C₄-C₃₀ heteroaryl;
the heteroaromatic ring structure and the heteroaryl at least contain one O or N heteroatom;
and the “substituted” refers to substitution with deuterium, F, Cl, Br, C₁-C₆ alkyl, C₃-C₆ cycloalkyl, or C₆-C₁₀ aryl, and the substitution ranges from a single substitution number to a maximum substitution number.

Optionally, in the X₃-X₅, at least one is N.

Further optionally, in the X₃-X₅, the X₃ and/or the X₅ is N.

When the X₃ and/or the X₅ is N, the X₄ is CR₀, and the R₀ is not H.

When the X₃ and the X₅ are N, the X₄ is CR₀, and the R₀ is substituted or unsubstituted C₆-C₃₀ aryl, or substituted or unsubstituted C₂-C₃₀ heteroaryl.

When the X₅ is N and the X₄ and the X₃ are CR₀, one of two R₀ is substituted or unsubstituted C₆-C₂₀ aryl, or substituted or unsubstituted C₂-C₂₀ heteroaryl, and the other one is hydrogen, or the two R₀ are connected to each other to form an aromatic ring or a heteroaromatic ring; and when the X₃ is N and the X₄ and the X₅ are CR₀, one of two R₀ is substituted or unsubstituted C₆-C₂₀ aryl, or substituted or unsubstituted C₂-C₂₀ heteroaryl, and the other one is hydrogen, or the two R₀ are connected to each other to form an aromatic ring or a heteroaromatic ring.

The m or the n is an integer of 1-2.

The Ar₁-Ar₄ are independently selected from substituted or unsubstituted C₆-C₁₈ aromatic ring, or substituted or unsubstituted C₄-C₁₈ heteroaryl.

The Ar₁-Ar₄ are independently selected from substituted or unsubstituted C₆-C₁₀ aromatic ring.

At least one of the R₁, the R₇ and the R₁₂ is F or CN, and the other groups are hydrogen.

The R₁, the R₇ and the R₁₂ are hydrogen.

At least one group of two adjacent substituents in the R₁₃-R₁₆ and the R₁₇-R₂₀ are connected to each other to form an aromatic ring or a heteroaromatic ring, and the other groups are hydrogen.

The R₁₃-R₁₆ and the R₁₇-R₂₀ are hydrogen.

As a optional spiro compound, the spiro compound has one of the following structural formulas, or is partially or completely deuterated or fluorinated correspondingly,

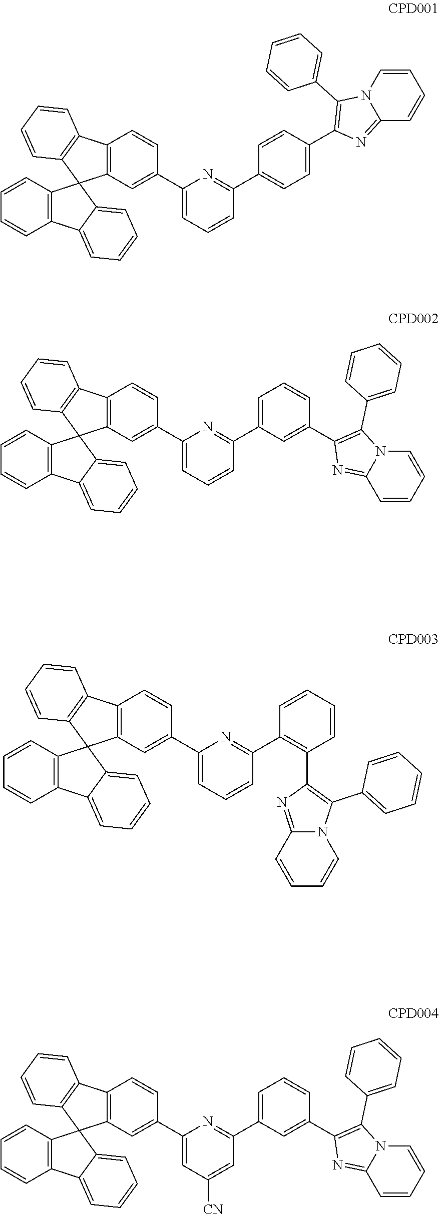 [SMILES: c1ccc(-c2c(-c3ccc(-c4cccc(-c5ccc6c(c5)C5(c7ccccc7-c7ccccc75)c5ccccc5-6)n4)cc3)nc3ccccn23)cc1 ; c1ccc(-c2c(-c3cccc(-c4cccc(-c5ccc6c(c5)C5(c7ccccc7-c7ccccc75)c5ccccc5-6)n4)c3)nc3ccccn23)cc1 ; c1ccc(-c2c(-c3ccccc3-c3cccc(-c4ccc5c(c4)C4(c6ccccc6-c6ccccc64)c4ccccc4-5)n3)nc3ccccn23)cc1 ; N#Cc1cc(-c2cccc(-c3nc4ccccn4c3-c3ccccc3)c2)nc(-c2ccc3c(c2)C2(c4ccccc4-c4ccccc42)c2ccccc2-3)c1]

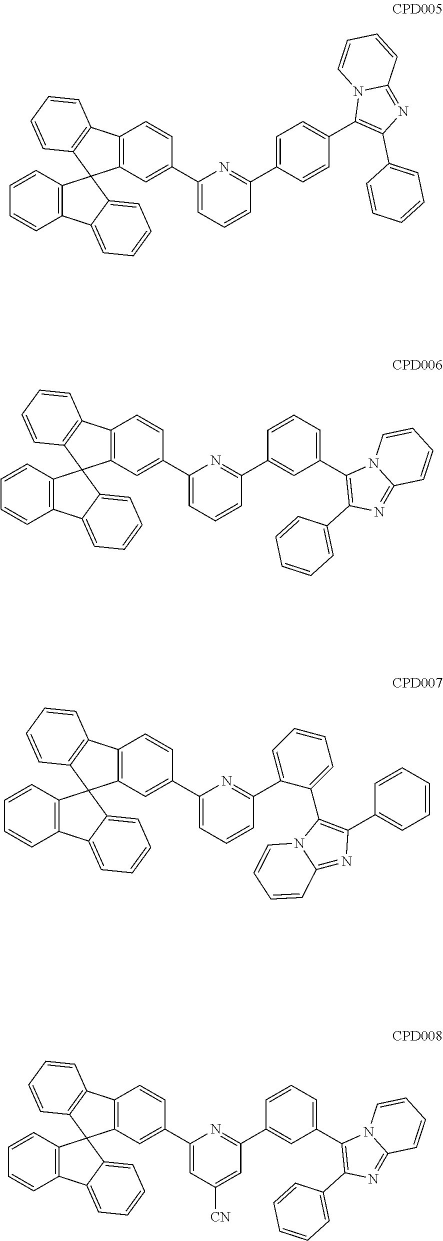 [SMILES: c1ccc(-c2nc3ccccn3c2-c2ccc(-c3cccc(-c4ccc5c(c4)C4(c6ccccc6-c6ccccc64)c4ccccc4-5)n3)cc2)cc1 ; c1ccc(-c2nc3ccccn3c2-c2cccc(-c3cccc(-c4ccc5c(c4)C4(c6ccccc6-c6ccccc64)c4ccccc4-5)n3)c2)cc1 ; c1ccc(-c2nc3ccccn3c2-c2ccccc2-c2cccc(-c3ccc4c(c3)C3(c5ccccc5-c5ccccc53)c3ccccc3-4)n2)cc1 ; N#Cc1cc(-c2cccc(-c3c(-c4ccccc4)nc4ccccn34)c2)nc(-c2ccc3c(c2)C2(c4ccccc4-c4ccccc42)c2ccccc2-3)c1]

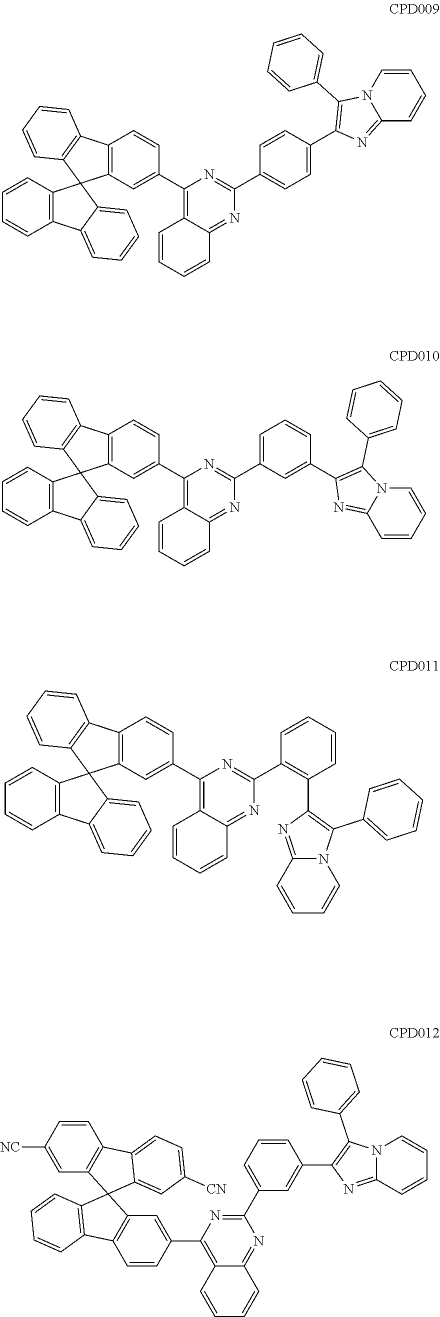 [SMILES: c1ccc(-c2c(-c3ccc(-c4nc(-c5ccc6c(c5)C5(c7ccccc7-c7ccccc75)c5ccccc5-6)c5ccccc5n4)cc3)nc3ccccn23)cc1 ; c1ccc(-c2c(-c3cccc(-c4nc(-c5ccc6c(c5)C5(c7ccccc7-c7ccccc75)c5ccccc5-6)c5ccccc5n4)c3)nc3ccccn23)cc1 ; c1ccc(-c2c(-c3ccccc3-c3nc(-c4ccc5c(c4)C4(c6ccccc6-c6ccccc64)c4ccccc4-5)c4ccccc4n3)nc3ccccn23)cc1 ; N#Cc1ccc2c(c1)C1(c3ccccc3-c3ccc(-c4nc(-c5cccc(-c6nc7ccccn7c6-c6ccccc6)c5)nc5ccccc45)cc31)c1cc(C#N)ccc1-2]

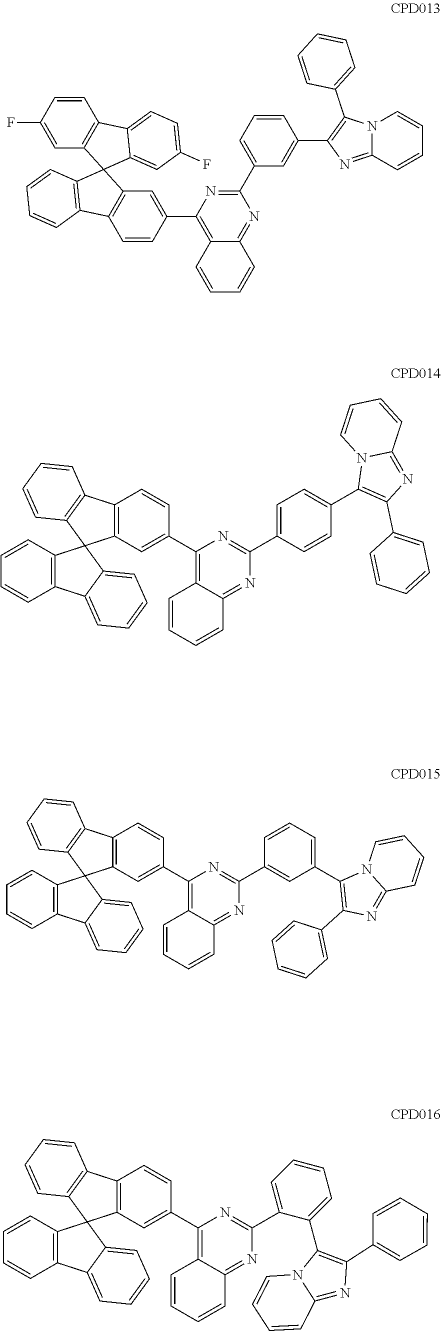 [SMILES: Fc1ccc2c(c1)C1(c3ccccc3-c3ccc(-c4nc(-c5cccc(-c6nc7ccccn7c6-c6ccccc6)c5)nc5ccccc45)cc31)c1cc(F)ccc1-2 ; c1ccc(-c2nc3ccccn3c2-c2ccc(-c3nc(-c4ccc5c(c4)C4(c6ccccc6-c6ccccc64)c4ccccc4-5)c4ccccc4n3)cc2)cc1 ; c1ccc(-c2nc3ccccn3c2-c2cccc(-c3nc(-c4ccc5c(c4)C4(c6ccccc6-c6ccccc64)c4ccccc4-5)c4ccccc4n3)c2)cc1 ; c1ccc(-c2nc3ccccn3c2-c2ccccc2-c2nc(-c3ccc4c(c3)C3(c5ccccc5-c5ccccc53)c3ccccc3-4)c3ccccc3n2)cc1]

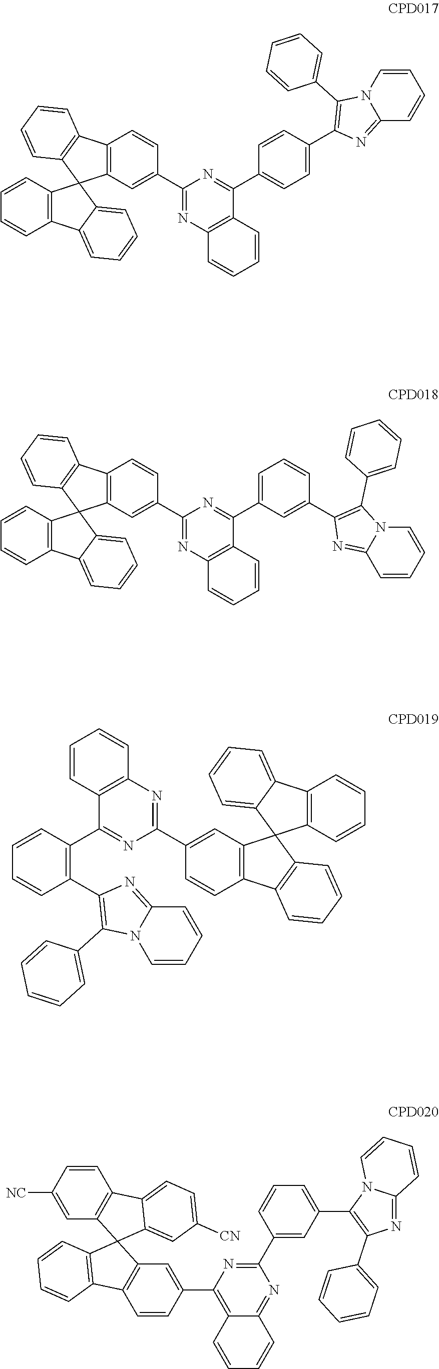 [SMILES: c1ccc(-c2c(-c3ccc(-c4nc(-c5ccc6c(c5)C5(c7ccccc7-c7ccccc75)c5ccccc5-6)nc5ccccc45)cc3)nc3ccccn23)cc1 ; c1ccc(-c2c(-c3cccc(-c4nc(-c5ccc6c(c5)C5(c7ccccc7-c7ccccc75)c5ccccc5-6)nc5ccccc45)c3)nc3ccccn23)cc1 ; c1ccc(-c2c(-c3ccccc3-c3nc(-c4ccc5c(c4)C4(c6ccccc6-c6ccccc64)c4ccccc4-5)nc4ccccc34)nc3ccccn23)cc1 ; N#Cc1ccc2c(c1)C1(c3ccccc3-c3ccc(-c4nc(-c5cccc(-c6c(-c7ccccc7)nc7ccccn67)c5)nc5ccccc45)cc31)c1cc(C#N)ccc1-2]

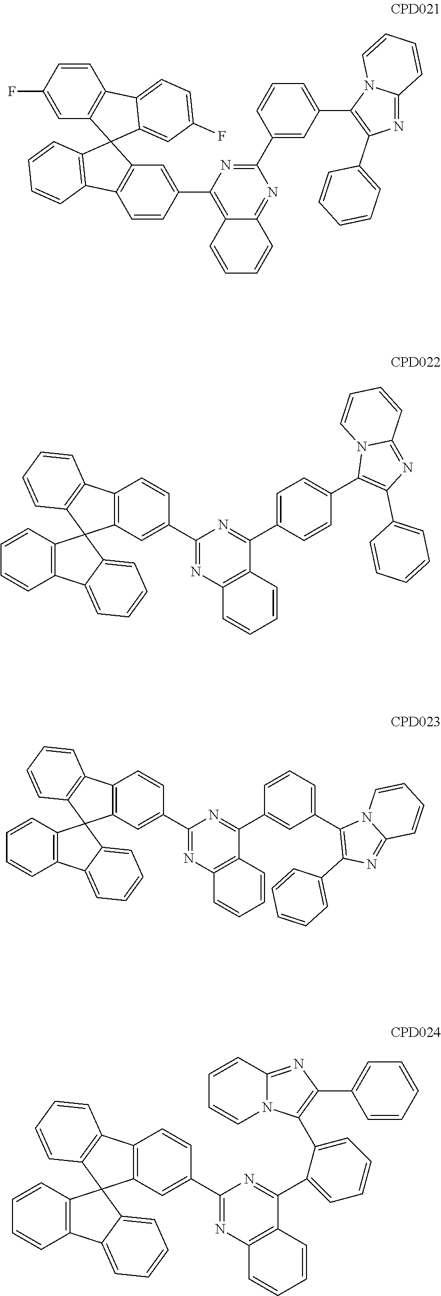 [SMILES: Fc1ccc2c(c1)C1(c3ccccc3-c3ccc(-c4nc(-c5cccc(-c6c(-c7ccccc7)nc7ccccn67)c5)nc5ccccc45)cc31)c1cc(F)ccc1-2 ; c1ccc(-c2nc3ccccn3c2-c2ccc(-c3nc(-c4ccc5c(c4)C4(c6ccccc6-c6ccccc64)c4ccccc4-5)nc4ccccc34)cc2)cc1 ; c1ccc(-c2nc3ccccn3c2-c2cccc(-c3nc(-c4ccc5c(c4)C4(c6ccccc6-c6ccccc64)c4ccccc4-5)nc4ccccc34)c2)cc1 ; c1ccc(-c2nc3ccccn3c2-c2ccccc2-c2nc(-c3ccc4c(c3)C3(c5ccccc5-c5ccccc53)c3ccccc3-4)nc3ccccc23)cc1]

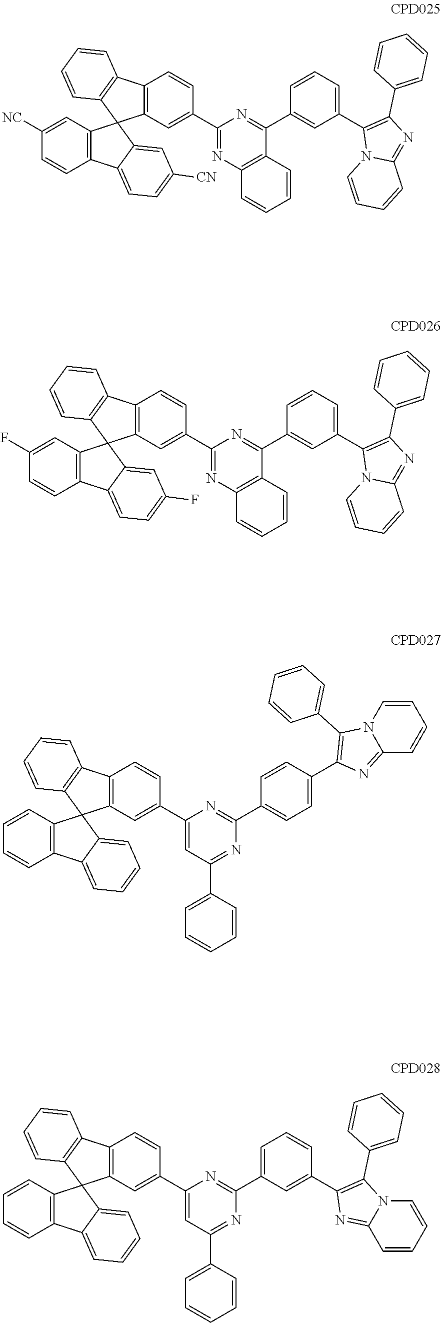 [SMILES: N#Cc1ccc2c(c1)C1(c3ccccc3-c3ccc(-c4nc(-c5cccc(-c6c(-c7ccccc7)nc7ccccn67)c5)c5ccccc5n4)cc31)c1cc(C#N)ccc1-2 ; Fc1ccc2c(c1)C1(c3ccccc3-c3ccc(-c4nc(-c5cccc(-c6c(-c7ccccc7)nc7ccccn67)c5)c5ccccc5n4)cc31)c1cc(F)ccc1-2 ; c1ccc(-c2cc(-c3ccc4c(c3)C3(c5ccccc5-c5ccccc53)c3ccccc3-4)nc(-c3ccc(-c4nc5ccccn5c4-c4ccccc4)cc3)n2)cc1 ; c1ccc(-c2cc(-c3ccc4c(c3)C3(c5ccccc5-c5ccccc53)c3ccccc3-4)nc(-c3cccc(-c4nc5ccccn5c4-c4ccccc4)c3)n2)cc1]

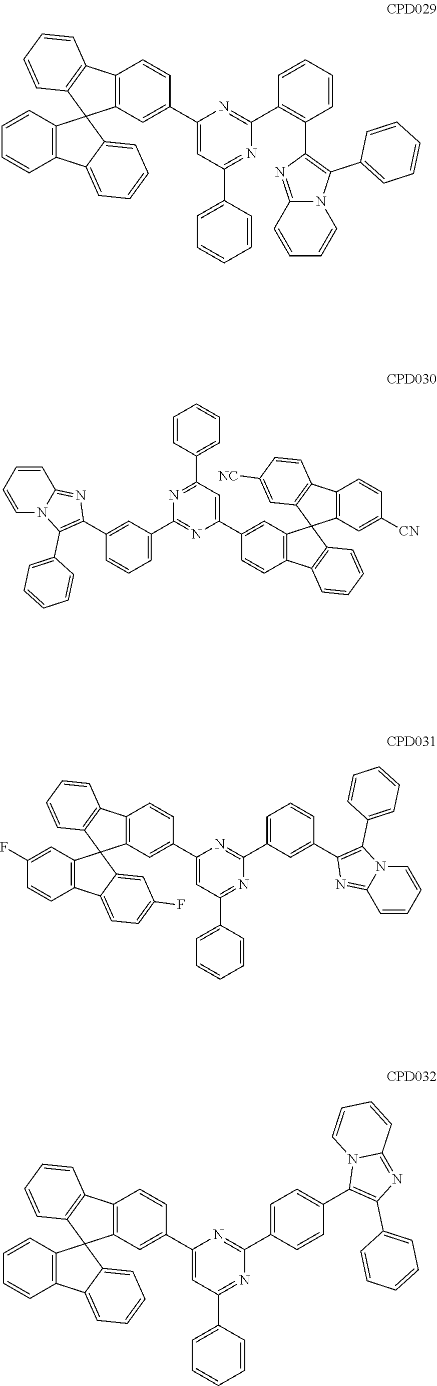 [SMILES: c1ccc(-c2cc(-c3ccc4c(c3)C3(c5ccccc5-c5ccccc53)c3ccccc3-4)nc(-c3ccccc3-c3nc4ccccn4c3-c3ccccc3)n2)cc1 ; N#Cc1ccc2c(c1)C1(c3ccccc3-c3ccc(-c4cc(-c5ccccc5)nc(-c5cccc(-c6nc7ccccn7c6-c6ccccc6)c5)n4)cc31)c1cc(C#N)ccc1-2 ; Fc1ccc2c(c1)C1(c3ccccc3-c3ccc(-c4cc(-c5ccccc5)nc(-c5cccc(-c6nc7ccccn7c6-c6ccccc6)c5)n4)cc31)c1cc(F)ccc1-2 ; c1ccc(-c2cc(-c3ccc4c(c3)C3(c5ccccc5-c5ccccc53)c3ccccc3-4)nc(-c3ccc(-c4c(-c5ccccc5)nc5ccccn45)cc3)n2)cc1]

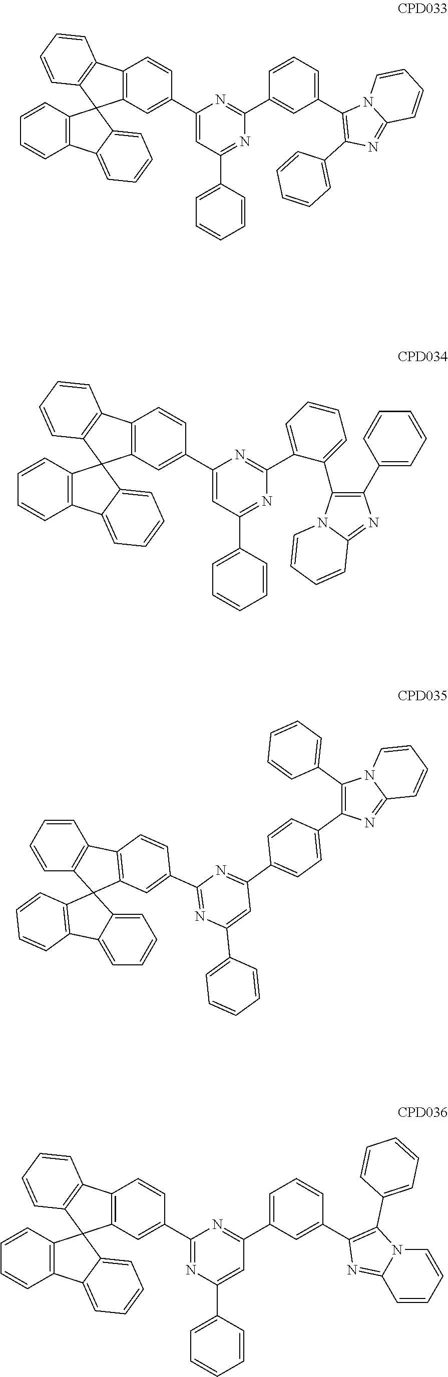 [SMILES: c1ccc(-c2cc(-c3ccc4c(c3)C3(c5ccccc5-c5ccccc53)c3ccccc3-4)nc(-c3cccc(-c4c(-c5ccccc5)nc5ccccn45)c3)n2)cc1 ; c1ccc(-c2cc(-c3ccc4c(c3)C3(c5ccccc5-c5ccccc53)c3ccccc3-4)nc(-c3ccccc3-c3c(-c4ccccc4)nc4ccccn34)n2)cc1 ; c1ccc(-c2cc(-c3ccc(-c4nc5ccccn5c4-c4ccccc4)cc3)nc(-c3ccc4c(c3)C3(c5ccccc5-c5ccccc53)c3ccccc3-4)n2)cc1 ; c1ccc(-c2cc(-c3cccc(-c4nc5ccccn5c4-c4ccccc4)c3)nc(-c3ccc4c(c3)C3(c5ccccc5-c5ccccc53)c3ccccc3-4)n2)cc1]

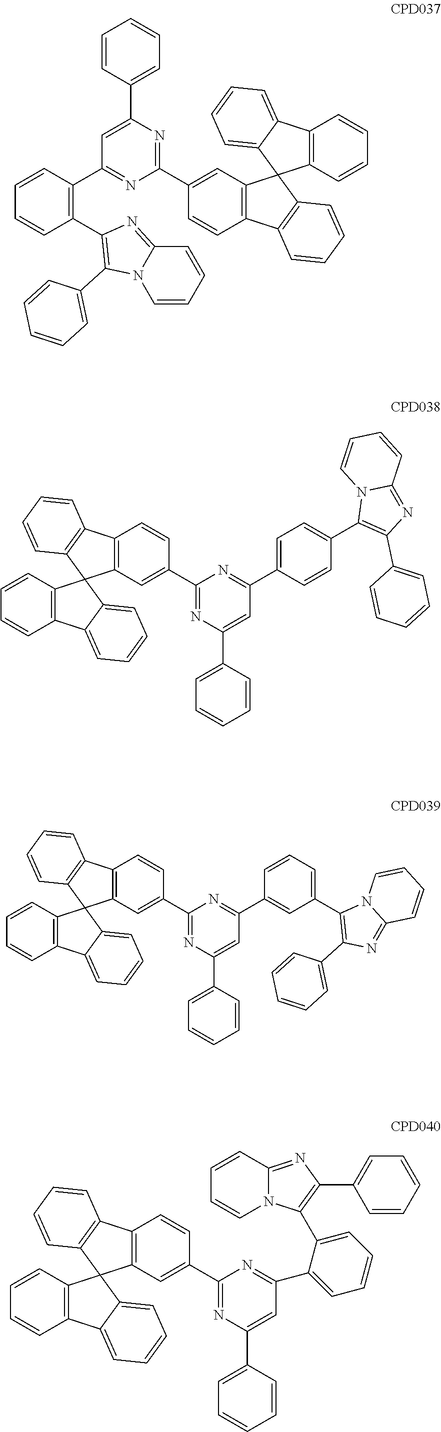 [SMILES: c1ccc(-c2cc(-c3ccccc3-c3nc4ccccn4c3-c3ccccc3)nc(-c3ccc4c(c3)C3(c5ccccc5-c5ccccc53)c3ccccc3-4)n2)cc1 ; c1ccc(-c2cc(-c3ccc(-c4c(-c5ccccc5)nc5ccccn45)cc3)nc(-c3ccc4c(c3)C3(c5ccccc5-c5ccccc53)c3ccccc3-4)n2)cc1 ; c1ccc(-c2cc(-c3cccc(-c4c(-c5ccccc5)nc5ccccn45)c3)nc(-c3ccc4c(c3)C3(c5ccccc5-c5ccccc53)c3ccccc3-4)n2)cc1 ; c1ccc(-c2cc(-c3ccccc3-c3c(-c4ccccc4)nc4ccccn34)nc(-c3ccc4c(c3)C3(c5ccccc5-c5ccccc53)c3ccccc3-4)n2)cc1]

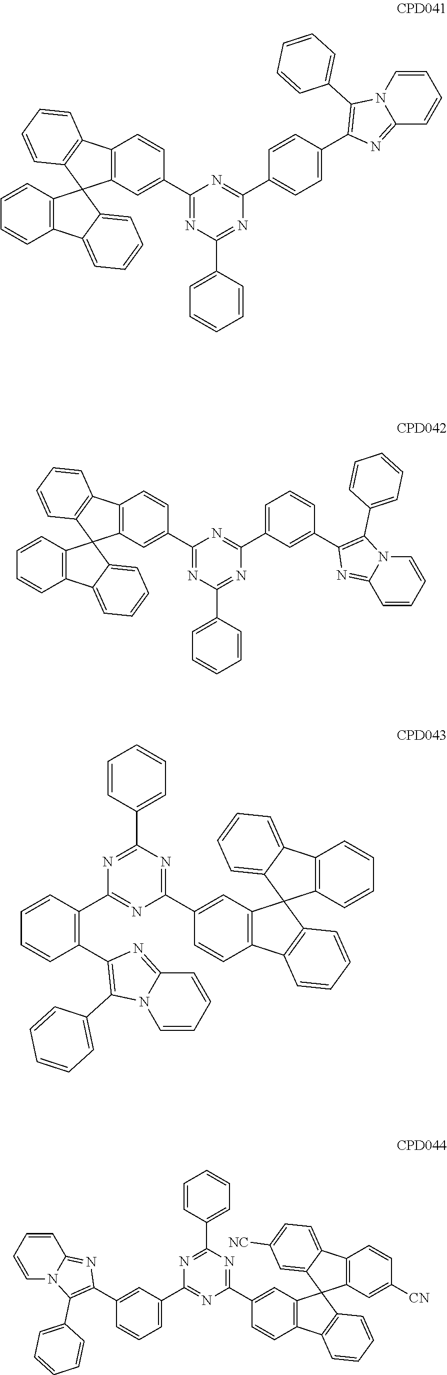 [SMILES: c1ccc(-c2nc(-c3ccc(-c4nc5ccccn5c4-c4ccccc4)cc3)nc(-c3ccc4c(c3)C3(c5ccccc5-c5ccccc53)c3ccccc3-4)n2)cc1 ; c1ccc(-c2nc(-c3cccc(-c4nc5ccccn5c4-c4ccccc4)c3)nc(-c3ccc4c(c3)C3(c5ccccc5-c5ccccc53)c3ccccc3-4)n2)cc1 ; c1ccc(-c2nc(-c3ccc4c(c3)C3(c5ccccc5-c5ccccc53)c3ccccc3-4)nc(-c3ccccc3-c3nc4ccccn4c3-c3ccccc3)n2)cc1 ; N#Cc1ccc2c(c1)C1(c3ccccc3-c3ccc(-c4nc(-c5ccccc5)nc(-c5cccc(-c6nc7ccccn7c6-c6ccccc6)c5)n4)cc31)c1cc(C#N)ccc1-2]

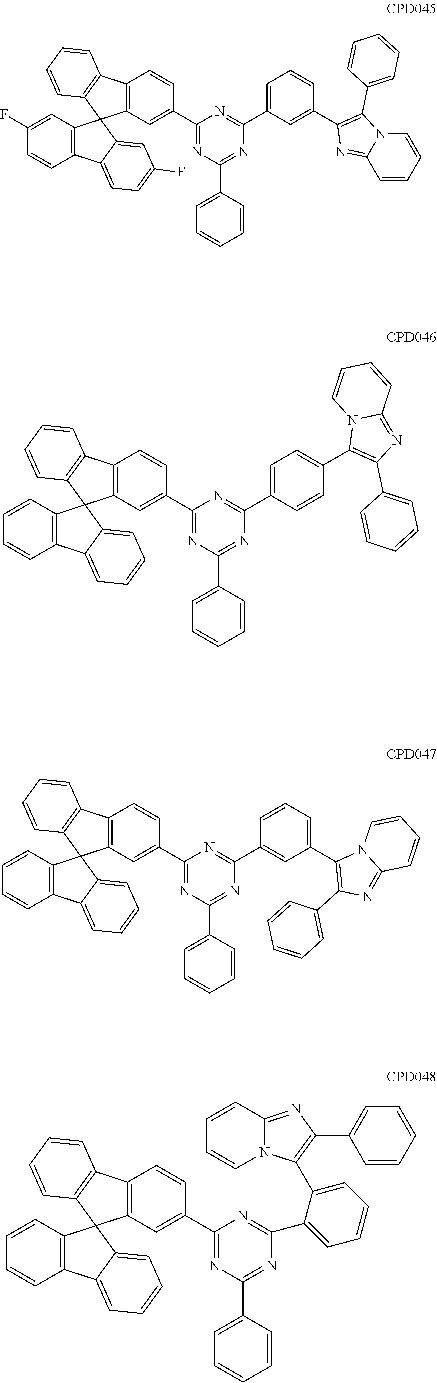 [SMILES: Fc1ccc2c(c1)C1(c3ccccc3-c3ccc(-c4nc(-c5ccccc5)nc(-c5cccc(-c6nc7ccccn7c6-c6ccccc6)c5)n4)cc31)c1cc(F)ccc1-2 ; c1ccc(-c2nc(-c3ccc(-c4c(-c5ccccc5)nc5ccccn45)cc3)nc(-c3ccc4c(c3)C3(c5ccccc5-c5ccccc53)c3ccccc3-4)n2)cc1 ; c1ccc(-c2nc(-c3cccc(-c4c(-c5ccccc5)nc5ccccn45)c3)nc(-c3ccc4c(c3)C3(c5ccccc5-c5ccccc53)c3ccccc3-4)n2)cc1 ; c1ccc(-c2nc(-c3ccc4c(c3)C3(c5ccccc5-c5ccccc53)c3ccccc3-4)nc(-c3ccccc3-c3c(-c4ccccc4)nc4ccccn34)n2)cc1]

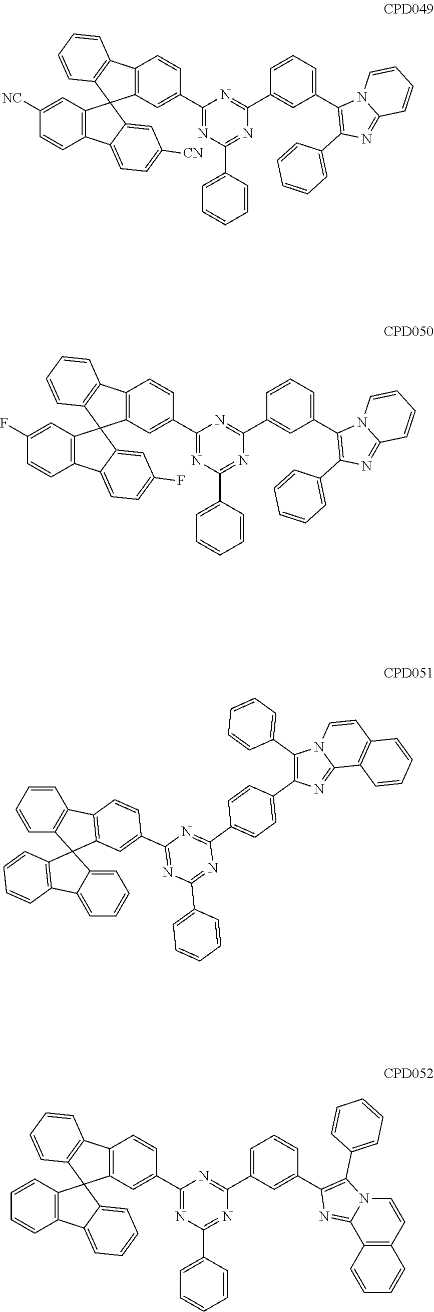 [SMILES: N#Cc1ccc2c(c1)C1(c3ccccc3-c3ccc(-c4nc(-c5ccccc5)nc(-c5cccc(-c6c(-c7ccccc7)nc7ccccn67)c5)n4)cc31)c1cc(C#N)ccc1-2 ; Fc1ccc2c(c1)C1(c3ccccc3-c3ccc(-c4nc(-c5ccccc5)nc(-c5cccc(-c6c(-c7ccccc7)nc7ccccn67)c5)n4)cc31)c1cc(F)ccc1-2 ; c1ccc(-c2nc(-c3ccc(-c4nc5c6ccccc6ccn5c4-c4ccccc4)cc3)nc(-c3ccc4c(c3)C3(c5ccccc5-c5ccccc53)c3ccccc3-4)n2)cc1 ; c1ccc(-c2nc(-c3cccc(-c4nc5c6ccccc6ccn5c4-c4ccccc4)c3)nc(-c3ccc4c(c3)C3(c5ccccc5-c5ccccc53)c3ccccc3-4)n2)cc1]

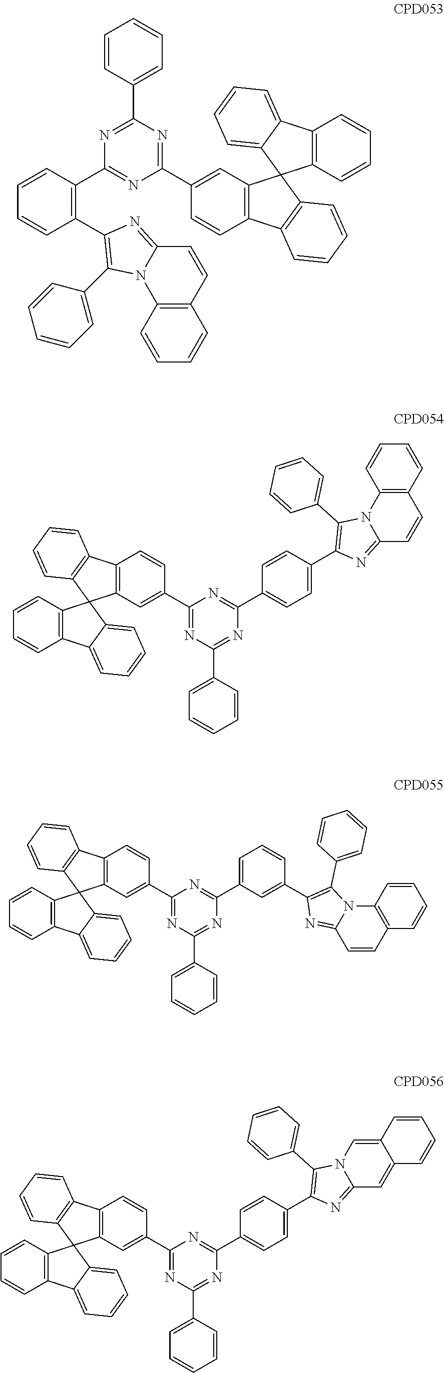 [SMILES: c1ccc(-c2nc(-c3ccc4c(c3)C3(c5ccccc5-c5ccccc53)c3ccccc3-4)nc(-c3ccccc3-c3nc4ccc5ccccc5n4c3-c3ccccc3)n2)cc1 ; c1ccc(-c2nc(-c3ccc(-c4nc5ccc6ccccc6n5c4-c4ccccc4)cc3)nc(-c3ccc4c(c3)C3(c5ccccc5-c5ccccc53)c3ccccc3-4)n2)cc1 ; c1ccc(-c2nc(-c3cccc(-c4nc5ccc6ccccc6n5c4-c4ccccc4)c3)nc(-c3ccc4c(c3)C3(c5ccccc5-c5ccccc53)c3ccccc3-4)n2)cc1 ; c1ccc(-c2nc(-c3ccc(-c4nc5cc6ccccc6cn5c4-c4ccccc4)cc3)nc(-c3ccc4c(c3)C3(c5ccccc5-c5ccccc53)c3ccccc3-4)n2)cc1]

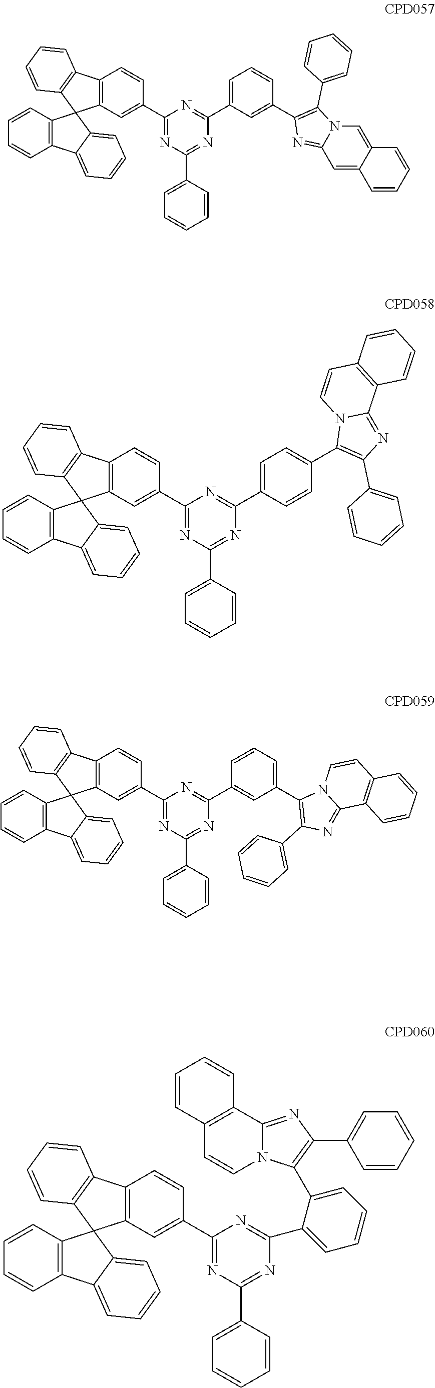 [SMILES: c1ccc(-c2nc(-c3cccc(-c4nc5cc6ccccc6cn5c4-c4ccccc4)c3)nc(-c3ccc4c(c3)C3(c5ccccc5-c5ccccc53)c3ccccc3-4)n2)cc1 ; c1ccc(-c2nc(-c3ccc(-c4c(-c5ccccc5)nc5c6ccccc6ccn45)cc3)nc(-c3ccc4c(c3)C3(c5ccccc5-c5ccccc53)c3ccccc3-4)n2)cc1 ; c1ccc(-c2nc(-c3cccc(-c4c(-c5ccccc5)nc5c6ccccc6ccn45)c3)nc(-c3ccc4c(c3)C3(c5ccccc5-c5ccccc53)c3ccccc3-4)n2)cc1 ; c1ccc(-c2nc(-c3ccc4c(c3)C3(c5ccccc5-c5ccccc53)c3ccccc3-4)nc(-c3ccccc3-c3c(-c4ccccc4)nc4c5ccccc5ccn34)n2)cc1]

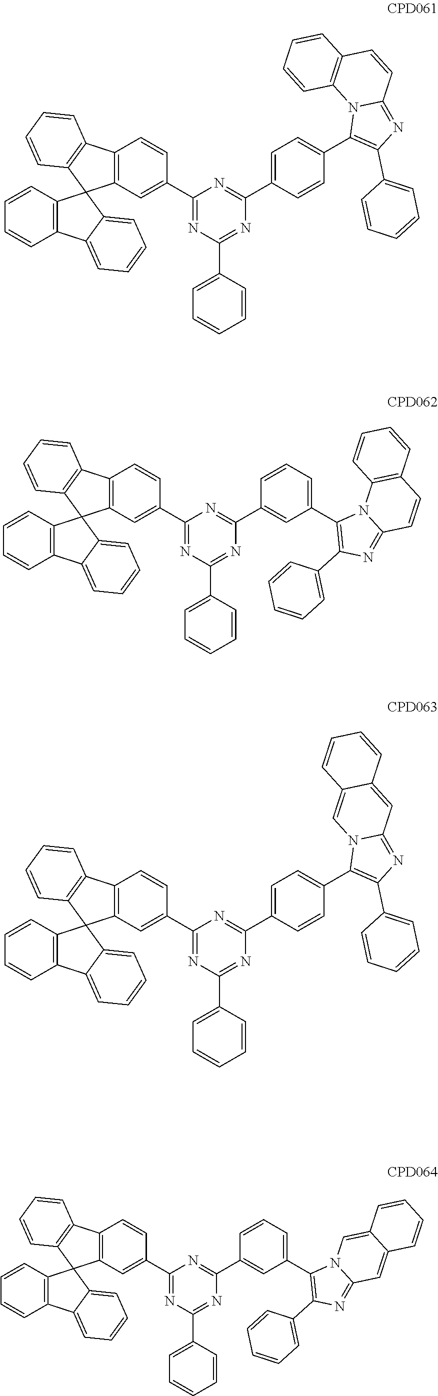 [SMILES: c1ccc(-c2nc(-c3ccc(-c4c(-c5ccccc5)nc5ccc6ccccc6n45)cc3)nc(-c3ccc4c(c3)C3(c5ccccc5-c5ccccc53)c3ccccc3-4)n2)cc1 ; c1ccc(-c2nc(-c3cccc(-c4c(-c5ccccc5)nc5ccc6ccccc6n45)c3)nc(-c3ccc4c(c3)C3(c5ccccc5-c5ccccc53)c3ccccc3-4)n2)cc1 ; c1ccc(-c2nc(-c3ccc(-c4c(-c5ccccc5)nc5cc6ccccc6cn45)cc3)nc(-c3ccc4c(c3)C3(c5ccccc5-c5ccccc53)c3ccccc3-4)n2)cc1 ; c1ccc(-c2nc(-c3cccc(-c4c(-c5ccccc5)nc5cc6ccccc6cn45)c3)nc(-c3ccc4c(c3)C3(c5ccccc5-c5ccccc53)c3ccccc3-4)n2)cc1]

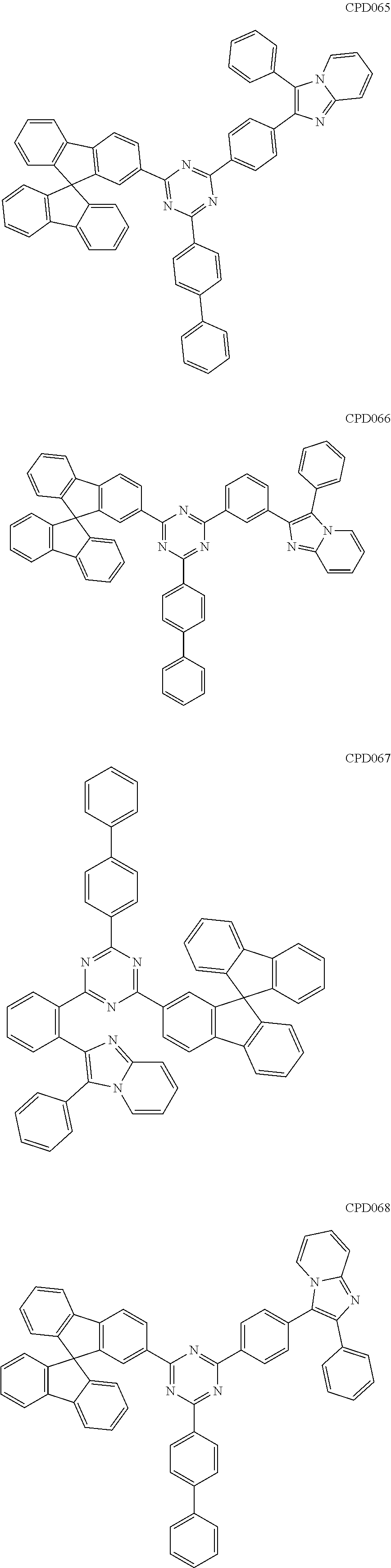 [SMILES: c1ccc(-c2ccc(-c3nc(-c4ccc(-c5nc6ccccn6c5-c5ccccc5)cc4)nc(-c4ccc5c(c4)C4(c6ccccc6-c6ccccc64)c4ccccc4-5)n3)cc2)cc1 ; c1ccc(-c2ccc(-c3nc(-c4cccc(-c5nc6ccccn6c5-c5ccccc5)c4)nc(-c4ccc5c(c4)C4(c6ccccc6-c6ccccc64)c4ccccc4-5)n3)cc2)cc1 ; c1ccc(-c2ccc(-c3nc(-c4ccc5c(c4)C4(c6ccccc6-c6ccccc64)c4ccccc4-5)nc(-c4ccccc4-c4nc5ccccn5c4-c4ccccc4)n3)cc2)cc1 ; c1ccc(-c2ccc(-c3nc(-c4ccc(-c5c(-c6ccccc6)nc6ccccn56)cc4)nc(-c4ccc5c(c4)C4(c6ccccc6-c6ccccc64)c4ccccc4-5)n3)cc2)cc1]

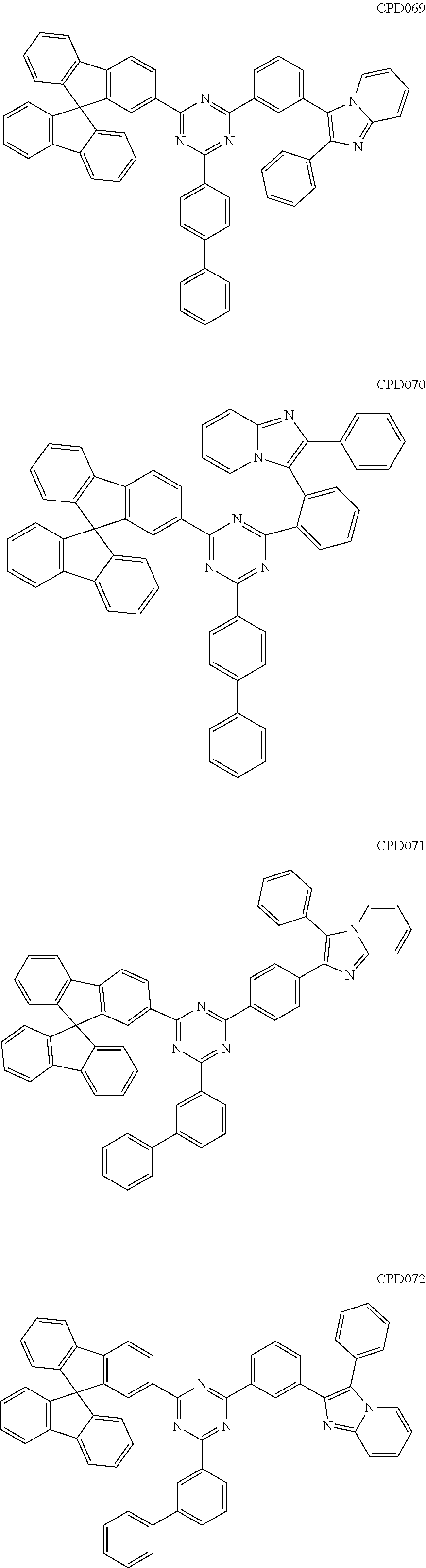 [SMILES: c1ccc(-c2ccc(-c3nc(-c4cccc(-c5c(-c6ccccc6)nc6ccccn56)c4)nc(-c4ccc5c(c4)C4(c6ccccc6-c6ccccc64)c4ccccc4-5)n3)cc2)cc1 ; c1ccc(-c2ccc(-c3nc(-c4ccc5c(c4)C4(c6ccccc6-c6ccccc64)c4ccccc4-5)nc(-c4ccccc4-c4c(-c5ccccc5)nc5ccccn45)n3)cc2)cc1 ; c1ccc(-c2cccc(-c3nc(-c4ccc(-c5nc6ccccn6c5-c5ccccc5)cc4)nc(-c4ccc5c(c4)C4(c6ccccc6-c6ccccc64)c4ccccc4-5)n3)c2)cc1 ; c1ccc(-c2cccc(-c3nc(-c4cccc(-c5nc6ccccn6c5-c5ccccc5)c4)nc(-c4ccc5c(c4)C4(c6ccccc6-c6ccccc64)c4ccccc4-5)n3)c2)cc1]

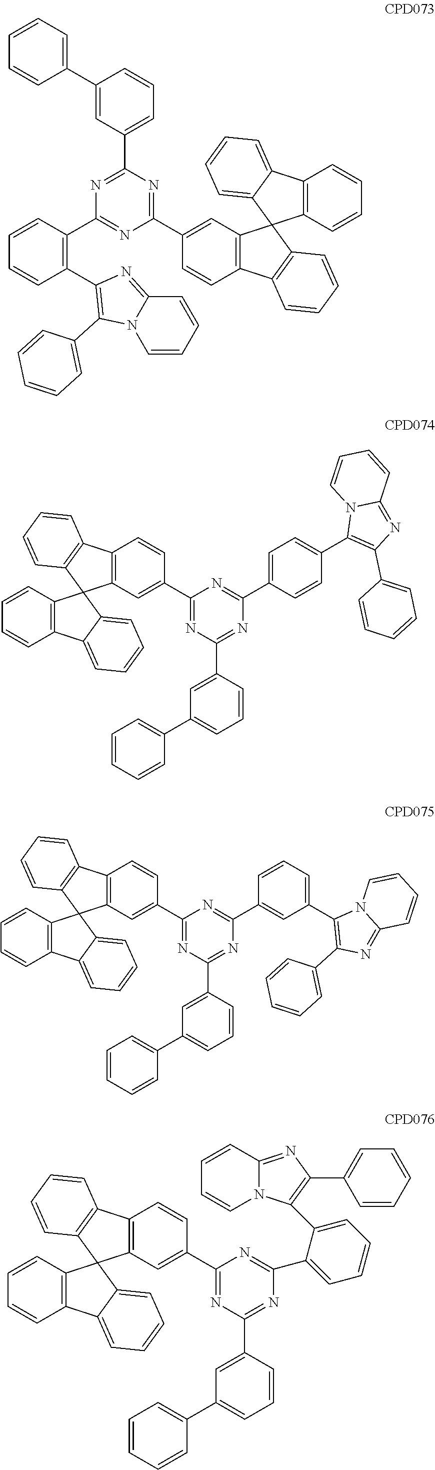 [SMILES: c1ccc(-c2cccc(-c3nc(-c4ccc5c(c4)C4(c6ccccc6-c6ccccc64)c4ccccc4-5)nc(-c4ccccc4-c4nc5ccccn5c4-c4ccccc4)n3)c2)cc1 ; c1ccc(-c2cccc(-c3nc(-c4ccc(-c5c(-c6ccccc6)nc6ccccn56)cc4)nc(-c4ccc5c(c4)C4(c6ccccc6-c6ccccc64)c4ccccc4-5)n3)c2)cc1 ; c1ccc(-c2cccc(-c3nc(-c4cccc(-c5c(-c6ccccc6)nc6ccccn56)c4)nc(-c4ccc5c(c4)C4(c6ccccc6-c6ccccc64)c4ccccc4-5)n3)c2)cc1 ; c1ccc(-c2cccc(-c3nc(-c4ccc5c(c4)C4(c6ccccc6-c6ccccc64)c4ccccc4-5)nc(-c4ccccc4-c4c(-c5ccccc5)nc5ccccn45)n3)c2)cc1]

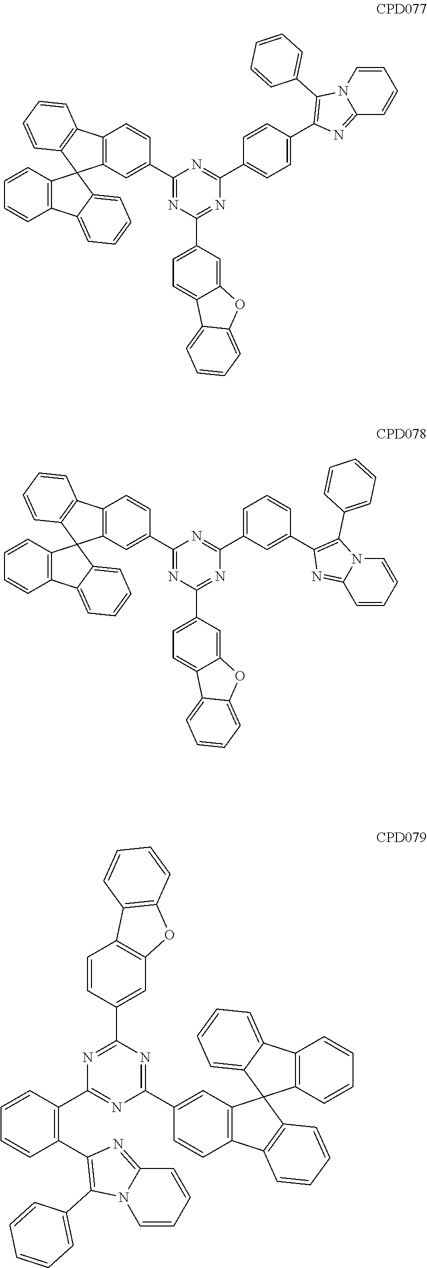 [SMILES: c1ccc(-c2c(-c3ccc(-c4nc(-c5ccc6c(c5)C5(c7ccccc7-c7ccccc75)c5ccccc5-6)nc(-c5ccc6c(c5)oc5ccccc56)n4)cc3)nc3ccccn23)cc1 ; c1ccc(-c2c(-c3cccc(-c4nc(-c5ccc6c(c5)C5(c7ccccc7-c7ccccc75)c5ccccc5-6)nc(-c5ccc6c(c5)oc5ccccc56)n4)c3)nc3ccccn23)cc1 ; c1ccc(-c2c(-c3ccccc3-c3nc(-c4ccc5c(c4)C4(c6ccccc6-c6ccccc64)c4ccccc4-5)nc(-c4ccc5c(c4)oc4ccccc45)n3)nc3ccccn23)cc1]

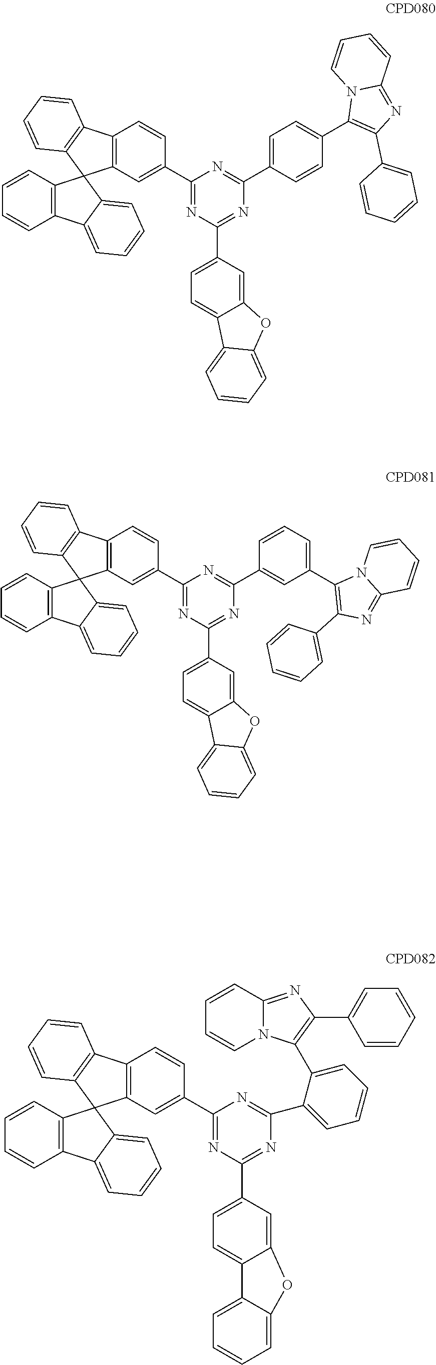 [SMILES: c1ccc(-c2nc3ccccn3c2-c2ccc(-c3nc(-c4ccc5c(c4)C4(c6ccccc6-c6ccccc64)c4ccccc4-5)nc(-c4ccc5c(c4)oc4ccccc45)n3)cc2)cc1 ; c1ccc(-c2nc3ccccn3c2-c2cccc(-c3nc(-c4ccc5c(c4)C4(c6ccccc6-c6ccccc64)c4ccccc4-5)nc(-c4ccc5c(c4)oc4ccccc45)n3)c2)cc1 ; c1ccc(-c2nc3ccccn3c2-c2ccccc2-c2nc(-c3ccc4c(c3)C3(c5ccccc5-c5ccccc53)c3ccccc3-4)nc(-c3ccc4c(c3)oc3ccccc34)n2)cc1]

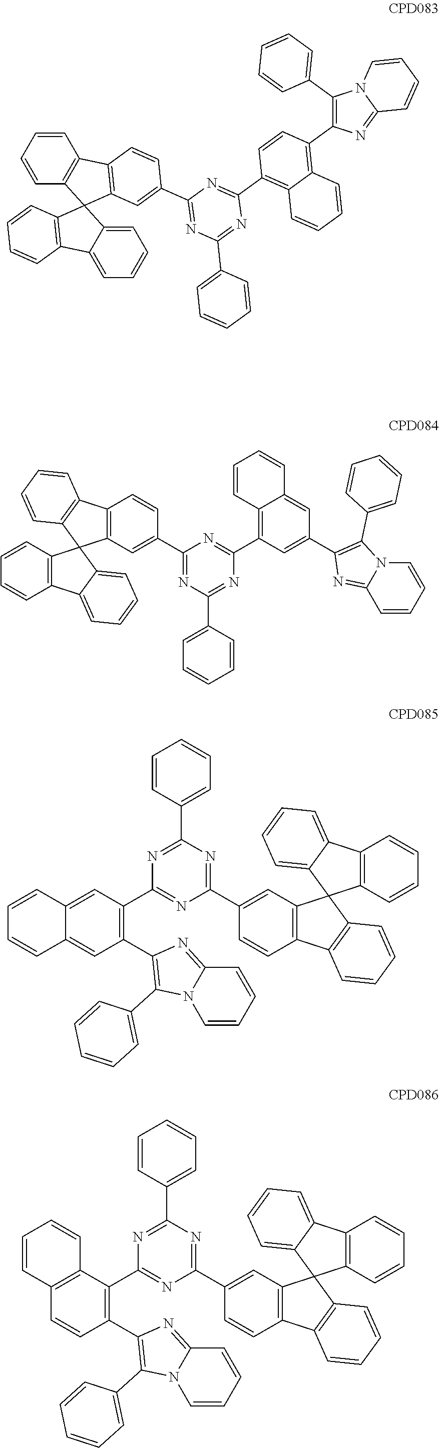 [SMILES: c1ccc(-c2nc(-c3ccc4c(c3)C3(c5ccccc5-c5ccccc53)c3ccccc3-4)nc(-c3ccc(-c4nc5ccccn5c4-c4ccccc4)c4ccccc34)n2)cc1 ; c1ccc(-c2nc(-c3ccc4c(c3)C3(c5ccccc5-c5ccccc53)c3ccccc3-4)nc(-c3cc(-c4nc5ccccn5c4-c4ccccc4)cc4ccccc34)n2)cc1 ; c1ccc(-c2nc(-c3ccc4c(c3)C3(c5ccccc5-c5ccccc53)c3ccccc3-4)nc(-c3cc4ccccc4cc3-c3nc4ccccn4c3-c3ccccc3)n2)cc1 ; c1ccc(-c2nc(-c3ccc4c(c3)C3(c5ccccc5-c5ccccc53)c3ccccc3-4)nc(-c3c(-c4nc5ccccn5c4-c4ccccc4)ccc4ccccc34)n2)cc1]

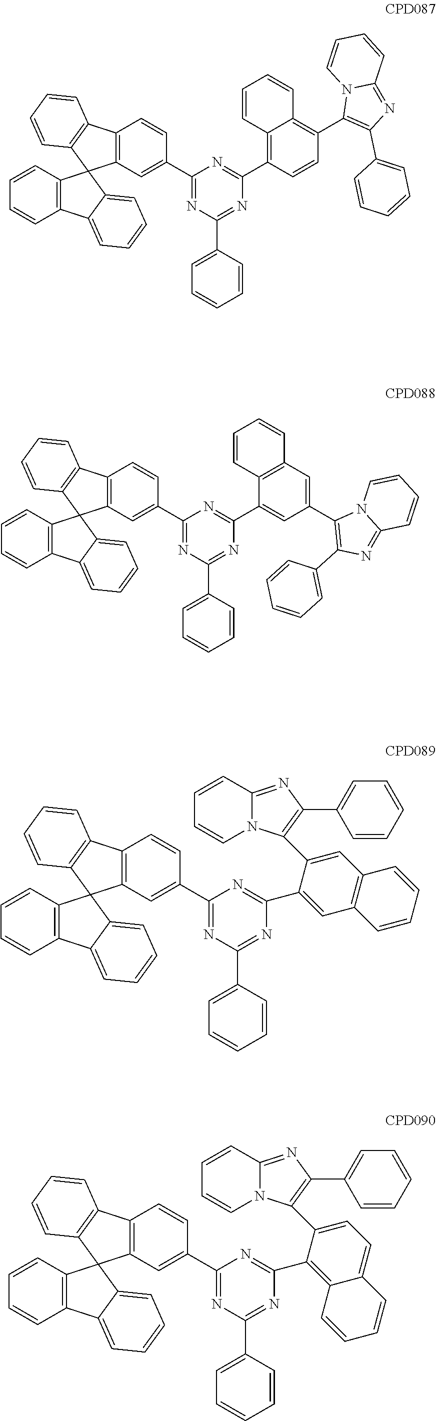 [SMILES: c1ccc(-c2nc(-c3ccc4c(c3)C3(c5ccccc5-c5ccccc53)c3ccccc3-4)nc(-c3ccc(-c4c(-c5ccccc5)nc5ccccn45)c4ccccc34)n2)cc1 ; c1ccc(-c2nc(-c3ccc4c(c3)C3(c5ccccc5-c5ccccc53)c3ccccc3-4)nc(-c3cc(-c4c(-c5ccccc5)nc5ccccn45)cc4ccccc34)n2)cc1 ; c1ccc(-c2nc(-c3ccc4c(c3)C3(c5ccccc5-c5ccccc53)c3ccccc3-4)nc(-c3cc4ccccc4cc3-c3c(-c4ccccc4)nc4ccccn34)n2)cc1 ; c1ccc(-c2nc(-c3ccc4c(c3)C3(c5ccccc5-c5ccccc53)c3ccccc3-4)nc(-c3c(-c4c(-c5ccccc5)nc5ccccn45)ccc4ccccc34)n2)cc1]

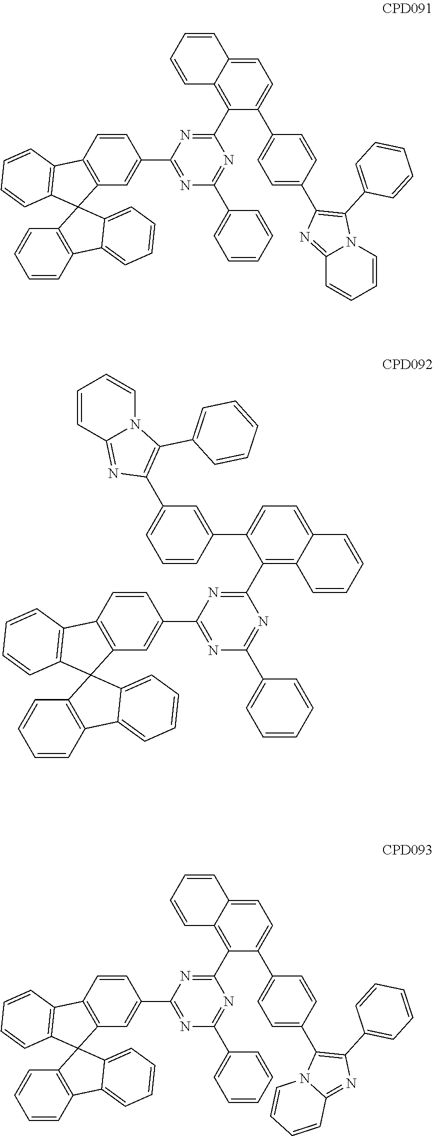 [SMILES: c1ccc(-c2nc(-c3ccc4c(c3)C3(c5ccccc5-c5ccccc53)c3ccccc3-4)nc(-c3c(-c4ccc(-c5nc6ccccn6c5-c5ccccc5)cc4)ccc4ccccc34)n2)cc1 ; c1ccc(-c2nc(-c3ccc4c(c3)C3(c5ccccc5-c5ccccc53)c3ccccc3-4)nc(-c3c(-c4cccc(-c5nc6ccccn6c5-c5ccccc5)c4)ccc4ccccc34)n2)cc1 ; c1ccc(-c2nc(-c3ccc4c(c3)C3(c5ccccc5-c5ccccc53)c3ccccc3-4)nc(-c3c(-c4ccc(-c5c(-c6ccccc6)nc6ccccn56)cc4)ccc4ccccc34)n2)cc1]

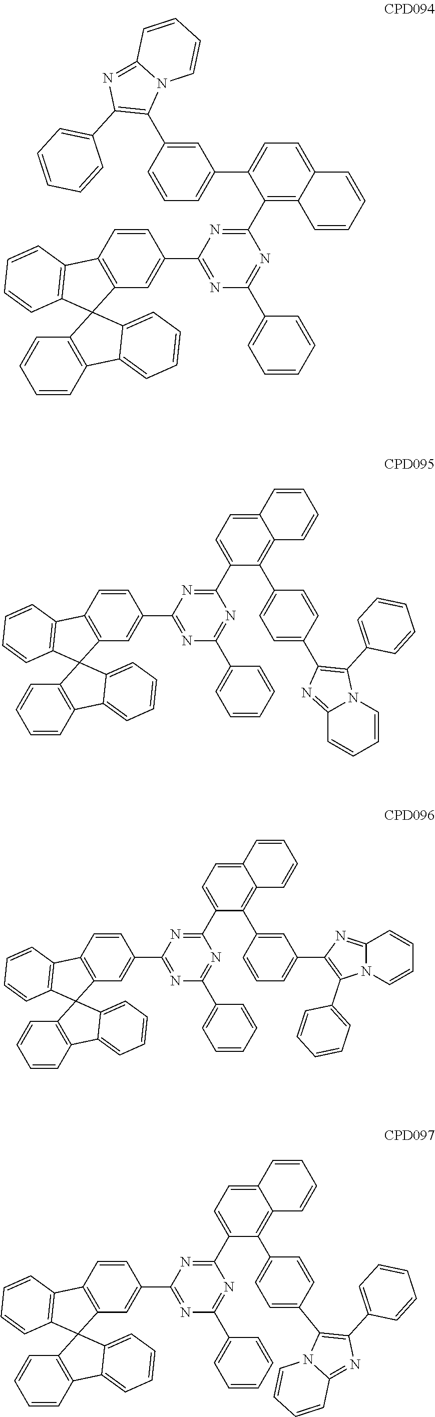 [SMILES: c1ccc(-c2nc(-c3ccc4c(c3)C3(c5ccccc5-c5ccccc53)c3ccccc3-4)nc(-c3c(-c4cccc(-c5c(-c6ccccc6)nc6ccccn56)c4)ccc4ccccc34)n2)cc1 ; c1ccc(-c2nc(-c3ccc4c(c3)C3(c5ccccc5-c5ccccc53)c3ccccc3-4)nc(-c3ccc4ccccc4c3-c3ccc(-c4nc5ccccn5c4-c4ccccc4)cc3)n2)cc1 ; c1ccc(-c2nc(-c3ccc4c(c3)C3(c5ccccc5-c5ccccc53)c3ccccc3-4)nc(-c3ccc4ccccc4c3-c3cccc(-c4nc5ccccn5c4-c4ccccc4)c3)n2)cc1 ; c1ccc(-c2nc(-c3ccc4c(c3)C3(c5ccccc5-c5ccccc53)c3ccccc3-4)nc(-c3ccc4ccccc4c3-c3ccc(-c4c(-c5ccccc5)nc5ccccn45)cc3)n2)cc1]

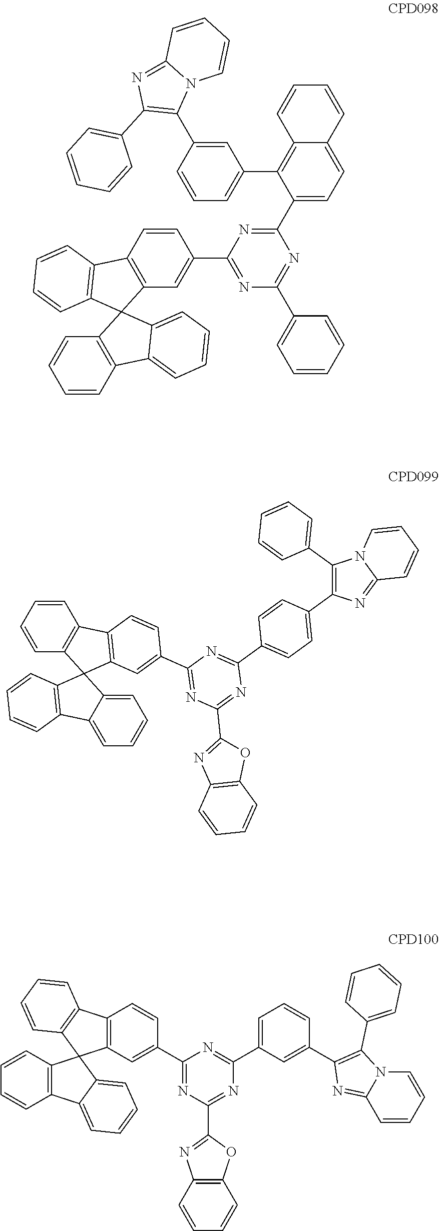 [SMILES: c1ccc(-c2nc(-c3ccc4c(c3)C3(c5ccccc5-c5ccccc53)c3ccccc3-4)nc(-c3ccc4ccccc4c3-c3cccc(-c4c(-c5ccccc5)nc5ccccn45)c3)n2)cc1 ; c1ccc(-c2c(-c3ccc(-c4nc(-c5ccc6c(c5)C5(c7ccccc7-c7ccccc75)c5ccccc5-6)nc(-c5nc6ccccc6o5)n4)cc3)nc3ccccn23)cc1 ; c1ccc(-c2c(-c3cccc(-c4nc(-c5ccc6c(c5)C5(c7ccccc7-c7ccccc75)c5ccccc5-6)nc(-c5nc6ccccc6o5)n4)c3)nc3ccccn23)cc1]

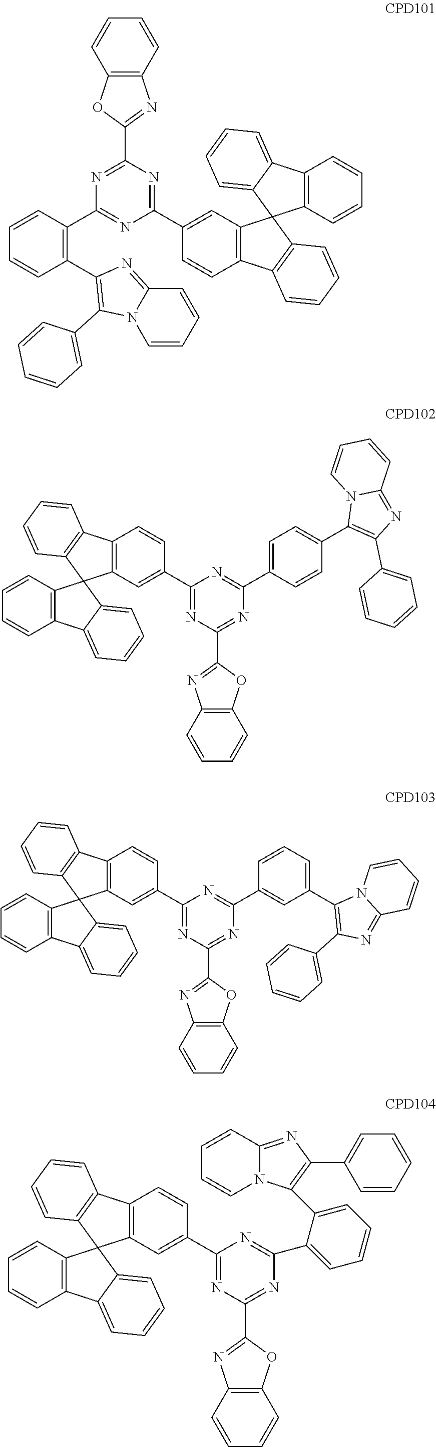 [SMILES: c1ccc(-c2c(-c3ccccc3-c3nc(-c4ccc5c(c4)C4(c6ccccc6-c6ccccc64)c4ccccc4-5)nc(-c4nc5ccccc5o4)n3)nc3ccccn23)cc1 ; c1ccc(-c2nc3ccccn3c2-c2ccc(-c3nc(-c4ccc5c(c4)C4(c6ccccc6-c6ccccc64)c4ccccc4-5)nc(-c4nc5ccccc5o4)n3)cc2)cc1 ; c1ccc(-c2nc3ccccn3c2-c2cccc(-c3nc(-c4ccc5c(c4)C4(c6ccccc6-c6ccccc64)c4ccccc4-5)nc(-c4nc5ccccc5o4)n3)c2)cc1 ; c1ccc(-c2nc3ccccn3c2-c2ccccc2-c2nc(-c3ccc4c(c3)C3(c5ccccc5-c5ccccc53)c3ccccc3-4)nc(-c3nc4ccccc4o3)n2)cc1]

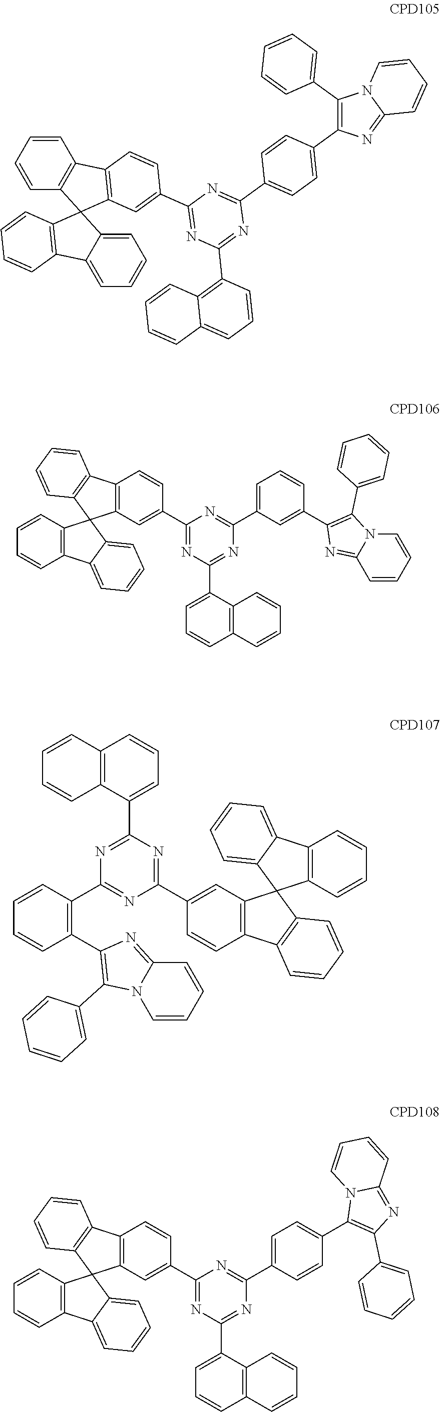 [SMILES: c1ccc(-c2c(-c3ccc(-c4nc(-c5ccc6c(c5)C5(c7ccccc7-c7ccccc75)c5ccccc5-6)nc(-c5cccc6ccccc56)n4)cc3)nc3ccccn23)cc1 ; c1ccc(-c2c(-c3cccc(-c4nc(-c5ccc6c(c5)C5(c7ccccc7-c7ccccc75)c5ccccc5-6)nc(-c5cccc6ccccc56)n4)c3)nc3ccccn23)cc1 ; c1ccc(-c2c(-c3ccccc3-c3nc(-c4ccc5c(c4)C4(c6ccccc6-c6ccccc64)c4ccccc4-5)nc(-c4cccc5ccccc45)n3)nc3ccccn23)cc1 ; c1ccc(-c2nc3ccccn3c2-c2ccc(-c3nc(-c4ccc5c(c4)C4(c6ccccc6-c6ccccc64)c4ccccc4-5)nc(-c4cccc5ccccc45)n3)cc2)cc1]

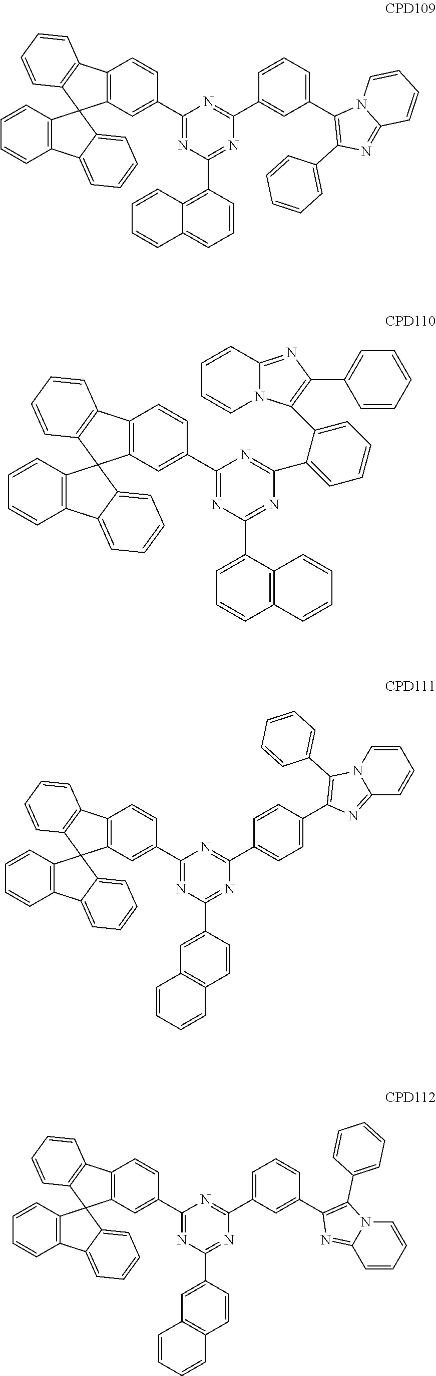 [SMILES: c1ccc(-c2nc3ccccn3c2-c2cccc(-c3nc(-c4ccc5c(c4)C4(c6ccccc6-c6ccccc64)c4ccccc4-5)nc(-c4cccc5ccccc45)n3)c2)cc1 ; c1ccc(-c2nc3ccccn3c2-c2ccccc2-c2nc(-c3ccc4c(c3)C3(c5ccccc5-c5ccccc53)c3ccccc3-4)nc(-c3cccc4ccccc34)n2)cc1 ; c1ccc(-c2c(-c3ccc(-c4nc(-c5ccc6c(c5)C5(c7ccccc7-c7ccccc75)c5ccccc5-6)nc(-c5ccc6ccccc6c5)n4)cc3)nc3ccccn23)cc1 ; c1ccc(-c2c(-c3cccc(-c4nc(-c5ccc6c(c5)C5(c7ccccc7-c7ccccc75)c5ccccc5-6)nc(-c5ccc6ccccc6c5)n4)c3)nc3ccccn23)cc1]

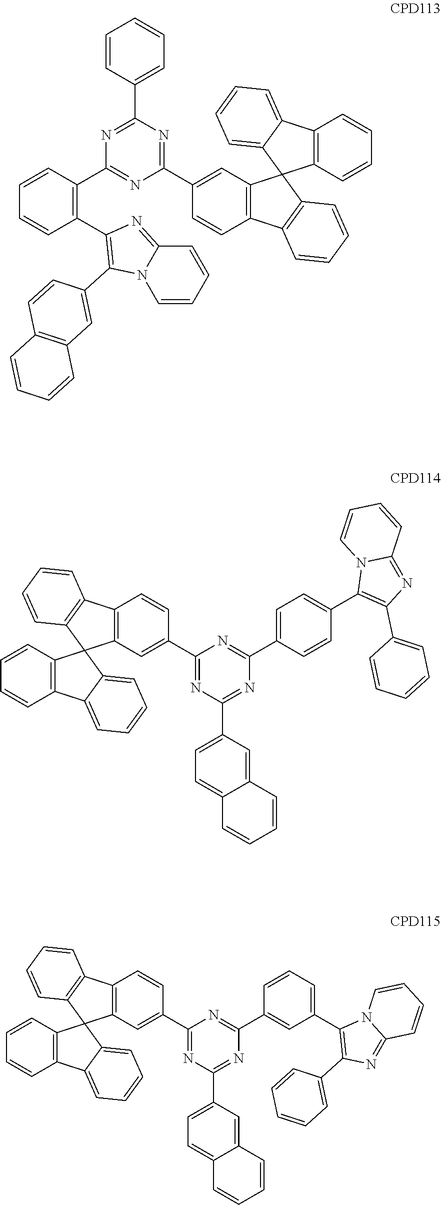 [SMILES: c1ccc(-c2nc(-c3ccc4c(c3)C3(c5ccccc5-c5ccccc53)c3ccccc3-4)nc(-c3ccccc3-c3nc4ccccn4c3-c3ccc4ccccc4c3)n2)cc1 ; c1ccc(-c2nc3ccccn3c2-c2ccc(-c3nc(-c4ccc5c(c4)C4(c6ccccc6-c6ccccc64)c4ccccc4-5)nc(-c4ccc5ccccc5c4)n3)cc2)cc1 ; c1ccc(-c2nc3ccccn3c2-c2cccc(-c3nc(-c4ccc5c(c4)C4(c6ccccc6-c6ccccc64)c4ccccc4-5)nc(-c4ccc5ccccc5c4)n3)c2)cc1]

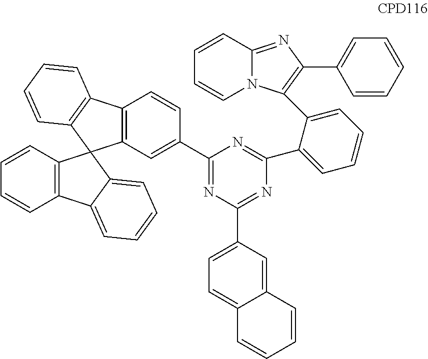 [SMILES: c1ccc(-c2nc3ccccn3c2-c2ccccc2-c2nc(-c3ccc4c(c3)C3(c5ccccc5-c5ccccc53)c3ccccc3-4)nc(-c3ccc4ccccc4c3)n2)cc1]

One of the purposes of the present invention is to provide application of the spiro compound in an organic electroluminescent device.

One of the purposes of the present invention is to provide use of the spiro compound as an electron injection layer or an electron transport layer of an organic electroluminescent device.

By connecting N heterocyclic ring with imidazopyridine ring on spirofluorene, the material of the present invention has the advantages of high optical and electrical stability, low sublimation temperature, low driving voltage, high luminous efficiency, long device service life and the like, and can be used in an organic electroluminescent device. In particular, the compound has the possibility of being applied to the AMOLED industry as an electron injection or transport material.

### BRIEF DESCRIPTION OF DRAWINGS

FIG. 1 is a diagram showing the ¹HNMR spectrum of a compound CPD075.

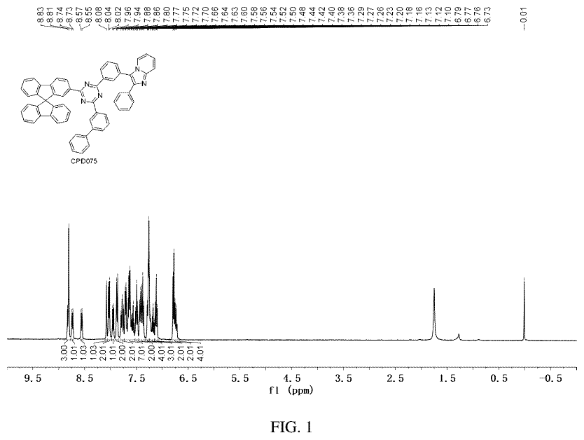

### DETAILED DESCRIPTION OF EMBODIMENTS

A compound, namely a spiro compound, of the present invention has a structure as shown in a formula (1),

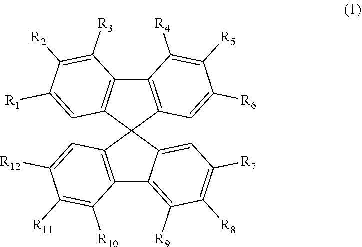

where R₁-R₁₂ are independently selected from hydrogen, deuterium, halogen, cyano, hydroxyl, sulfhydryl, amino, substituted or unsubstituted C₁-C₁₀ alkyl, substituted or unsubstituted C₁-C₁₀heteroalkyl, substituted or unsubstituted C₃-C₂₀ cycloalkyl, substituted or unsubstituted C₃-C₂₀heterocycloalkyl, substituted or unsubstituted C₂-C₁₀ alkenyl, substituted or unsubstituted C₂-C₁₀ alkynyl, substituted or unsubstituted C₆-C₃₀ aryl, substituted or unsubstituted C₂-C₃₀ heteroaryl, substituted or unsubstituted tri-C₁-C₁₀alkylsilyl, substituted or unsubstituted tri-C₆-C₁₂arylsilyl, substituted or unsubstituted di-C₁-C₁₀ alkyl mono-C₆-C₃₀arylsilyl, and substituted or unsubstituted mono-C₁-C₁₀ alkyl di-C₆-C₃₀arylsilyl, or two adjacent groups are connected to each other to form an aliphatic ring, a heteroaromatic ring or an aromatic ring structure;
at least one of the R₁-R₁₂ has a structure as shown in a formula (2),

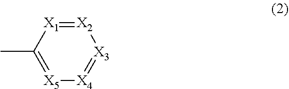

where in the formula (2),
X₁-X₅ are independently selected from N or CR₀;
in the X₁-X₅, at least one is N, and at least one is CR₀;
R₀ is independently selected from hydrogen, deuterium, halogen, cyano, hydroxyl, sulfhydryl, amino, substituted or unsubstituted C₁-C₁₀ alkyl, substituted or unsubstituted C₁-C₁₀heteroalkyl, substituted or unsubstituted C₃-C₂₀ cycloalkyl, substituted or unsubstituted C₃-C₂₀heterocycloalkyl, substituted or unsubstituted C₂-C₁₀ alkenyl, substituted or unsubstituted C₂-C₁₀ alkynyl, substituted or unsubstituted C₆-C₃₀ aryl, substituted or unsubstituted C₂-C₃₀ heteroaryl, substituted or unsubstituted tri-C₁-C₁₀alkylsilyl, substituted or unsubstituted tri-C₆-C₁₂arylsilyl, substituted or unsubstituted di-C₁-C₁₀ alkyl mono-C₆-C₃₀arylsilyl, and substituted or unsubstituted mono-C₁-C₁₀ alkyl di-C₆-C₃₀arylsilyl, or two adjacent groups are connected to each other to form an aliphatic ring, a heteroaromatic ring or an aromatic ring structure;
at least one of the R₀ has a structure as shown in a formula (3) or a formula (4),

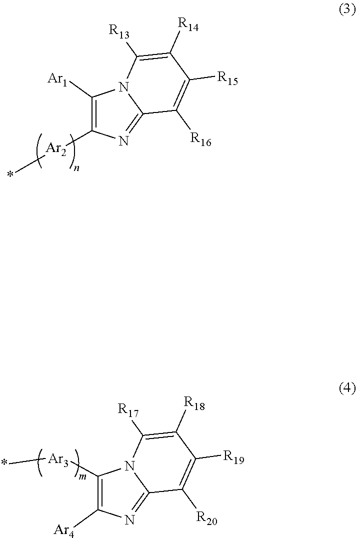

where * indicates a position connected with the formula (2);
m and n are independently selected from 0 or an integer of 1-4, and when the n or the m is 0, the formula (3) or the formula (4) is directly connected with the formula (2);
R₁₃-R₂₀ are independently selected from hydrogen, deuterium, halogen, cyano, hydroxyl, sulfhydryl, amino, substituted or unsubstituted C₁-C₁₀ alkyl, substituted or unsubstituted C₁-C₁₀heteroalkyl, substituted or unsubstituted C₃-C₂₀ cycloalkyl, substituted or unsubstituted C₃-C₂₀heterocycloalkyl, substituted or unsubstituted C₂-C₁₀ alkenyl, substituted or unsubstituted C₂-C₁₀ alkynyl, substituted or unsubstituted C₆-C₃₀aryl, substituted or unsubstituted C₂-C₃₀ heteroaryl, substituted or unsubstituted tri-C₁-C₁₀alkylsilyl, substituted or unsubstituted tri-C₆-C₁₂arylsilyl, substituted or unsubstituted di-C₁-C₁₀ alkyl mono-C₆-C₃₀arylsilyl, and substituted or unsubstituted mono-C₁-C₁₀ alkyl di-C₆-C₃₀arylsilyl, or two adjacent groups are connected to each other to form an aliphatic ring, a heteroaromatic ring or an aromatic ring structure;
Ar₁-Ar₄ are independently selected from substituted or unsubstituted C₆-C₃₀ aryl, or substituted or unsubstituted C₄-C₃₀ heteroaryl;
the heteroalkyl, the heteroaromatic ring and the heteroaryl at least contain one O, N or S heteroatom;
and the “substituted” refers to substitution with deuterium, F, Cl, Br, C₁-C₆ alkyl, C₃-C₆ cycloalkyl, C₁-C₆ alkyl substituted amino, C₆-C₁₀ aryl, cyano, isocyano, or phosphino, and the substitution ranges from a single substitution number to a maximum substitution number.

Examples of various groups of the compound represented by the formula (1) are described below.

It is to be noted that in the specification, “Cₐ-C_{b}” in the term “substituted or unsubstituted Cₐ-C_{b} X group” refers to the number of carbons when the X group is unsubstituted, excluding the number of carbons of a substituent when the X group is substituted.

As a linear or branched alkyl, the C₁-C₁₀ alkyl specifically includes methyl, ethyl, propyl, isopropyl, n-butyl, isobutyl, sec-butyl, tert-butyl, n-pentyl and isomers thereof, n-hexyl and isomers thereof, n-heptyl and isomers thereof, n-octyl and isomers thereof, n-nonyl and isomers thereof, and n-decyl and isomers thereof, optionally includes methyl, ethyl, propyl, isopropyl, n-butyl, isobutyl, sec-butyl, and tert-butyl, and more optionally includes propyl, isopropyl, isobutyl, sec-butyl, and tert-butyl.

The C₃-C₂₀ cycloalkyl may include cyclopropyl, cyclobutyl, cyclopentyl, cyclohexyl, 1-adamantyl, 2-adamantyl, 1-norbornyl, and 2-norbornyl, and optionally includes cyclopentyl and cyclohexyl.

The C₂-C₁₀ alkenyl may include vinyl, propenyl, allyl, 1-butadienyl, 2-butadienyl, 1-hexatrienyl, 2-hexatrienyl, and 3-hexatrienyl, and optionally includes propenyl and allyl.

As a linear or branched alkyl or cycloalkyl consisting of atoms other than carbon and hydrogen, the C₁-C₁₀heteroalkyl may include mercaptomethyl methyl, methoxymethyl, ethoxymethyl, tert-butoxyl methyl, N,N-dimethyl methyl, epoxy butyl, epoxy pentyl, and epoxy hexyl, and optionally includes methoxymethyl and epoxy pentyl.

Specific examples of the aryl include phenyl, naphthyl, anthracyl, phenanthryl, tetracenyl, pyrenyl, chrysenyl, benzo[c]phenanthryl, benzo[g]chrysenyl, fluorenyl, benzofluorenyl, dibenzofluorenyl, biphenyl, triphenyl, tetraphenyl, and fluoranthracyl, and optionally include phenyl and naphthyl.

Specific examples of the heteroaryl may include pyrrolyl, pyrazinyl, pyridyl, pyrimidinyl, triazinyl, indolyl, isoindolyl, imidazolyl, furyl, benzofuryl, isobenzofuryl, dibenzofuryl, dibenzothienyl, azodibenzofuryl, azodibenzothienyl, diazodibenzofuryl, diazodibenzothienyl, quinolyl, isoquinolyl, quinoxalinyl, carbazolyl, phenanthridinyl, acridinyl, phenanthrolinyl, phenazinyl, phenothiazinyl, phenoxazinyl, oxazolinyl, oxadiazolyl, furzanyl, thienyl, benzothienyl, dihydroacridinyl, azocarbazolyl, diazocarbazolyl, and quinazolinyl, and optionally include pyridyl, pyrimidinyl, triazinyl, dibenzofuryl, dibenzothienyl, azodibenzofuryl, azodibenzothienyl, diazodibenzofuryl, diazodibenzothienyl, carbazolyl, azocarbazolyl, and diazocarbazolyl.

The heteroaromatic ring has the same structure as the heteroaryl.

The following examples are merely described to facilitate the understanding of the technical invention, and should not be considered as specific limitations of the present invention.

All raw materials, solvents and the like involved in the synthesis of compounds in the present invention are purchased from Alfa, Acros, and other suppliers known to persons skilled in the art.

Synthesis of a Compound CPD002

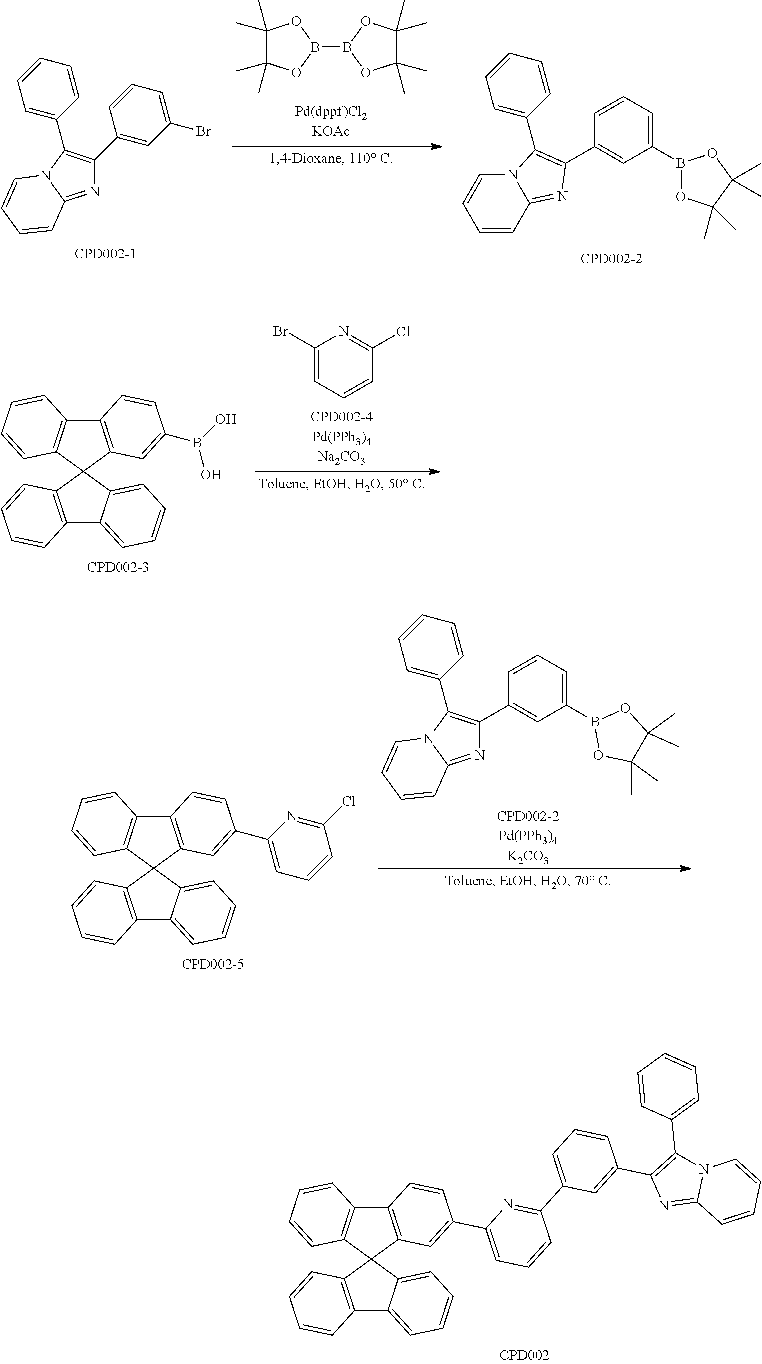

### Synthesis of a Compound CPD002-2

CPD002-1 (25.00 g, 71.59 mmol), bis(pinacolato)diboron (23.63 g, 93.06 mmol), [1,1′-bis(diphenylphosphino)ferrocene]dichloropalladium (2.60 g, 3.58 mmol), potassium acetate (14.05 g, 143.18 mmol) and 1,4-dioxane (250 ml) were added into a 1,000 ml three-neck round-bottomed flask, subjected to nitrogen replacement under vacuum for three times, and heated to 110° C. to carry out a reaction for 4 h. According to monitoring by thin layer chromatography (TLC, with a mixture of dichloromethane and methanol at a ratio of 40:1 as a developing agent), the reaction was stopped when the CPD002-1 was completely consumed.

A reaction solution was concentrated under reduced pressure, ethyl acetate (1,000 ml) was added, and washing was performed with deionized water (3*300 ml). Then, an organic phase was subjected to silica gel column chromatography (with 200- to 300-mesh silica gel and a mixture of dichloromethane and methanol at a ratio of 70:1 as an eluting agent), followed by elution and concentration to obtain a light yellow solid compound CPD002-2 (22.79 g, purity: 98.01%, yield: 80.34%). Mass spectrometry was performed at 397.20 (M+H).

### Synthesis of a Compound CPD002-5

CPD002-3 (25.00 g, 69.40 mmol), CPD002-4 (14.69 g, 76.34 mmol), tetrakis(triphenylphosphin)palladium (1.60 g, 1.39 mmol), sodium carbonate (14.71 g, 138.80 mmol), toluene (375 ml), ethanol (125 ml) and deionized water (125 ml) were added into a 1,000 ml three-neck round-bottomed flask, subjected to nitrogen replacement under vacuum for three times, and heated to 50° C. to carry out a reaction for 3 h. According to monitoring by TLC (with a mixture of ethyl acetate and petroleum ether at a ratio of 1:10 as a developing agent), the reaction was stopped when the raw material CPD002-3 was completely consumed.

Toluene (500 ml) was added, washing was performed with deionized water (3*300 ml), and extraction was performed for liquid separation. Then, silica gel column chromatography was performed (with 200- to 300-mesh silica gel and a mixture of ethyl acetate and petroleum ether at a ratio of 1:50 as an eluting agent), followed by elution and concentration to obtain a light yellow solid compound CPD002-5 (22.38 g, purity: 99.73%, yield: 75.35%). Mass spectrometry was performed at 428.21 (M+H).

### Synthesis of a Compound CPD002

The CPD002-5 (20.00 g, 46.74 mmol), the CPD002-2 (20.37 g, 51.41 mmol), tetrakis(triphenylphosphin)palladium (1.08 g, 0.94 mmol), potassium carbonate (12.92 g, 93.48 mmol), toluene (300 ml), ethanol (100 ml) and deionized water (100 ml) were added into a 1,000 ml three-neck round-bottomed flask, subjected to nitrogen replacement under vacuum for three times, and heated to 80° C. to carry out a reaction for 5 h. According to monitoring by TLC (with a mixture of ethyl acetate and petroleum ether at a ratio of 1:5 as a developing agent), the reaction was stopped when the raw material CPD002-5 was completely consumed.

Toluene (500 ml) was added, washing was performed with deionized water (3*300 ml), and extraction was performed for liquid separation. Then, silica gel column chromatography was performed (with 200- to 300-mesh silica gel and a mixture of ethyl acetate and petroleum ether at a ratio of 1:10 as an eluting agent), followed by elution and concentration to obtain a light yellow solid compound CPD002 (26.40 g, purity: 99.95%, yield: 85.34%). 26.40 g of the crude product CPD002 was sublimated and purified to obtain sublimated and purified CPD002 (17.24 g, purity: 99.96%, yield: 65.30%). Mass spectrometry was performed at 662.82 (M+H).

¹H NMR (400 MHz, CDCl₃) δ 8.82-8.74 (m, 2H), 8.67-8.63 (m, 1H), 8.57-8.55 (m, 1H), 8.45-8.35 (m, 2H), 8.28-8.14 (m, 6H), 8.02-7.94 (m, 2H), 7.93-7.76 (m, 6H), 7.70-7.63 (m, 2H), 7.61-7.53 (m, 5H), 7.50-7.41 (m, 1H), 7.21-7.15 (m, 1H), 7.08-7.05 (m, 2H).

### Synthesis of a Compound CPD009

### Synthesis of a Compound CPD009-2

With reference to the synthesis and purification methods of the compound CPD002-2, only the corresponding raw materials were required to be changed, and a target compound CPD009-2 (20.87 g, purity: 98.11%, yield: 75.01%) was obtained. Mass spectrometry was performed at 397.20 (M+H).

### Synthesis of a Compound CPD009-5

CPD002-3 (25.00 g, 69.40 mmol), CPD009-4 (16.42 g, 76.34 mmol), tetrakis(triphenylphosphin)palladium (1.60 g, 1.39 mmol), potassium carbonate (19.18 g, 138.80 mmol), toluene (375 ml), ethanol (125 ml) and deionized water (125 ml) were added into a 2,000 ml three-neck round-bottomed flask, subjected to nitrogen replacement under vacuum for three times, and heated to 75° C. to carry out a reaction for 8 h. According to monitoring by TLC (with a mixture of ethyl acetate and petroleum ether at a ratio of 1:8 as a developing agent), the reaction was stopped when the raw material CPD002-3 was completely consumed.

Toluene (600 ml) was added, washing was performed with deionized water (3*350 ml), and extraction was performed for liquid separation. Then, silica gel column chromatography was performed (with 200- to 300-mesh silica gel and a mixture of ethyl acetate and petroleum ether at a ratio of 1:20 as an eluting agent), followed by elution and concentration to obtain a yellow solid compound CPD009-5 (20.02 g, purity: 99.51%, yield: 60.23%). Mass spectrometry was performed at 479.32 (M+H).

### Synthesis of a Compound CPD009

The CPD009-5 (15.00 g, 31.32 mmol), the CPD009-2 (13.65 g, 34.45 mmol), dichlorobis[di-tert-butyl(4-dimethylaminophenyl)phosphino]palladium (0.22 g, 0.32 mmol), potassium carbonate (6.65 g, 62.60 mmol), 1,4-dioxane (150 ml) and deionized water (30 ml) were added into a 500 ml three-neck round-bottomed flask, subjected to nitrogen replacement under vacuum for three times, and heated to 100° C. to carry out a reaction for 2 h. According to monitoring by TLC (with a mixture of ethyl acetate and petroleum ether at a ratio of 1:5 as a developing agent), the reaction was stopped when the raw material CPD009-5 was completely consumed.

An organic phase was removed by concentration under reduced pressure, ethyl acetate (800 ml) was added, washing was performed with deionized water (3*250 ml), and extraction was performed for liquid separation. Then, silica gel column chromatography was performed (with 200- to 300-mesh silica gel and a mixture of ethyl acetate and petroleum ether at a ratio of 1:10 as an eluting agent), followed by elution and concentration to obtain a light yellow solid compound CPD009 (19.47 g, purity: 99.93%, yield: 87.21%). 19.47 g of the crude product CPD009 was sublimated and purified to obtain sublimated and purified CPD009 (12.29 g, purity: 99.94%, yield: 63.12%). Mass spectrometry was performed at 713.14 (M+H).

¹H NMR (400 MHz, CDCl₃) δ 8.81-8.77 (m, 1H), 8.65-8.59 (m, 2H), 8.46-8.42 (m, 1H), 8.39 (d, J=15.0 Hz, 1H), 8.32 (d, J=2.9 Hz, 1H), 8.30-8.25 (m, 2H), 8.23-8.21 (m, 2H), 8.21-8.13 (m, 4H), 8.10-8.07 (m, 1H), 8.02 7.97 (m, 3H), 7.94-7.77 (m, 6H), 7.65 (td, J=14.8, 3.3 Hz, 2H), 7.60-7.47 (m, 5H), 7.20-7.13 (m, 1H).

### Synthesis of a Compound CPD014

### Synthesis of a Compound CPD014-2

With reference to the synthesis and purification methods of the compound CPD002-2, only the corresponding raw materials were required to be changed, and a target compound CPD014-2 (26.37 g, purity: 98.00%, yield: 72.11%) was obtained. Mass spectrometry was performed at 397.20 (M+H).

### Synthesis of a Compound CPD014

With reference to the synthesis and purification methods of the compound CPD009, only the corresponding raw materials were required to be changed, and a target compound CPD014 (12.54 g, purity: 99.96%, yield: 82.41%) was obtained. 12.54 g of the crude product CPD014 was sublimated and purified to obtain sublimated and purified CPD014 (7.36 g, purity: 99.94%, yield: 58.69%). Mass spectrometry was performed at 713.14 (M+H).

¹H NMR (400 MHz, CDCl₃) δ 8.82-8.78 (m, 1H), 8.64-8.58 (m, 2H), 8.44 (d, 3.1 Hz, 1H), 8.39 (d, J=15.0 Hz, 1H), 8.32 (d, J=3.1 Hz, 1H), 8.30-8.17 (m, 6H), 8.14-8.07 (m, 1H), 8.05-7.97 (m, 3H), 7.95-7.85 (m, 2H), 7.84-7.74 (m, 4H), 7.71-7.61 (m, 3H), 7.61-7.47 (m, 6H), 7.20-7.14 (m, 1H).

### Synthesis of a Compound CPD017

### Synthesis of a Compound CPD017-5

With reference to the synthesis and purification methods of the compound CPD009-5, only the corresponding raw materials were required to be changed, and a target compound CPD017-5 (21.01 g, purity: 99.61%, yield: 65.32%) was obtained. Mass spectrometry was performed at 433.12 (M+H).

### Synthesis of a Compound CPD017

With reference to the synthesis and purification methods of the compound CPD009, only the corresponding raw materials were required to be changed, and a target compound CPD017 (15.66 g, purity: 99.92%, yield: 77.54%) was obtained. 15.66 g of the crude product CPD017 was sublimated and purified to obtain sublimated and purified CPD017 (9.51 g, purity: 99.92%, yield: 60.72%). Mass spectrometry was performed at 713.14 (M+H).

¹H NMR (400 MHz, CDCl₃) δ 8.80-7.78 (m, 1H), 8.62-8.59 (m, 4H), 8.49-8.37 (m, 3H), 8.30-8.27 (m, 1H), 8.26-8.09 (m, 6H), 8.00-7.98 (m, 1H), 7.95-7.79 (m, 8H), 7.70-7.67 (m, 2H), 7.61-7.47 (m, 5H), 7.21-7.15 (m, 1H).

### Synthesis of a Compound CPD026

### Synthesis of a Compound CPD026-2

With reference to the synthesis and purification methods of the compound CPD002-2, only the corresponding raw materials were required to be changed, and a target compound CPD026-2 (35.86 g, purity: 98.11%, yield: 70.01%) was obtained. Mass spectrometry was performed at 397.20 (M+H).

### Synthesis of a Compound CPD026-5

CPD026-3 (25.00 g, 93.44 mmol) and dry tetrahydrofuran (375 ml) were added into a 1,000 ml three-neck round-bottomed flask, subjected to nitrogen replacement for three times and cooled to −78° C., and an n-hexane solution containing 2.5 mol/l of n-butyllithium (37.38 ml, 93.44 mmol) was added dropwise and completely dropped within 1 h to carry out a thermal insulation reaction at −78° C. for 1 h. The system was heated to −50° C. until being changed into clarified liquid, CPD026-4 (22.22 g, 102.78 mmol) was directly added, and the system was heated to −30° C. until being turned into brownish red and then slowly heated to room temperature to carry out a stirring reaction overnight. According to monitoring of the reaction by TLC (with a mixture of ethyl acetate and n-hexane at a ratio of 1:40 as a developing agent), the raw material CPD026-3 was completely consumed.

A saturated ammonium chloride aqueous solution (250 ml) was added for quenching the reaction, the temperature was gradually raised to room temperature, the tetrahydrofuran was removed by concentration, ethyl acetate (800 ml) was added, and washing was performed with deionized water (3*300 ml). Then, purification was performed by silica gel column chromatography (with 200- to 300-mesh silica gel and a mixture of tetrahydrofuran and petroleum ether at a ratio of 1:20 as an eluting agent), followed by concentration to obtain a white-like solid compound CPD026-5 (24.72 g, purity: 99.20%, yield: 65.35%). Mass spectrometry was performed at 405.08 (M+H).

### Synthesis of a Compound CPD026-6

The CPD026-5 (23.50 g, 58.05 mmol), acetic acid (240 ml) and 36%-38% concentrated hydrochloric acid (12 ml) were added into a 1,000 ml one-neck round-bottomed flask and heated to 90° C. to carry out a stirring reaction for 2 h. According to monitoring by TLC (with a mixture of ethyl acetate and petroleum ether at a ratio of 1:40 as a developing agent), the raw material CPD026-5 was completely consumed.

The temperature was lowered to 60° C., ethanol (250 ml) was added, and suction filtration was performed to obtain a white-like solid. The solid was recrystallized with toluene and methanol for two times, followed by suction filtration and drying to obtain a white-like solid compound CPD026-6 (16.98 g, purity: 99.85%, yield: 75.62%). Mass spectrometry was performed at 387.27 (M+H).

### Synthesis of a Compound CPD026-7

The CPD026-6 (15 g, 38.77 mmol), bis(pinacolato)diboron (16.69 g, 77.55 mmol), tris(dibenzylideneacetone)dipalladium (0.714 g, 0.78 mmol), potassium acetate (7.61 g, 77.54 mmol) and 1,4-dioxane (150 ml) were added into a 500 ml three-neck round-bottomed flask, subjected to nitrogen replacement under vacuum for three times, and heated to 110° C. to carry out a reaction for 2 h. According to monitoring by TLC (with a mixture of ethyl acetate and petroleum ether at a ratio of 1:20), the reaction was stopped when the CPD026-6 was completely consumed.

A reaction solution was concentrated under reduced pressure, ethyl acetate (900 ml) was added, and washing was performed with deionized water (3*300 ml). Then, an organic phase was subjected to silica gel column chromatography (with 200- to 300-mesh silica gel and a mixture of ethyl acetate and petroleum ether at a ratio of 1:40 as an eluting agent), followed by elution and concentration to obtain a white solid compound, namely a target compound CPD026-7 (15.82 g, purity: 98.36%, yield: 85.31%). Mass spectrometry was performed at 479.20 (M+H).

### Synthesis of a Compound CPD026-8

With reference to the synthesis and purification methods of the compound CPD002-5, only the corresponding raw materials were required to be changed, and a target compound CPD026-8 (17.54 g, purity: 99.62%, yield: 64.23%) was obtained. Mass spectrometry was performed at 433.21 (M+H).

### Synthesis of a Compound CPD026

With reference to the synthesis and purification methods of the compound CPD009, only the corresponding raw materials were required to be changed, and a target compound CPD026 (22.86 g, purity: 99.92%, yield: 80.14%) was obtained. 22.86 g of the crude product CPD026 was sublimated and purified to obtain sublimated and purified CPD026 (13.47 g, purity: 99.92%, yield: 58.92%). Mass spectrometry was performed at 749.26 (M+H).

¹H NMR (400 MHz, CDCl₃) δ 8.83-8.79 (m, 1H), 8.50-8.44 (m, 2H), 8.43-8.40 (m, 2H), 8.37-8.33 (m, 1H), 8.29 (dd, J=2.9, 0.9 Hz, 1H), 8.27-8.25 (m, 1H), 8.24-8.16 (m, 3H), 8.15-8.09 (m, 1H), 8.07-7.97 (m, 2H), 7.94-7.71 (m, 8H), 7.66-7.49 (m, 7H), 7.12-7.16 (m, 1H).

### Synthesis of a Compound CPD027

### Synthesis of a Compound CPD027-5

With reference to the synthesis and purification methods of the compound CPD009-5, only the corresponding raw materials were required to be changed, and a target compound CPD027-5 (18.69 g, purity: 99.63%, yield: 61.25%) was obtained. Mass spectrometry was performed at 505.26 (M+H).

### Synthesis of a Compound CPD027

With reference to the synthesis and purification methods of the compound CPD009, only the corresponding raw materials were required to be changed, and a target compound CPD027 (18.02 g, purity: 99.95%, yield: 78.26%) was obtained. 18.02 g of the crude product CPD027 was sublimated and purified to obtain sublimated and purified CPD027 (11.16 g, purity: 99.96%, yield: 61.93%). Mass spectrometry was performed at 739.20 (M+H).

¹H NMR (400 MHz, CDCl₃) δ 8.81-8.77 (m, 1H), 8.64-8.58 (m, 2H), 8.54 (s, 1H), 8.41-8.35 (m, 2H), 8.30-8.11 (m, 9H), 8.01-7.97 (m, 1H), 7.96-7.78 (m, 10H), 7.69-7.32 (m, 2H), 7.60-7.47 (m, 5H), 7.20-7.14 (m, 1H).

### Synthesis of a Compound CPD033

### Synthesis of a Compound CPD033

With reference to the synthesis and purification methods of the compound CPD009, only the corresponding raw materials were required to be changed, and a target compound CPD033 (22.34 g, purity: 99.97%, yield: 78.41%) was obtained. 22.34 g of the crude product CPD033 was sublimated and purified to obtain sublimated and purified CPD033 (14.47 g, purity: 99.98%, yield: 64.77%). Mass spectrometry was performed at 739.20 (M+H).

¹H NMR (400 MHz, CDCl₃) δ 8.80-8.78 (m, 1H), 8.72-8.68 (m, 1H), 8.55 (s, 1H), 8.45 (t, J=2.9 Hz, 1H), 8.42-8.39 (m, 1H), 8.35 (d, J=2.9 Hz, 1H), 8.24-8.19 (m, 6H), 8.07-7.97 (m, 2H), 7.97-7.74 (m, 10H), 7.72-7.48 (m, 9H), 7.21-7.15 (m, 1H).

### Synthesis of a Compound CPD041

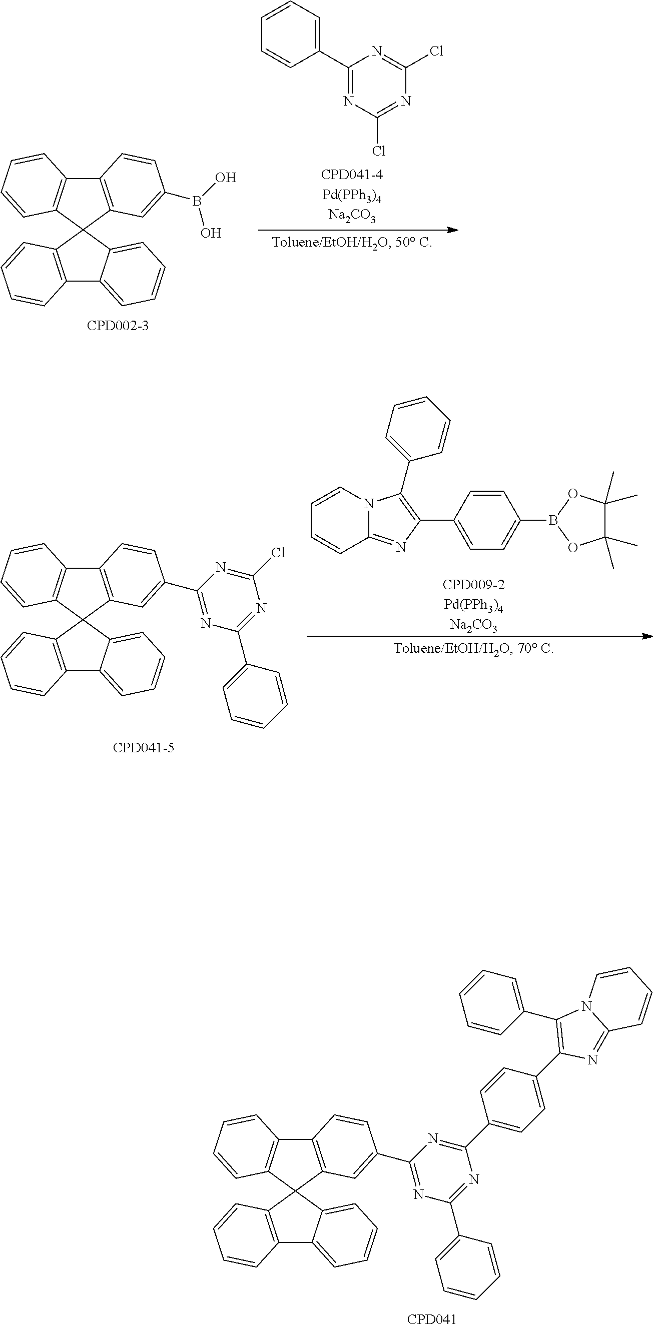

### Synthesis of a Compound CPD041-5

CPD002-3 (21.68 g, 60.19 mmol), CPD041-4 (14.97 g, 66.20 mmol), sodium carbonate (12.76 g, 120.38 mmol), tetrakis(triphenylphosphin)palladium (1.39 g, 1.21 mmol), toluene (100 ml), ethanol (60 ml) and deionized water (60 ml) were added into a 1,000 ml three-neck round-bottomed flask, subjected to nitrogen replacement under vacuum for three times, and heated to 50° C. to carry out a reaction for 3 h. According to monitoring by TLC (with a mixture of ethyl acetate and petroleum ether at a ratio of 1:10 as a developing agent), the heating was stopped when the CPD002-3 was completely consumed.

A reaction solution was concentrated under reduced pressure, ethyl acetate (600 ml) was added, and washing was performed with deionized water (3*200 ml). Then, silica gel column chromatography was performed (with 200- to 300-mesh silica gel and a mixture of ethyl acetate and petroleum ether at a ratio of 1:30 as an eluting agent), followed by elution and concentration to obtain a white solid compound CPD041-5 (27.70 g, purity: 99.61%, yield: 81.12%). Mass spectrometry was performed at 506.23 (M+H).

### Synthesis of a Compound CPD041

The CPD041-5 (26.71 g, 52.79 mmol), CPD009-2 (22.20 g, 58.06 mmol), sodium carbonate (11.19 g, 105.58 mmol), tetrakis(triphenylphosphin)palladium (1.22 g, 1.06 mmol), toluene (270 ml), ethanol (60 ml) and deionized water (60 ml) were added into a 1,000 ml three-neck round-bottomed flask, subjected to nitrogen replacement under vacuum for three times, and heated to 70° C. to carry out a reaction for 3 h. According to monitoring by TLC (with a mixture of ethyl acetate and petroleum ether at a ratio of 1:8 as a developing agent), the heating was stopped when the CPD041-5 was completely consumed.

A reaction solution was concentrated under reduced pressure, dichloromethane (1,000 ml) was added, and washing was performed with deionized water (3*450 ml). Then, silica gel column chromatography was performed (with 200- to 300-mesh silica gel and a mixture of ethyl acetate and petroleum ether at a ratio of 1:20 as an eluting agent), followed by elution and concentration to obtain a white solid compound CPD041 (32.71 g, purity: 99.96%, yield: 83.76%). 32.71 g of the crude product CPD041 was sublimated and purified to obtain sublimated and purified CPD041 (20.74 g, purity: 99.96%, yield: 63.40%). Mass spectrometry was performed at 740.17 (M+H).

¹H NMR (400 MHz, CDCl₃) δ 8.82-8.80 (m, 1H), 8.72-8.66 (m, 2H), 8.63 (d, J=7.5 Hz, 2H), 8.51 (d, J=1.4 Hz, 1H), 8.41 (d, J=7.5 Hz, 1H), 8.29 (d, J=7.3 Hz, 2H), 8.24-8.21 (m, 3H), 8.20-8.14 (m, 2H), 8.01 (dd, J=7.5, 1.4 Hz, 1H), 7.98-7.91 (m, 3H), 7.89-7.79 (m, 7H), 7.67 (td, J=7.5, 1.5 Hz, 2H), 7.62-7.48 (m, 5H), 7.21-7.19 (m, 1H).

### Synthesis of a Compound CPD046

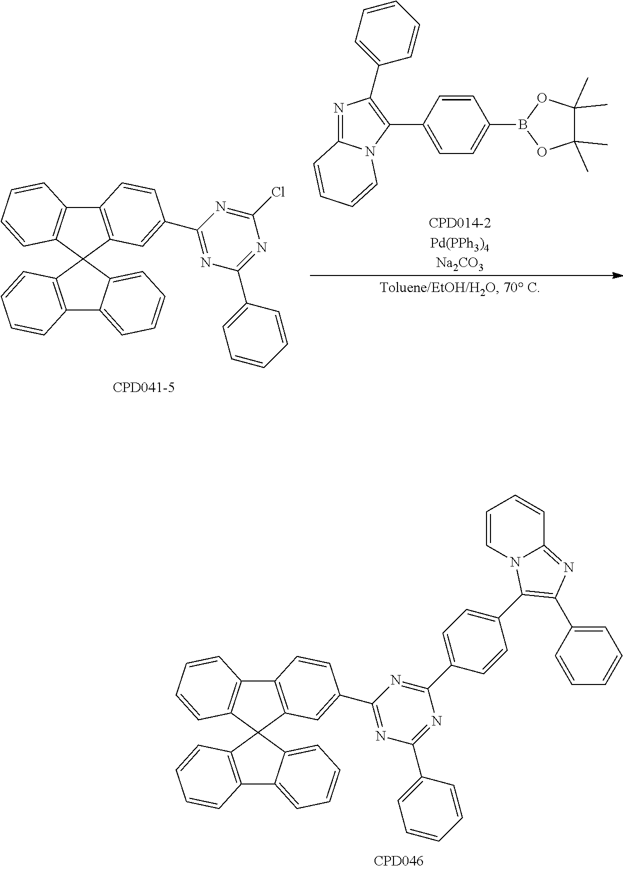

With reference to the synthesis and purification methods of the compound CPD041, only the corresponding raw materials were required to be changed, and a target compound CPD046 (28.73 g, purity: 99.93%, yield: 75.26%) was obtained. 28.73 g of the crude product CPD046 was sublimated and purified to obtain sublimated and purified CPD046 (18.46 g, purity: 99.94%, yield: 64.25%). Mass spectrometry was performed at 740.17 (M+H).

¹H NMR (400 MHz, CDCl₃) δ 8.82-8.80 (m, 1H), 8.72-8.66 (m, 2H), 8.63 (d, J=7.5 Hz, 2H), 8.53 (d, J=1.4 Hz, 1H), 8.41 (d, J=7.5 Hz, 1H), 8.29 (d, J=7.3 Hz, 2H), 8.24-8.21 (m, 3H), 8.01 (dd, J=7.5, 1.4 Hz, 1H), 7.99-7.91 (m, 3H), 7.88-7.77 (m, 7H), 7.67 (td, J=7.5, 1.5 Hz, 2H), 7.64-7.51 (m, 7H), 7.21-7.17 (m, 1H).

### Synthesis of a Compound CPD054

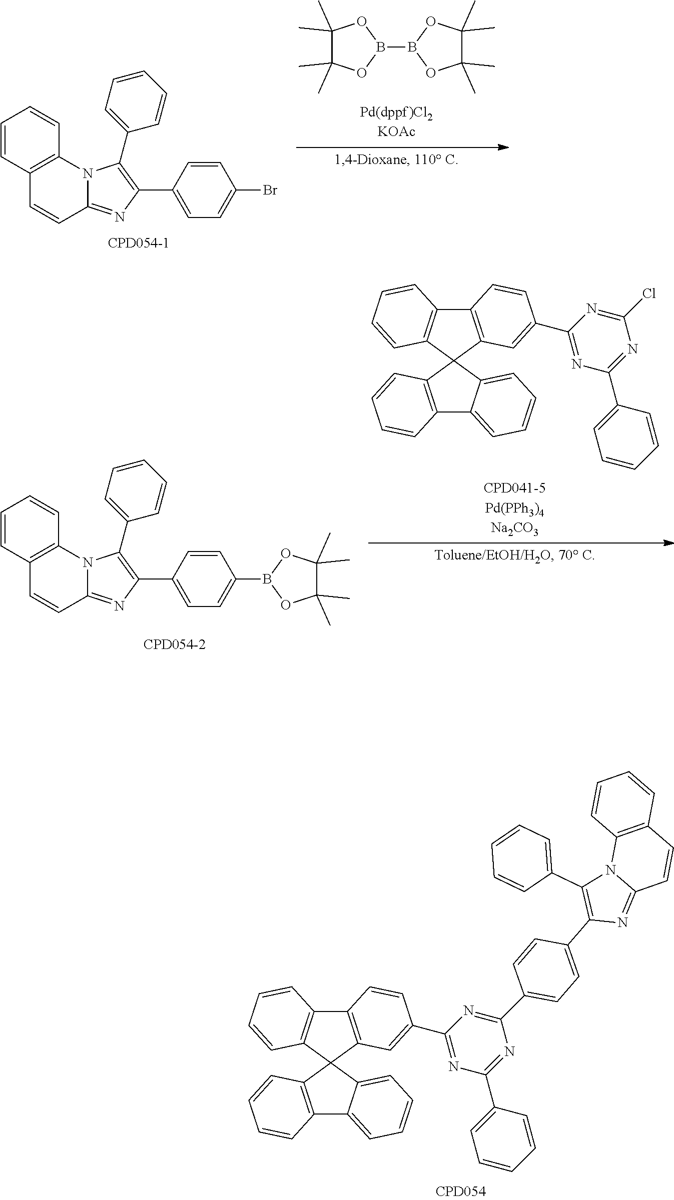

### Synthesis of a Compound CPD054-2

With reference to the synthesis and purification methods of the compound CPD002-2, only the corresponding raw materials were required to be changed, and a target compound CPD054-2 (24.36 g, purity: 98.65%, yield: 68.88%) was obtained. Mass spectrometry was performed at 447.21 (M+H).

### Synthesis of a Compound CPD054

With reference to the synthesis and purification methods of the compound CPD041, only the corresponding raw materials were required to be changed, and a target compound CPD054 (21.12 g, purity: 99.92%, yield: 68.72%) was obtained. 21.12 g of the crude product CPD054 was sublimated and purified to obtain sublimated and purified CPD054 (12.58 g, purity: 99.94%, yield: 59.56%). Mass spectrometry was performed at 790.40 (M+H).

¹H NMR (400 MHz, CDCl₃) δ 8.83-8.79 (m, 1H), 8.61-8.45 (m, 5H), 8.45-8.36 (m, 2H), 8.27 (d, J=15.0 Hz, 1H), 8.24-7.91 (m, 10H), 7.91-7.77 (m, 4H), 7.77-7.63 (m, 6H), 7.54 (td, J=14.9, 3.4 Hz, 2H), 7.48-7.36 (m, 4H).

### Synthesis of a Compound CPD059

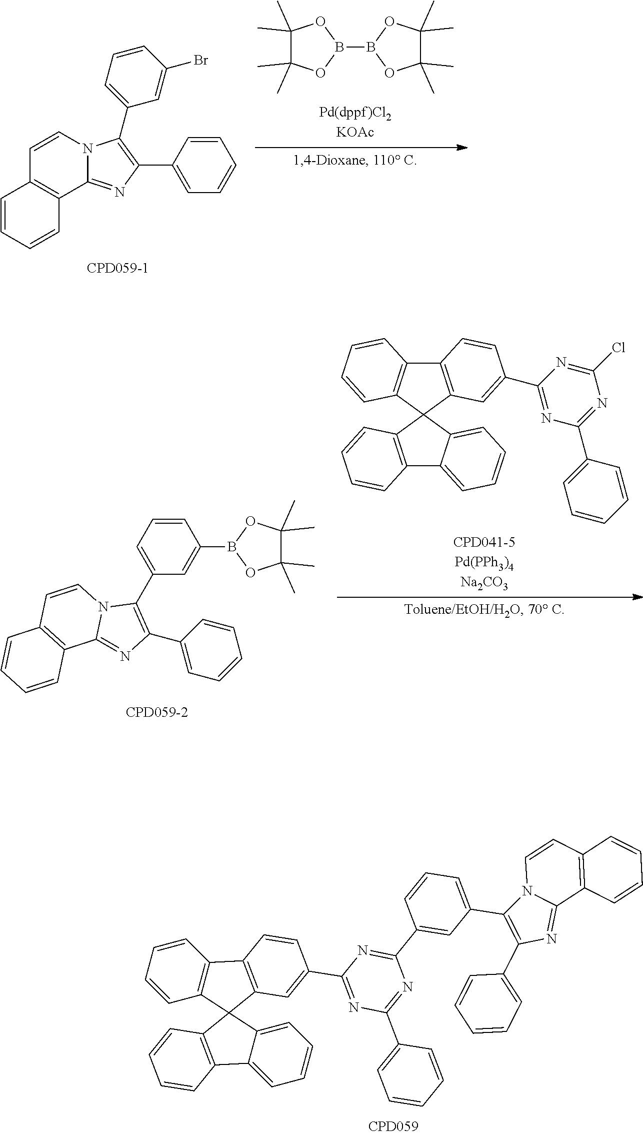

### Synthesis of a Compound CPD059-2

With reference to the synthesis and purification methods of the compound CPD002-2, only the corresponding raw materials were required to be changed, and a target compound CPD059-2 (20.93 g, purity: 98.83%, yield: 71.11%) was obtained. Mass spectrometry was performed at 447.21 (M+H).

### Synthesis of a Compound CPD059

With reference to the synthesis and purification methods of the compound CPD041, only the corresponding raw materials were required to be changed, and a target compound CPD059 (18.94 g, purity: 99.93%, yield: 69.79%) was obtained. 18.94 g of the crude product CPD059 was sublimated and purified to obtain sublimated and purified CPD059 (10.40 g, purity: 99.93%, yield: 54.91%). Mass spectrometry was performed at 790.40 (M+H).

¹H NMR (400 MHz, CDCl₃) δ 8.79 (d, J=15.0 Hz, 1H), 7.80-7.66 (m, 4H), 7.55 (d, J=2.9 Hz, 1H), 7.45 (d, J=15.0 Hz, 1H), 7.35-7.21 (m, 5H), 7.18-6.92 (m, 9H), 6.92-6.79 (m, 6H), 6.78-6.66 (m, 3H), 6.66-6.54 (m, 5H).

### Synthesis of a Compound CPD065

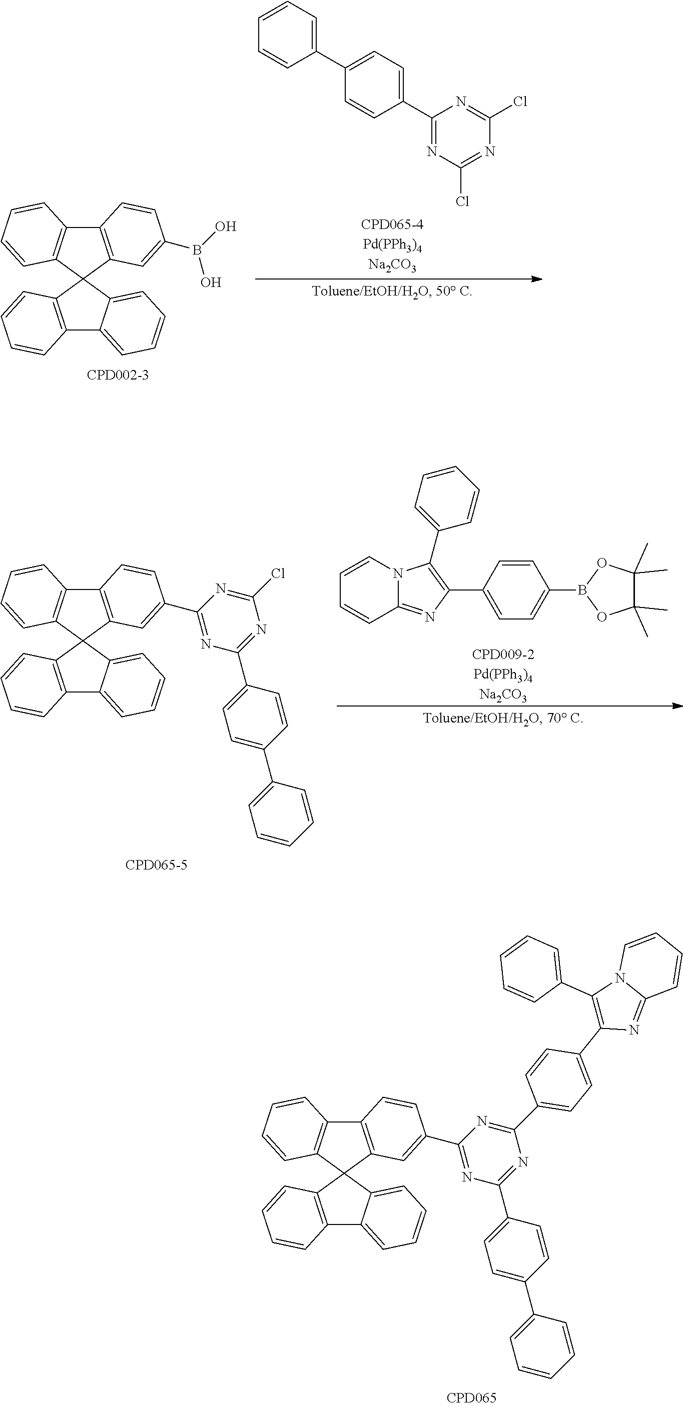 [SMILES: Clc1nc(Cl)nc(-c2ccc(-c3ccccc3)cc2)n1 ; OB(O)c1ccc2c(c1)C1(c3ccccc3-c3ccccc31)c1ccccc1-2 ; Clc1nc(-c2ccc(-c3ccccc3)cc2)nc(-c2ccc3c(c2)C2(c4ccccc4-c4ccccc42)c2ccccc2-3)n1 ; CC1(C)OB(c2ccc(-c3nc4ccccn4c3-c3ccccc3)cc2)OC1(C)C ; "CPD009-2Pd(PPh3)4Na2CO3" ; c1ccc(-c2ccc(-c3nc(-c4ccc(-c5nc6ccccn6c5-c5ccccc5)cc4)nc(-c4ccc5c(c4)C4(c6ccccc6-c6ccccc64)c4ccccc4-5)n3)cc2)cc1]

### Synthesis of a Compound CPD065-5

With reference to the synthesis and purification methods of the compound CPD041-5, only the corresponding raw materials were required to be changed, and a target compound CPD065-5 (24.36 g, purity: 99.35%, yield: 65.87%) was obtained. Mass spectrometry was performed at 582.26 (M+H).

### Synthesis of a Compound CPD065

With reference to the synthesis and purification methods of the compound CPD041, only the corresponding raw materials were required to be changed, and a target compound CPD065 (20.01 g, purity: 99.95%, yield: 76.96%) was obtained. 20.01 g of the crude product CPD065 was sublimated and purified to obtain sublimated and purified CPD065 (11.78 g, purity: 99.95%, yield: 58.87%). Mass spectrometry was performed at 816.32 (M+H).

¹H NMR (400 MHz, CDCl₃) δ 8.82-8.78 (m, 1H), 8.67-8.59 (m, 2H), 8.52 (d, J=3.0 Hz, 1H), 8.40 (d, J=15.0 Hz, 1H), 8.33-8.11 (m, 9H), 8.11-7.89 (m, 6H), 7.89-7.62 (m, 9H), 7.62-7.47 (m, 7H), 7.21-7.15 (m, 1H).

### Synthesis of a Compound CPD072

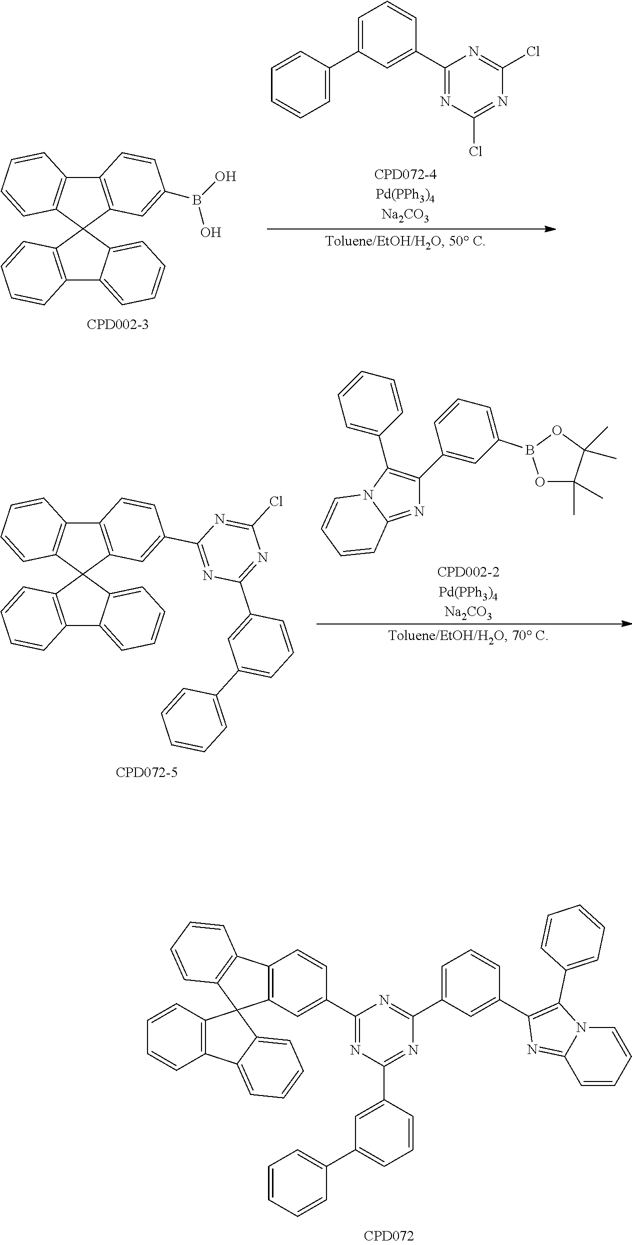 [SMILES: Clc1nc(Cl)nc(-c2cccc(-c3ccccc3)c2)n1 ; OB(O)c1ccc2c(c1)C1(c3ccccc3-c3ccccc31)c1ccccc1-2 ; Clc1nc(-c2cccc(-c3ccccc3)c2)nc(-c2ccc3c(c2)C2(c4ccccc4-c4ccccc42)c2ccccc2-3)n1 ; CC1(C)OB(c2cccc(-c3nc4ccccn4c3-c3ccccc3)c2)OC1(C)C ; "CPD002-2Pd(PPh3)4Na2CO3" ; c1ccc(-c2cccc(-c3nc(-c4cccc(-c5nc6ccccn6c5-c5ccccc5)c4)nc(-c4ccc5c(c4)C4(c6ccccc6-c6ccccc64)c4ccccc4-5)n3)c2)cc1]

### Synthesis of a Compound CPD072-5

With reference to the synthesis and purification methods of the compound CPD041-5, only the corresponding raw materials were required to be changed, and a target compound CPD072-5 (23.09 g, purity: 99.61%, yield: 68.45%) was obtained. Mass spectrometry was performed at 582.26 (M+H).

### Synthesis of a Compound CPD072

With reference to the synthesis and purification methods of the compound CPD041, only the corresponding raw materials were required to be changed, and a target compound CPD072 (30.84 g, purity: 99.96%, yield: 82.39%) was obtained. 30.84 g of the crude product CPD072 was sublimated and purified to obtain sublimated and purified CPD072 (19.29 g, purity: 99.95%, yield: 62.54%). Mass spectrometry was performed at 816.32 (M+H).

¹H NMR (400 MHz, CDCl₃) δ 8.80-8.78 (m, 1H), 8.74-8.71 (m, 2H), 8.69 (t, J=3.1 Hz, 1H), 8.58 (t, J=3.0 Hz, 1H), 8.52 (d, J=3.1 Hz, 1H), 8.40 (d, J=15.0 Hz, 1H), 8.32-8.11 (m, 6H), 8.12-7.90 (m, 9H), 7.90-7.71 (m, 7H), 7.71-7.62 (m, 2H), 7.62-7.48 (m, 5H), 7.21-7.15 (m, 1H).

### Synthesis of a Compound CPD074

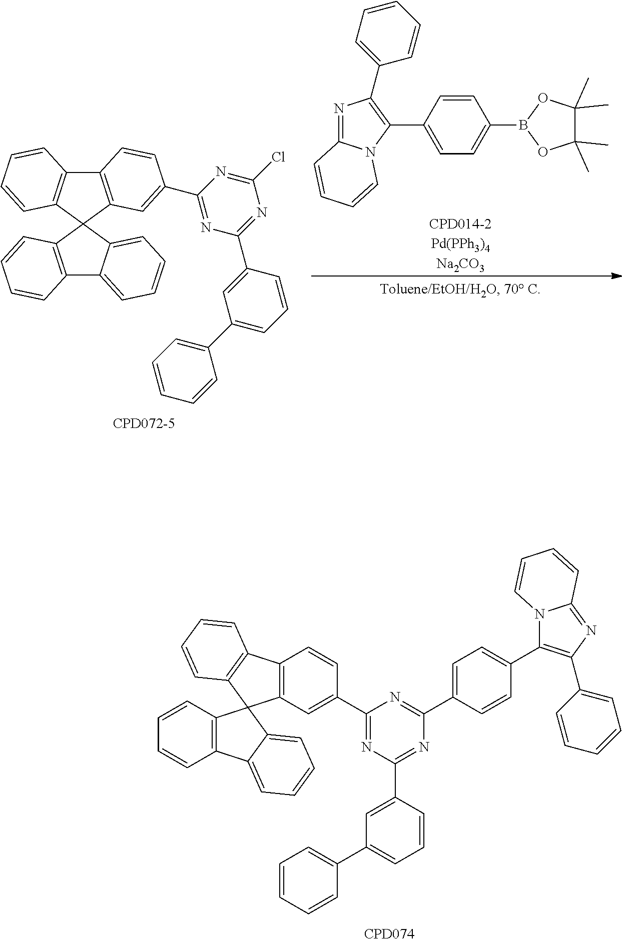

With reference to the synthesis and purification methods of the compound CPD041, only the corresponding raw materials were required to be changed, and a target compound CPD074 (27.47 g, purity: 99.95%, yield: 79.85%) was obtained. 27.47 g of the crude product CPD074 was sublimated and purified to obtain sublimated and purified CPD074 (15.79 g, purity: 99.95%, yield: 57.48%). Mass spectrometry was performed at 816.32 (M+H).

¹H NMR (400 MHz, CDCl₃) δ 8.81-8.77 (m, 1H), 8.70 (dt, J=14.7, 3.2 Hz, 1H), 8.66-8.58 (m, 2H), 8.57 (t, J=2.9 Hz, 1H), 8.52 (d, J=2.9 Hz, 1H), 8.40 (d, J=15.0 Hz, 1H), 8.33-8.15 (m, 5H), 8.12-7.90 (m, 8H), 7.89-7.72 (m, 7H), 7.72-7.62 (m, 3H), 7.62-7.47 (m, 6H), 7.22-7.16 (m, 1H).

### Synthesis of a Compound CPD075

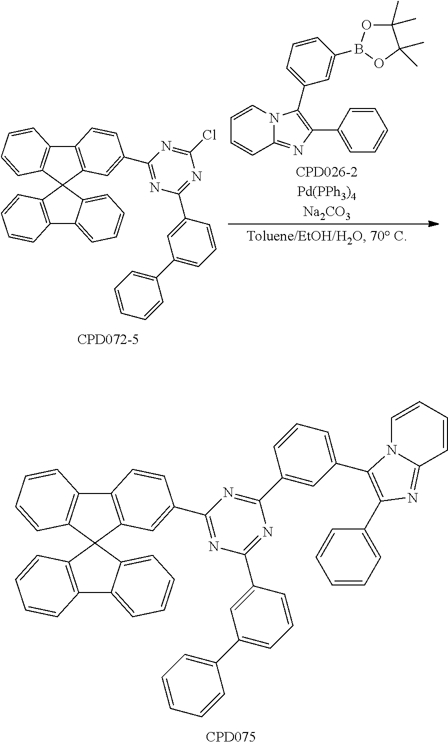

With reference to the synthesis and purification methods of the compound CPD041, only the corresponding raw materials were required to be changed, and a target compound CPD075 (23.21 g, purity: 99.96%, yield: 76.25%) was obtained. 23.21 g of the crude product CPD075 was sublimated and purified to obtain sublimated and purified CPD075 (13.59 g, purity: 99.95%, yield: 58.55%). Mass spectrometry was performed at 816.32 (M+H).

¹H NMR (400 MHz, CDCl₃) δ 8.83-8.81 (m, 3H), 8.74 (d, J=7.5 Hz, 1H), 8.57 (d, J=7.6 Hz, 1H), 8.08 (s, 1H), 8.04 (d, J=7.8 Hz, 2H), 7.96 (d, J=7.6 Hz, 1H), 7.88 (d, J=7.6 Hz, 2H), 7.80-7.75 (m, 2H), 7.72-7.56 (m, 7H), 7.50 (t, J=7.4 Hz, 2H), 7.44-7.36 (m, 4H), 7.29-7.26 (m, 3H), 7.23-7.16 (m, 2H), 7.12 (t, J=7.5 Hz, 2H), 6.79-6.73 (m, 4H).

### Synthesis of a Compound CPD077

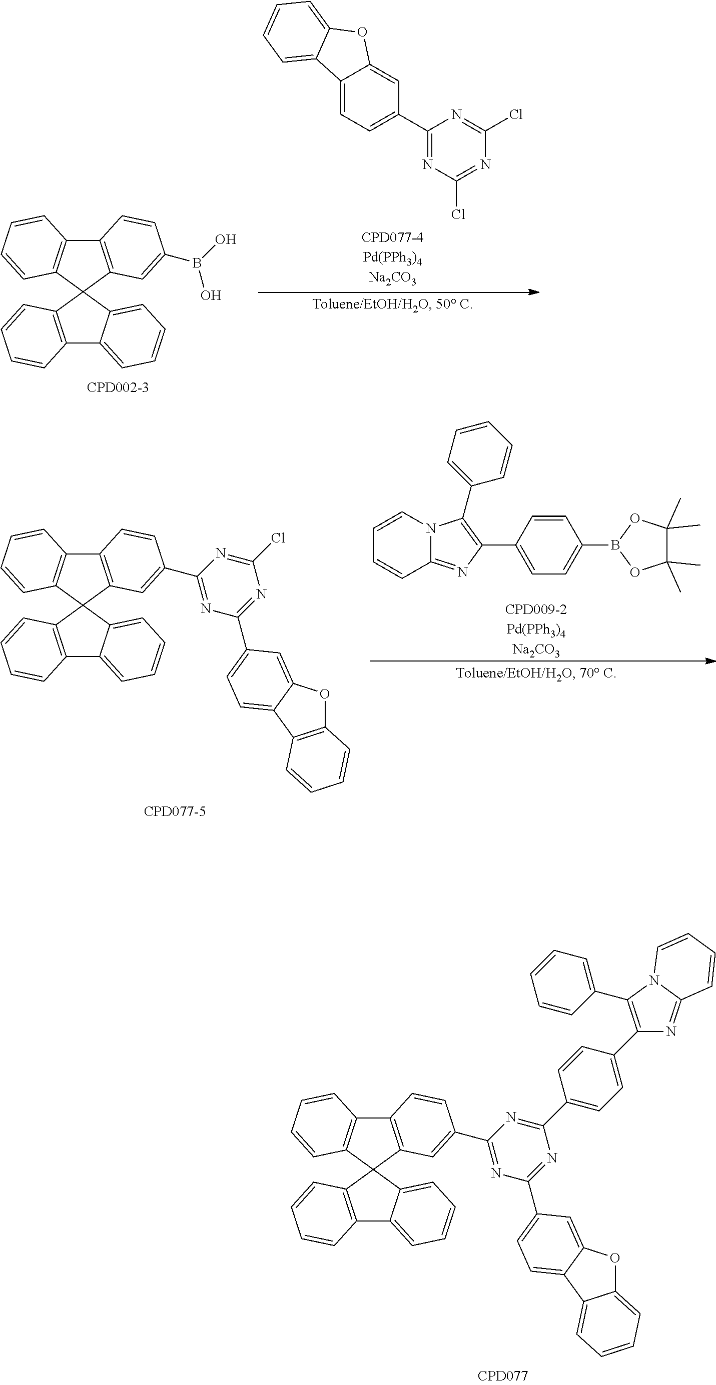 [SMILES: Clc1nc(Cl)nc(-c2ccc3c(c2)oc2ccccc23)n1 ; OB(O)c1ccc2c(c1)C1(c3ccccc3-c3ccccc31)c1ccccc1-2 ; Clc1nc(-c2ccc3c(c2)C2(c4ccccc4-c4ccccc42)c2ccccc2-3)nc(-c2ccc3c(c2)oc2ccccc23)n1 ; CC1(C)OB(c2ccc(-c3nc4ccccn4c3-c3ccccc3)cc2)OC1(C)C ; "CPD009-2Pd(PPh3)4Na2CO3" ; c1ccc(-c2c(-c3ccc(-c4nc(-c5ccc6c(c5)C5(c7ccccc7-c7ccccc75)c5ccccc5-6)nc(-c5ccc6c(c5)oc5ccccc56)n4)cc3)nc3ccccn23)cc1]

### Synthesis of a Compound CPD077-5

With reference to the synthesis and purification methods of the compound CPD041-5, only the corresponding raw materials were required to be changed, and a target compound CPD077-5 (19.57 g, purity: 99.72%, yield: 65.64%) was obtained. Mass spectrometry was performed at 596.25 (M+H).

### Synthesis of a Compound CPD077

With reference to the synthesis and purification methods of the compound CPD041, only the corresponding raw materials were required to be changed, and a target compound CPD077 (18.47 g, purity: 99.92%, yield: 70.03%) was obtained. 18.47 g of the crude product CPD077 was sublimated and purified to obtain sublimated and purified CPD077 (10.04 g, purity: 99.93%, yield: 54.35%). Mass spectrometry was performed at 830.20 (M+H).

¹H NMR (400 MHz, CDCl₃) δ 8.8-8.78 (m, 1H), 8.67-8.59 (m, 2H), 8.52 (dd, J=23.4, 2.9 Hz, 2H), 8.40 (d, J=15.0 Hz, 1H), 8.36-8.25 (m, 3H), 8.25-8.07 (m, 6H), 8.03-7.92 (m, 4H), 7.92-7.77 (m, 6H), 7.75-7.63 (m, 4H), 7.62-7.47 (m, 5H), 7.21-7.15 (m, 1H).

### Synthesis of a Compound CPD102

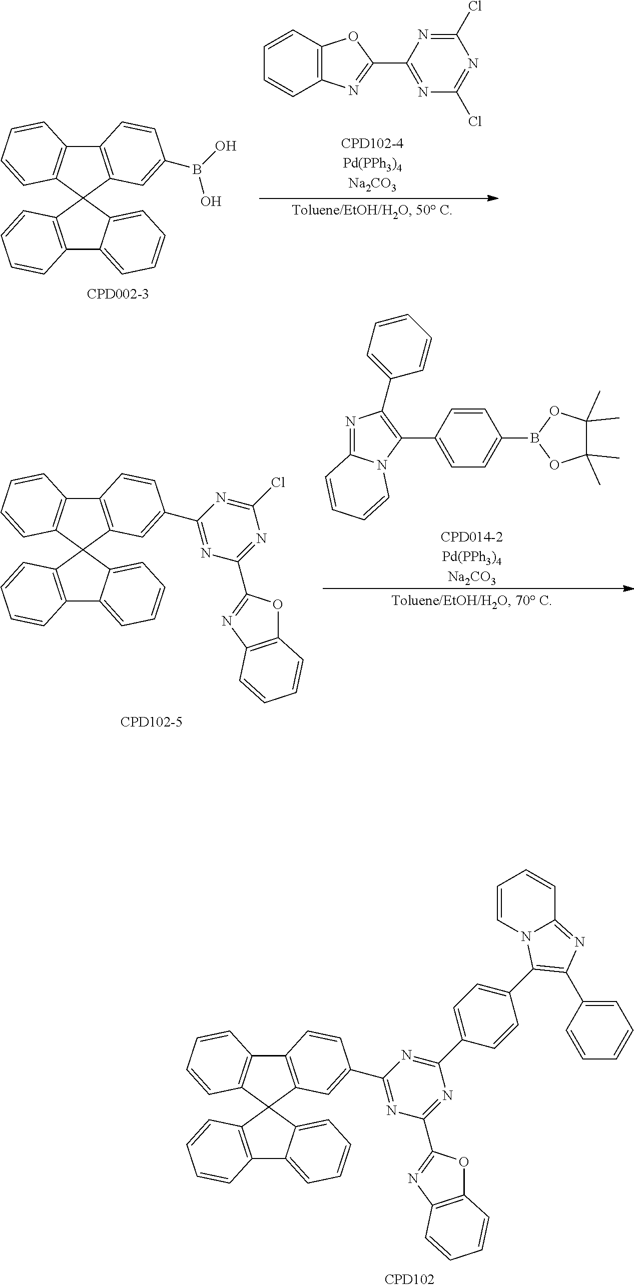 [SMILES: Clc1nc(Cl)nc(-c2nc3ccccc3o2)n1 ; OB(O)c1ccc2c(c1)C1(c3ccccc3-c3ccccc31)c1ccccc1-2 ; Clc1nc(-c2ccc3c(c2)C2(c4ccccc4-c4ccccc42)c2ccccc2-3)nc(-c2nc3ccccc3o2)n1 ; CC1(C)OB(c2ccc(-c3c(-c4ccccc4)nc4ccccn34)cc2)OC1(C)C ; "CPD014-2Pd(PPh3)4Na2CO3" ; c1ccc(-c2nc3ccccn3c2-c2ccc(-c3nc(-c4ccc5c(c4)C4(c6ccccc6-c6ccccc64)c4ccccc4-5)nc(-c4nc5ccccc5o4)n3)cc2)cc1]

### Synthesis of a Compound CPD102-5

With reference to the synthesis and purification methods of the compound CPD041-5, only the corresponding raw materials were required to be changed, and a target compound CPD102-5 (15.43 g, purity: 99.48%, yield: 64.37%) was obtained. Mass spectrometry was performed at 547.22 (M+H).

### Synthesis of a Compound CPD102

With reference to the synthesis and purification methods of the compound CPD041, only the corresponding raw materials were required to be changed, and a target compound CPD102 (12.64 g, purity: 99.96%, yield: 75.62%) was obtained. 12.64 g of the crude product CPD102 was sublimated and purified to obtain sublimated and purified CPD102 (6.42 g, purity: 99.96%, yield: 50.79%). Mass spectrometry was performed at 781.16 (M+H).

¹H NMR (400 MHz, CDCl₃) δ 8.83-8.79 (m, 1H), 8.67-8.60 (m, 2H), 8.56 (d, J=3.1 Hz, 1H), 8.41 (d, J=15.0 Hz, 1H), 8.32-8.18 (m, 5H), 8.13-7.90 (m, 6H), 7.89-7.76 (m, 4H), 7.76-7.48 (m, 11H), 7.22-7.16 (m, 1H).

### Synthesis of a Compound CPD111

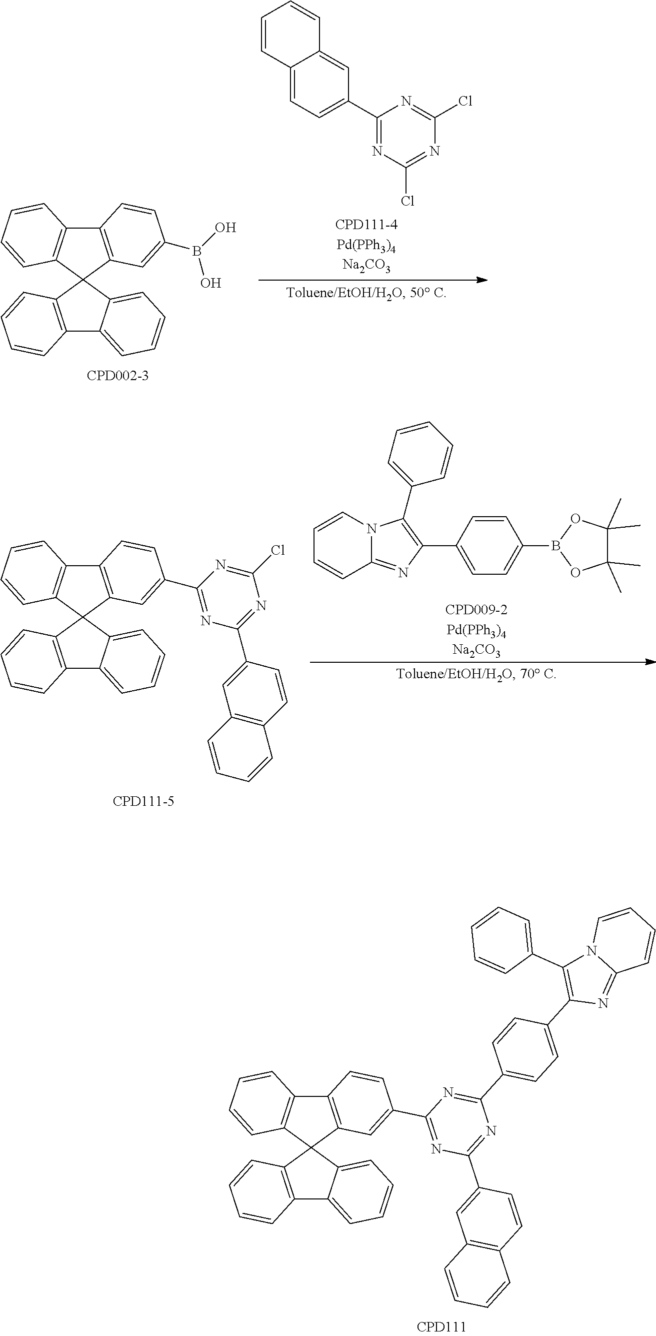

### Synthesis of a Compound CPD111-5

With reference to the synthesis and purification methods of the compound CPD041-5, only the corresponding raw materials were required to be changed, and a target compound CPD111-5 (25.19 g, purity: 99.60%, yield: 62.37%) was obtained. Mass spectrometry was performed at 556.15 (M+H).

### Synthesis of a Compound CPD111

With reference to the synthesis and purification methods of the compound CPD041, only the corresponding raw materials were required to be changed, and a target compound CPD111 (30.61 g, purity: 99.95%, yield: 72.84%) was obtained. 30.61 g of the crude product CPD111 was sublimated and purified to obtain sublimated and purified CPD111 (18.93 g, purity: 99.95%, yield: 61.84%). Mass spectrometry was performed at 790.22 (M+H).

¹H NMR (400 MHz, CDCl₃) δ 8.81-8.79 (m, 1H), 8.67-8.59 (m, 2H), 8.55-8.32 (m, 3H), 8.21-8.00 (m, 2H), 7.94-7.84 (m, 1H), 7.77-7.51 (m, 8H), 7.48-7.18 (m, 10H), 7.07 (td, J=14.9, 3.4 Hz, 2H), 7.02-6.88 (m, 5H), 7.23-7.15 (m, 1H).

### Manufacturing of Organic Electroluminescent Devices

A glass substrate with a size of 50 mm*50 mm*1.0 mm including an ITO (100 nm) transparent electrode was ultrasonically cleaned in ethanol for 10 min, dried at 150° C., and then treated with N₂ plasma for 30 min. The washed glass substrate was installed on a substrate support of a vacuum evaporation device. At first, a compound HATCN for covering the transparent electrode was evaporated on the surface of the side having a transparent electrode line to form a thin film with a thickness of 5 nm. Then, a layer of HTM1 was evaporated to form a thin film with a thickness of 60 nm. Then, a layer of HTM2 was evaporated on the HTM1 thin film to form a thin film with a thickness of 10 nm. Then, a main material CBP and a doping material were co-evaporated on the HTM2 film layer to obtain a film with a thickness of 30 nm, where the ratio of the main material to the doping material was 90%:10%. HBL (5 nm) and ETL (30 nm) were sequentially evaporated on a light-emitting layer to serve as a hole blocking layer material and an electron transport material respectively according to combinations in the following table. Then, LiF (1 nm) was evaporated on the electron transport material layer to serve as an electron injection material. Then, a mixture of Mg and Ag (18 nm, at a ratio of 1:9) was co-evaporated to serve as a cathode material.

[symbol]

### Evaluation of Properties of Devices

Properties of devices obtained above were tested. In various examples and comparative examples, a constant-current power supply (Keithley 2400) was used, a current at a fixed density was used for flowing through light-emitting elements, and a spectroradiometer (CS 2000) was used for testing the light-emitting spectrum. Meanwhile, the voltage value was measured, and the time (LT90) when the brightness was reduced to 90% of the initial brightness was tested. Results are shown in the following Table 2.

TABLE 2

| | | Starting | Current | LT90 |
|---|---|---|---|---|
| | | voltage V | efficiency Cd/A | @ |
| | ETL | @ 3000 nits | @ 3000 nits | 3000 nits |
| Example 1 | CPD002 | 4.4 | 64 | 176 |
| Example 2 | CPD009 | 4.3 | 63 | 189 |
| Example 3 | CPD014 | 4.6 | 62 | 186 |
| Example 4 | CPD017 | 4.3 | 67 | 196 |
| Example 5 | CPD026 | 4.1 | 69 | 192 |
| Example 6 | CPD027 | 4.4 | 67 | 183 |
| Example 7 | CPD033 | 4.3 | 66 | 174 |
| Example 8 | CPD041 | 4.6 | 71 | 207 |
| Example 9 | CPD046 | 4.4 | 70 | 201 |
| Example 10 | CPD054 | 4.3 | 66 | 154 |
| Example 11 | CPD059 | 4.5 | 68 | 161 |
| Example 12 | CPD065 | 4.4 | 69 | 203 |
| Example 13 | CPD072 | 4.7 | 72 | 217 |
| Example 14 | CPD074 | 4.6 | 70 | 231 |
| Example 15 | CPD075 | 4.6 | 68 | 249 |
| Example 16 | CPD077 | 4.5 | 63 | 233 |
| Example 17 | CPD102 | 4.6 | 62 | 118 |
| Example 18 | CPD111 | 4.4 | 65 | 169 |
| Comparative | Reference | 4.8 | 47 | 86 |
| Example 1 | compound 1 | | | |
| Comparative | Reference | 4.9 | 53 | 127 |
| Example 2 | compound 2 | | | |
| Comparative | Reference | 5.1 | 51 | 106 |
| Example 3 | compound 3 | | | |

Comparison of the sublimation temperature is as follows. The sublimation temperature is defined as the temperature corresponding to an evaporation rate of 1 Å/s at a vacuum degree of 10⁻⁷ Torr. Test results are shown as follows.

| Material | Sublimation temperature |
|---|---|
| CPD002 | 269 |
| CPD026 | 266 |
| CPD046 | 274 |
| CPD072 | 276 |
| CPD075 | 276 |
| Reference compound 1 | 261 |
| Reference compound 2 | 287 |
| Reference compound 3 | 279 |

Through comparison of the data in the above table, it can be seen that compared with reference compounds 1-3, the compound of the present invention used as an electron transport layer in an organic electroluminescent device has more excellent properties, such as driving voltage, luminous efficiency and device service life.

The above results show that the compound of the present invention has the advantages of low sublimation temperature, good optical, electrical and thermal stability, a high refractive index, small differences of the refractive index in a visible light region and the like. A device prepared from the compound has the advantages of low voltage, long service life, high luminous efficiency and the like. The compound can be used in organic light-emitting devices. In particular, the compound has the possibility of being applied to the AMOLED industry as an electron transport material.

By connecting N heterocyclic ring with imidazopyrimidine ring on spirofluorene, the material of the present invention has the advantages of high optical and electrical stability, low sublimation temperature, low driving voltage, high luminous efficiency, long device service life and the like, and can be used in an organic electroluminescent device. In particular. the compound has the possibility of being applied in the AMOLED industry as an electron transport material.

1. A spiro compound, having a structure as shown in a formula (1), 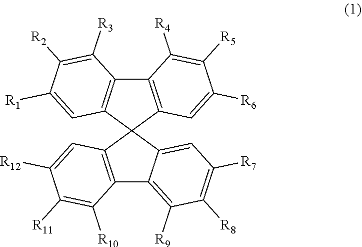
wherein R₁-R₁₂ are independently selected from hydrogen, deuterium, halogen, cyano, hydroxyl, sulfhydryl, amino, substituted or unsubstituted C₁-C₁₀ alkyl, substituted or unsubstituted C₁-C₁₀heteroalkyl, substituted or unsubstituted C₃-C₂₀ cycloalkyl, substituted or unsubstituted C₃-C₂₀heterocycloalkyl, substituted or unsubstituted C₂-C₁₀ alkenyl, substituted or unsubstituted C₂-C₁₀ alkynyl, substituted or unsubstituted C₆-C₃₀ aryl, substituted or unsubstituted C₂-C₃₀ heteroaryl, substituted or unsubstituted tri-C₁-C₁₀alkylsilyl, substituted or unsubstituted tri-C₆-C₁₂arylsilyl, substituted or unsubstituted di-C₁-C₁₀ alkyl mono-C₆-C₃₀arylsilyl, and substituted or unsubstituted mono-C₁-C₁₀ alkyl di-C₆-C₃₀arylsilyl, or two adjacent groups are connected to each other to form an aliphatic ring, a heteroaromatic ring or an aromatic ring structure; and
at least one of the R₁-R₁₂ has a structure as shown in a formula (2), 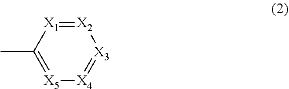
wherein in the formula (2),
X₁-X₅ are independently selected from N or CR₀; in the X₁-X_{5,} at least one is N, and at least one is CR₀;
R₀ is independently selected from hydrogen, deuterium, halogen, cyano, hydroxyl, sulfhydryl, amino, substituted or unsubstituted C₁-C₁₀ alkyl, substituted or unsubstituted C₁-C₁₀heteroalkyl, substituted or unsubstituted C₃-C₂₀ cycloalkyl, substituted or unsubstituted C₃-C₂₀heterocycloalkyl, substituted or unsubstituted C₂-C₁₀ alkenyl, substituted or unsubstituted C₂-C₁₀ alkynyl, substituted or unsubstituted C₆-C₃₀ aryl, substituted or unsubstituted C₂-C₃₀ heteroaryl, substituted or unsubstituted tri-C₁-C₁₀alkylsilyl, substituted or unsubstituted tri-C₆-C₁₂arylsilyl, substituted or unsubstituted di-C₁-C₁₀ alkyl mono-C₆-C₃₀arylsilyl, and substituted or unsubstituted mono-C₁-C₁₀ alkyl di-C₆-C₃₀arylsilyl, or two adjacent groups are connected to each other to form an aliphatic ring, a heteroaromatic ring or an aromatic ring structure; and
at least one of the R₀ has a structure as shown in a formula (3) or a formula (4), 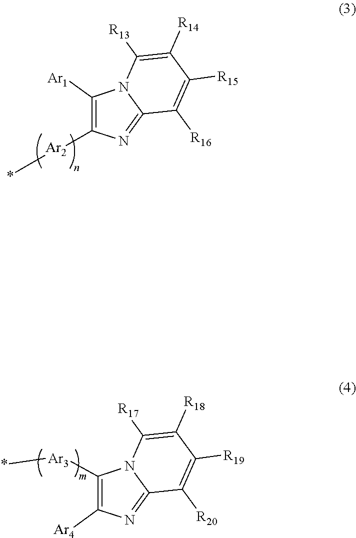
wherein * indicates a position connected with the formula (2);
m and n are independently selected from an integer of 0-4, and when the n or the m is 0, the formula (3) or the formula (4) is directly connected with the formula (2);
R₁₃-R₂₀ are independently selected from hydrogen, deuterium, halogen, cyano, hydroxyl, sulfhydryl, amino, substituted or unsubstituted C₁-C₁₀ alkyl, substituted or unsubstituted C₁-C₁₀heteroalkyl, substituted or unsubstituted C₃-C₂₀ cycloalkyl, substituted or unsubstituted C₃-C₂₀heterocycloalkyl, substituted or unsubstituted C₂-C₁₀ alkenyl, substituted or unsubstituted C₂-C₁₀ alkynyl, substituted or unsubstituted C₆-C₃₀ aryl, substituted or unsubstituted C₂-C₃₀ heteroaryl, substituted or unsubstituted tri-C₁-C₁₀alkylsilyl, substituted or unsubstituted tri-C₆-C₁₂arylsilyl, substituted or unsubstituted di-C₁-C₁₀ alkyl mono-C₆-C₃₀arylsilyl, and substituted or unsubstituted mono-C₁-C₁₀ alkyl di-C₆-C₃₀arylsilyl, or two adjacent groups are connected to each other to form an aliphatic ring, a heteroaromatic ring or an aromatic ring structure;
Ar₁-Ar₄ are independently selected from substituted or unsubstituted C₆-C₃₀ aryl, or substituted or unsubstituted C₄-C₃₀ heteroaryl;
the heteroalkyl, the heterocycloalkyl, the heteroaromatic ring and the heteroaryl at least contain one O, N, or S heteroatom;
and the “substituted” refers to substitution with deuterium, F, Cl, Br, C₁-C₆ alkyl, C₃-C₆ cycloalkyl, C₁-C₆ alkylamino, C₆-C₁₀ aryl, cyano, isocyano, or phosphino, and the substitution ranges from a single substitution number to a maximum substitution number.

2. The spiro compound according to claim 1, having a structure as shown in a formula (5) or a formula (6), 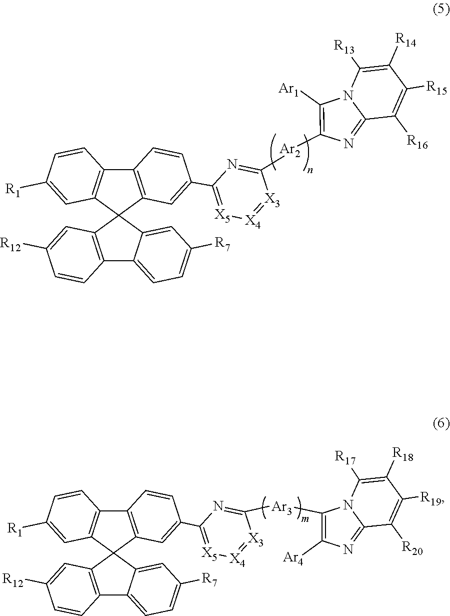
wherein R₁, R₇, R₁₂ and R₁₃-R₂₀ are independently selected from hydrogen, deuterium, halogen, cyano, hydroxyl, sulfhydryl, amino, substituted or unsubstituted C₁-C₁₀ alkyl, substituted or unsubstituted C₁-C₁₀heteroalkyl, substituted or unsubstituted C₃-C₂₀ cycloalkyl, substituted or unsubstituted C₃-C₂₀heterocycloalkyl, substituted or unsubstituted C₂-C₁₀ alkenyl, substituted or unsubstituted C₂-C₁₀ alkynyl, substituted or unsubstituted C₆-C₃₀ aryl, substituted or unsubstituted C₂-C₃₀ heteroaryl, substituted or unsubstituted tri-C₁-C₁₀alkylsilyl, substituted or unsubstituted tri-C₆-C₁₂arylsilyl, substituted or unsubstituted di-C₁-C₁₀ alkyl mono-C₆-C₃₀arylsilyl, and substituted or unsubstituted mono-C₁-C₁₀ alkyl di-C₆-C₃₀arylsilyl, or two adjacent groups are connected to each other to form an aliphatic ring, a heteroaromatic ring or an aromatic ring structure;
X₃-X₅ are independently selected from N or CR₀;
R₀ is independently selected from hydrogen, deuterium, halogen, cyano, hydroxyl, sulfhydryl, amino, substituted or unsubstituted C₁-C₁₀ alkyl, substituted or unsubstituted C₁-C₁₀heteroalkyl, substituted or unsubstituted C₃-C₂₀ cycloalkyl, substituted or unsubstituted C₃-C₂₀heterocycloalkyl, substituted or unsubstituted C₂-C₁₀ alkenyl, substituted or unsubstituted C₂-C₁₀ alkynyl, substituted or unsubstituted C₆-C₃₀ aryl, substituted or unsubstituted C₂-C₃₀ heteroaryl, substituted or unsubstituted tri-C₁-C₁₀alkylsilyl, substituted or unsubstituted tri-C₆-C₁₂arylsilyl, substituted or unsubstituted di-C₁-C₁₀ alkyl mono-C₆-C₃₀arylsilyl, and substituted or unsubstituted mono-C₁-C₁₀ alkyl di-C₆-C₃₀arylsilyl, or two adjacent groups are connected to each other to form an aliphatic ring, a heteroaromatic ring or an aromatic ring structure;
m and n are independently selected from an integer of 0-4;
Ar₁-Ar₄ are independently selected from substituted or unsubstituted C₆-C₃₀ aryl, or substituted or unsubstituted C₄-C₃₀ heteroaryl;
the heteroalkyl, the heterocycloalkyl, the heteroaromatic ring and the heteroaryl at least contain one O, N, or S heteroatom;
and the “substituted” refers to substitution with deuterium, F, Cl, Br, C₁-C₆ alkyl, C₃-C₆ cycloalkyl, C₁-C₆ alkylamino, C₆-C₁₀ aryl, cyano, isocyano, or phosphino, and the substitution ranges from a single substitution number to a maximum substitution number.

3. The spiro compound according to claim 2, wherein the R₁, the R₇, the R₁₂ and the R₁₃-R₂₀ are independently selected from hydrogen, deuterium, halogen, cyano, substituted or unsubstituted C₁-C₁₀ alkyl, substituted or unsubstituted C₃-C₂₀ cycloalkyl, substituted or unsubstituted C₆-C₃₀ aryl, and substituted or unsubstituted C₂-C₃₀ heteroaryl, or two adjacent groups are connected to each other to form a heteroaromatic ring or an aromatic ring structure;
the X₃-X₅ are independently selected from N or CR₀;
the R₀ is independently selected from hydrogen, deuterium, halogen, cyano, substituted or unsubstituted C₁-C₁₀ alkyl, substituted or unsubstituted C₃-C₂₀ cycloalkyl, substituted or unsubstituted C₆-C₃₀ aryl, and substituted or unsubstituted C₂-C₃₀ heteroaryl, or two adjacent groups are connected to each other to form a heteroaromatic ring or an aromatic ring structure;
the m and the n are independently selected from an integer of 0-2;
the Ar₁-Ar₄ are independently selected from substituted or unsubstituted C₆-C₃₀ aryl, or substituted or unsubstituted C₄-C₃₀ heteroaryl;
the heteroaromatic ring and the heteroaryl at least contain one O or N heteroatom;
and the “substituted” refers to substitution with deuterium, F, Cl, Br, C₁-C₆ alkyl, C₃-C₆ cycloalkyl, or C₆-C₁₀ aryl, and the substitution ranges from a single substitution number to a maximum substitution number.

4. The spiro compound according to claim 3, wherein in the X₃-X₅, at least one is N.

5. The spiro compound according to claim 4, wherein in the X₃-X₅, the X₃ and/or the X₅ is N.

6. The spiro compound according to claim 5, wherein when the X₃ and/or the X₅ is N, the X₄ is CR₀, and the R₀ is not H.

7. The spiro compound according to claim 6, wherein when the X₃ and the X₅ are N, the X₄ is CR₀, and the R₀ is substituted or unsubstituted C₆-C₃₀ aryl, or substituted or unsubstituted C₂-C₃₀ heteroaryl.

8. The spiro compound according to claim 4, wherein when the X₅ is N and the X₄ and the X₃ are CR₀, one of two R₀ is substituted or unsubstituted C₆-C₂₀ aryl, or substituted or unsubstituted C₂-C₂₀ heteroaryl, and the other one is hydrogen, or the two R₀ are connected to each other to form an aromatic ring or a heteroaromatic ring; and when the X₃ is N and the X₄ and the X₅ are CR₀, one of two R₀ is substituted or unsubstituted C₆-C₂₀ aryl, or substituted or unsubstituted C₂-C₂₀ heteroaryl, and the other one is hydrogen, or the two R₀ are connected to each other to form an aromatic ring or a heteroaromatic ring.

9. The spiro compound according to any one of claims 1, wherein the m or the n is an integer of 1-2.

10. The spiro compound according to claim 9, wherein the Ar₁-Ar₄ are independently selected from substituted or unsubstituted C₆-C₁₈ aromatic ring, or substituted or unsubstituted C₄-C₁₈ heteroaryl.

11. The spiro compound according to claim 10, wherein the Ar₁-Ar₄ are independently selected from substituted or unsubstituted C₆-C₁₀ aromatic ring.

12. The spiro compound according to claim 10, wherein at least one of the R₁, the R₇ and the R₁₂ is F or CN, and the other groups are hydrogen.

13. The spiro compound according to claim 10, wherein the R₁, the R₇ and the R₁₂ are hydrogen.

14. The spiro compound according to claim 13, wherein at least one group of two adjacent substituents in the R₁₃-R₁₆ and the R₁₇-R₂₀ are connected to each other to form an aromatic ring or a heteroaromatic ring structure, and the other groups are hydrogen.

15. The spiro compound according to claim 13, wherein the R₁₃-R₁₆ and the R₁₇-R₂₀ are hydrogen.

16. The spiro compound according to claim 2, wherein the spiro compound has one of the following structural formulas, or is partially or completely deuterated or fluorinated correspondingly.

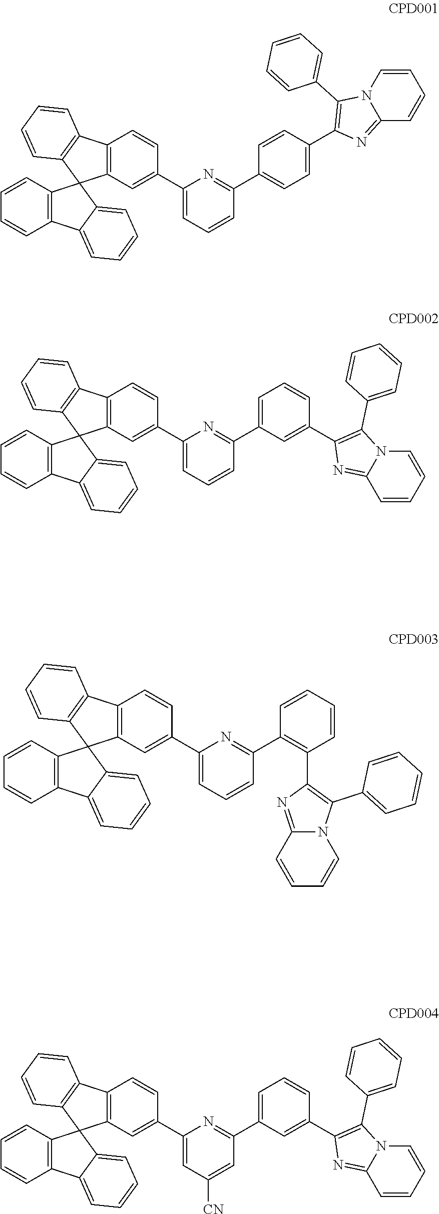 [SMILES: c1ccc(-c2c(-c3ccc(-c4cccc(-c5ccc6c(c5)C5(c7ccccc7-c7ccccc75)c5ccccc5-6)n4)cc3)nc3ccccn23)cc1 ; c1ccc(-c2c(-c3cccc(-c4cccc(-c5ccc6c(c5)C5(c7ccccc7-c7ccccc75)c5ccccc5-6)n4)c3)nc3ccccn23)cc1 ; c1ccc(-c2c(-c3ccccc3-c3cccc(-c4ccc5c(c4)C4(c6ccccc6-c6ccccc64)c4ccccc4-5)n3)nc3ccccn23)cc1 ; N#Cc1cc(-c2cccc(-c3nc4ccccn4c3-c3ccccc3)c2)nc(-c2ccc3c(c2)C2(c4ccccc4-c4ccccc42)c2ccccc2-3)c1]

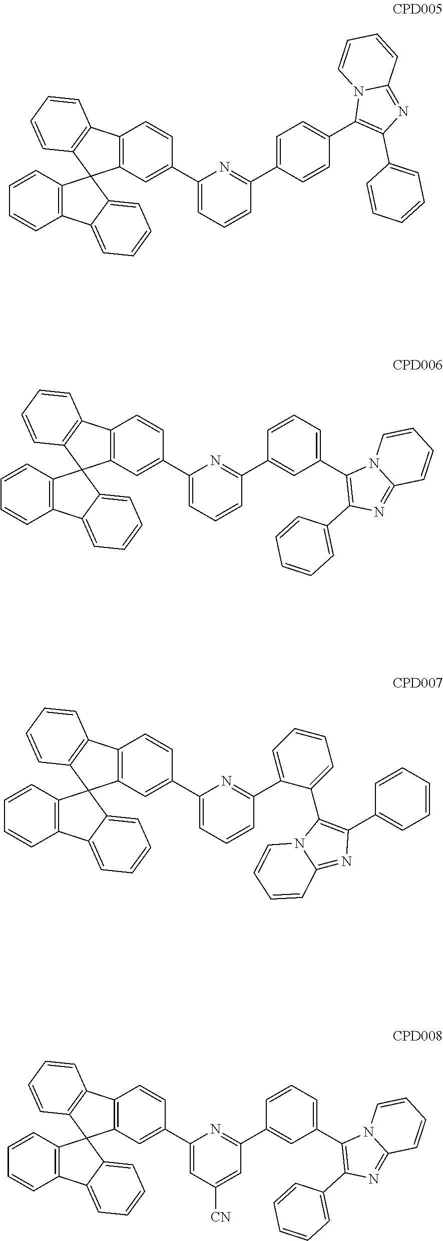 [SMILES: c1ccc(-c2nc3ccccn3c2-c2ccc(-c3cccc(-c4ccc5c(c4)C4(c6ccccc6-c6ccccc64)c4ccccc4-5)n3)cc2)cc1 ; c1ccc(-c2nc3ccccn3c2-c2cccc(-c3cccc(-c4ccc5c(c4)C4(c6ccccc6-c6ccccc64)c4ccccc4-5)n3)c2)cc1 ; c1ccc(-c2nc3ccccn3c2-c2ccccc2-c2cccc(-c3ccc4c(c3)C3(c5ccccc5-c5ccccc53)c3ccccc3-4)n2)cc1 ; N#Cc1cc(-c2cccc(-c3c(-c4ccccc4)nc4ccccn34)c2)nc(-c2ccc3c(c2)C2(c4ccccc4-c4ccccc42)c2ccccc2-3)c1]

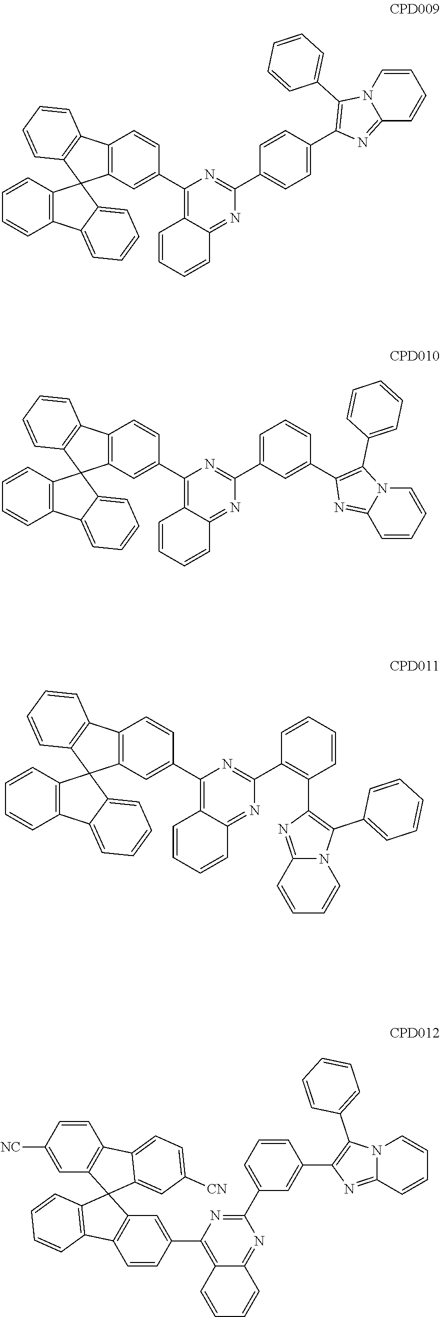 [SMILES: c1ccc(-c2c(-c3ccc(-c4nc(-c5ccc6c(c5)C5(c7ccccc7-c7ccccc75)c5ccccc5-6)c5ccccc5n4)cc3)nc3ccccn23)cc1 ; c1ccc(-c2c(-c3cccc(-c4nc(-c5ccc6c(c5)C5(c7ccccc7-c7ccccc75)c5ccccc5-6)c5ccccc5n4)c3)nc3ccccn23)cc1 ; c1ccc(-c2c(-c3ccccc3-c3nc(-c4ccc5c(c4)C4(c6ccccc6-c6ccccc64)c4ccccc4-5)c4ccccc4n3)nc3ccccn23)cc1 ; N#Cc1ccc2c(c1)C1(c3ccccc3-c3ccc(-c4nc(-c5cccc(-c6nc7ccccn7c6-c6ccccc6)c5)nc5ccccc45)cc31)c1cc(C#N)ccc1-2]

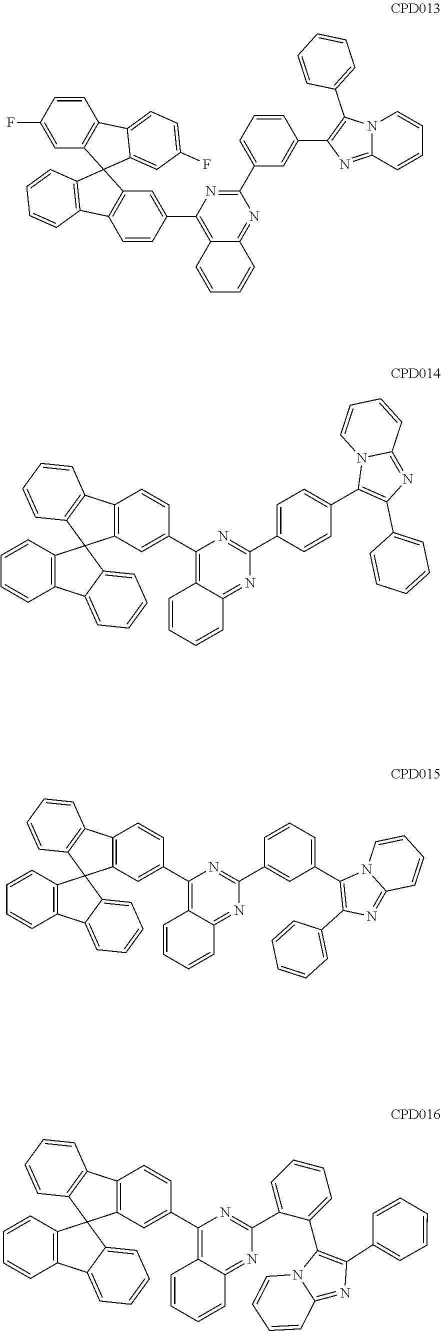 [SMILES: Fc1ccc2c(c1)C1(c3ccccc3-c3ccc(-c4nc(-c5cccc(-c6nc7ccccn7c6-c6ccccc6)c5)nc5ccccc45)cc31)c1cc(F)ccc1-2 ; c1ccc(-c2nc3ccccn3c2-c2ccc(-c3nc(-c4ccc5c(c4)C4(c6ccccc6-c6ccccc64)c4ccccc4-5)c4ccccc4n3)cc2)cc1 ; c1ccc(-c2nc3ccccn3c2-c2cccc(-c3nc(-c4ccc5c(c4)C4(c6ccccc6-c6ccccc64)c4ccccc4-5)c4ccccc4n3)c2)cc1 ; c1ccc(-c2nc3ccccn3c2-c2ccccc2-c2nc(-c3ccc4c(c3)C3(c5ccccc5-c5ccccc53)c3ccccc3-4)c3ccccc3n2)cc1]

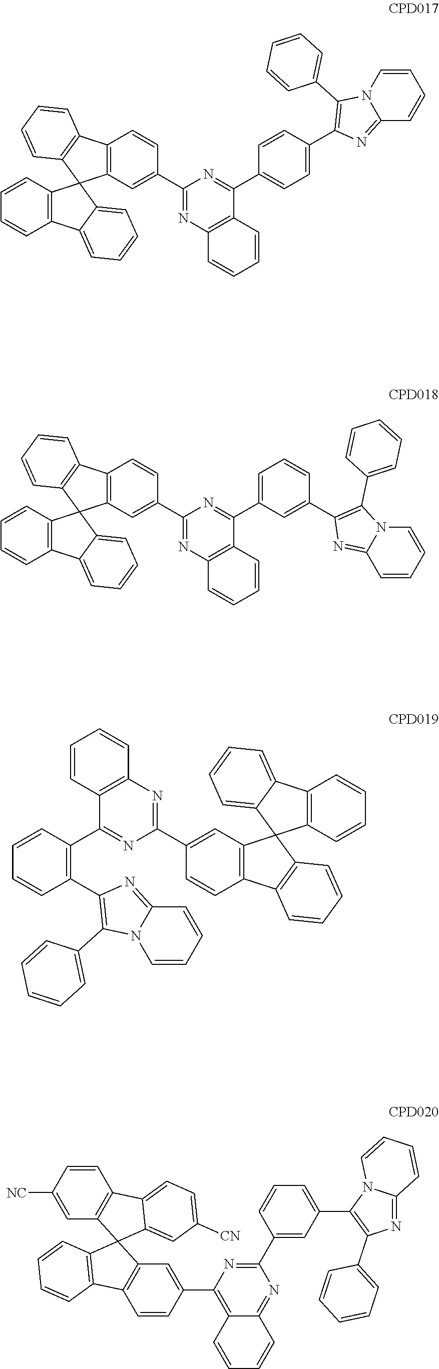 [SMILES: c1ccc(-c2c(-c3ccc(-c4nc(-c5ccc6c(c5)C5(c7ccccc7-c7ccccc75)c5ccccc5-6)nc5ccccc45)cc3)nc3ccccn23)cc1 ; c1ccc(-c2c(-c3cccc(-c4nc(-c5ccc6c(c5)C5(c7ccccc7-c7ccccc75)c5ccccc5-6)nc5ccccc45)c3)nc3ccccn23)cc1 ; c1ccc(-c2c(-c3ccccc3-c3nc(-c4ccc5c(c4)C4(c6ccccc6-c6ccccc64)c4ccccc4-5)nc4ccccc34)nc3ccccn23)cc1 ; N#Cc1ccc2c(c1)C1(c3ccccc3-c3ccc(-c4nc(-c5cccc(-c6c(-c7ccccc7)nc7ccccn67)c5)nc5ccccc45)cc31)c1cc(C#N)ccc1-2]

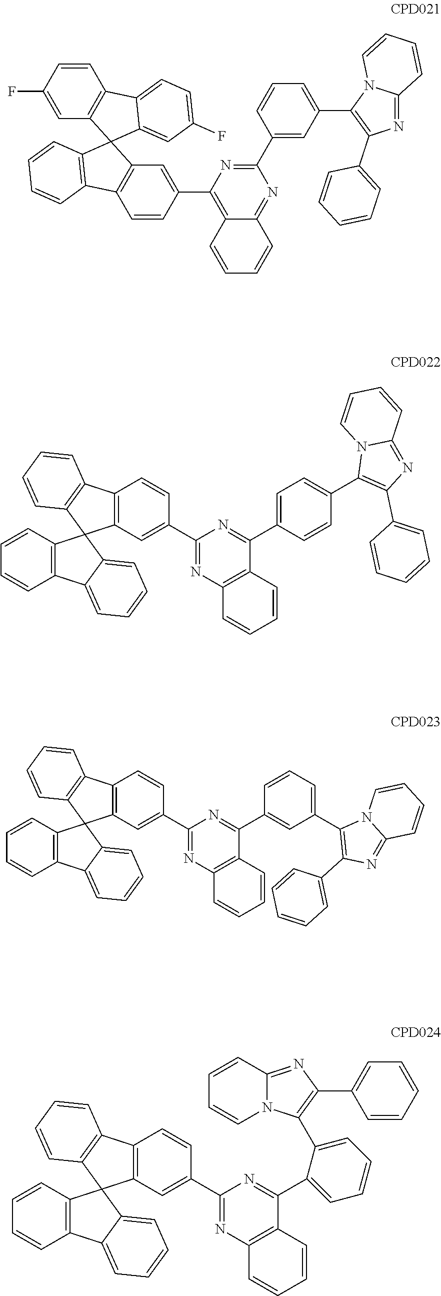 [SMILES: Fc1ccc2c(c1)C1(c3ccccc3-c3ccc(-c4nc(-c5cccc(-c6c(-c7ccccc7)nc7ccccn67)c5)nc5ccccc45)cc31)c1cc(F)ccc1-2 ; c1ccc(-c2nc3ccccn3c2-c2ccc(-c3nc(-c4ccc5c(c4)C4(c6ccccc6-c6ccccc64)c4ccccc4-5)nc4ccccc34)cc2)cc1 ; c1ccc(-c2nc3ccccn3c2-c2cccc(-c3nc(-c4ccc5c(c4)C4(c6ccccc6-c6ccccc64)c4ccccc4-5)nc4ccccc34)c2)cc1 ; c1ccc(-c2nc3ccccn3c2-c2ccccc2-c2nc(-c3ccc4c(c3)C3(c5ccccc5-c5ccccc53)c3ccccc3-4)nc3ccccc23)cc1]

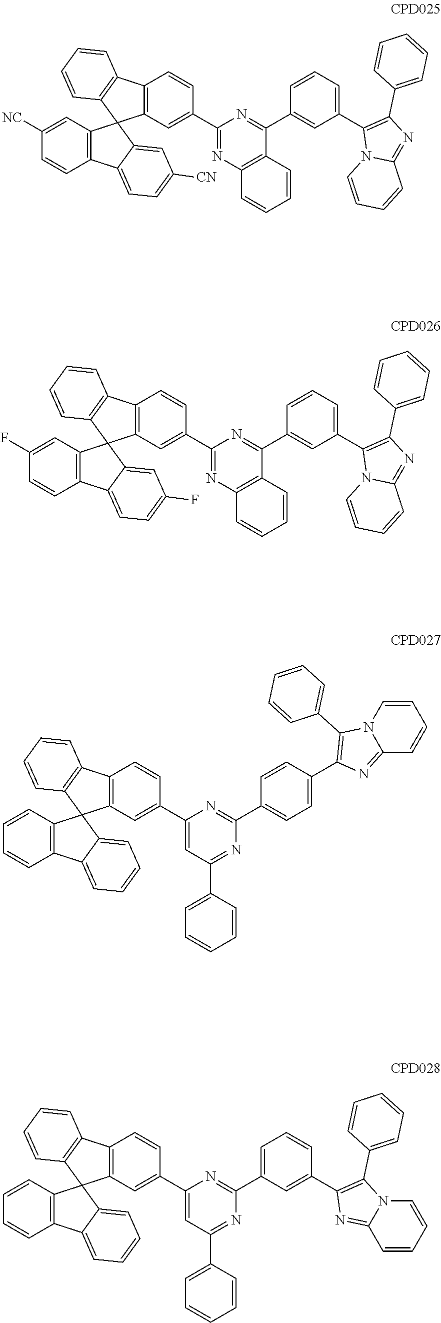 [SMILES: N#Cc1ccc2c(c1)C1(c3ccccc3-c3ccc(-c4nc(-c5cccc(-c6c(-c7ccccc7)nc7ccccn67)c5)c5ccccc5n4)cc31)c1cc(C#N)ccc1-2 ; Fc1ccc2c(c1)C1(c3ccccc3-c3ccc(-c4nc(-c5cccc(-c6c(-c7ccccc7)nc7ccccn67)c5)c5ccccc5n4)cc31)c1cc(F)ccc1-2 ; c1ccc(-c2cc(-c3ccc4c(c3)C3(c5ccccc5-c5ccccc53)c3ccccc3-4)nc(-c3ccc(-c4nc5ccccn5c4-c4ccccc4)cc3)n2)cc1 ; c1ccc(-c2cc(-c3ccc4c(c3)C3(c5ccccc5-c5ccccc53)c3ccccc3-4)nc(-c3cccc(-c4nc5ccccn5c4-c4ccccc4)c3)n2)cc1]

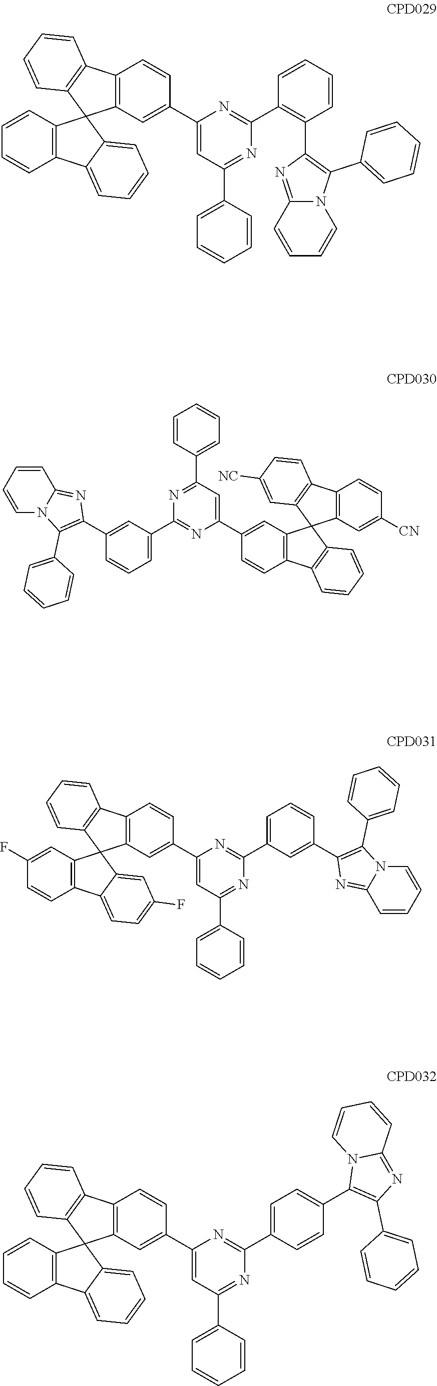 [SMILES: c1ccc(-c2cc(-c3ccc4c(c3)C3(c5ccccc5-c5ccccc53)c3ccccc3-4)nc(-c3ccccc3-c3nc4ccccn4c3-c3ccccc3)n2)cc1 ; N#Cc1ccc2c(c1)C1(c3ccccc3-c3ccc(-c4cc(-c5ccccc5)nc(-c5cccc(-c6nc7ccccn7c6-c6ccccc6)c5)n4)cc31)c1cc(C#N)ccc1-2 ; Fc1ccc2c(c1)C1(c3ccccc3-c3ccc(-c4cc(-c5ccccc5)nc(-c5cccc(-c6nc7ccccn7c6-c6ccccc6)c5)n4)cc31)c1cc(F)ccc1-2 ; c1ccc(-c2cc(-c3ccc4c(c3)C3(c5ccccc5-c5ccccc53)c3ccccc3-4)nc(-c3ccc(-c4c(-c5ccccc5)nc5ccccn45)cc3)n2)cc1]

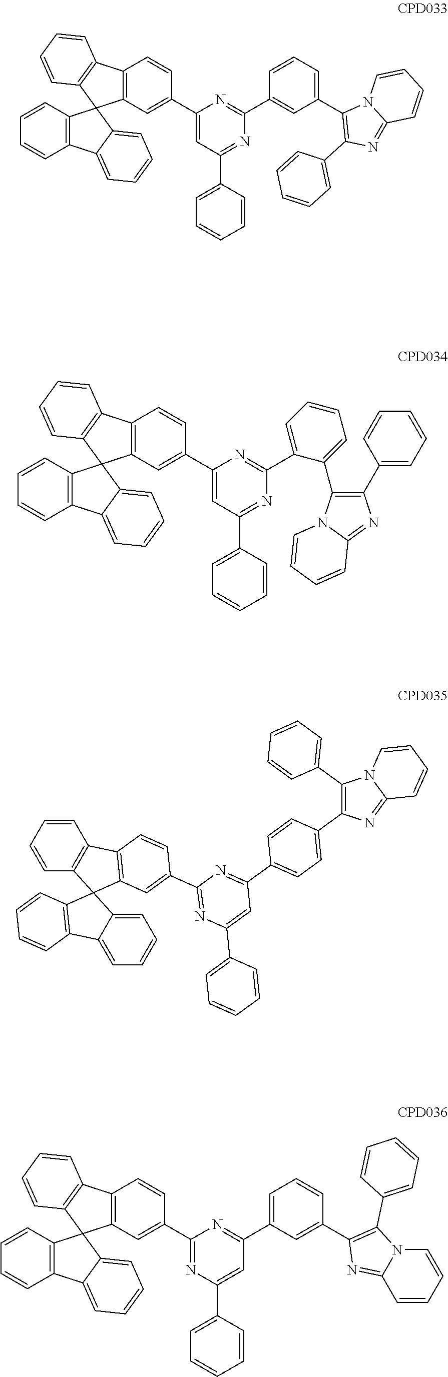 [SMILES: c1ccc(-c2cc(-c3ccc4c(c3)C3(c5ccccc5-c5ccccc53)c3ccccc3-4)nc(-c3cccc(-c4c(-c5ccccc5)nc5ccccn45)c3)n2)cc1 ; c1ccc(-c2cc(-c3ccc4c(c3)C3(c5ccccc5-c5ccccc53)c3ccccc3-4)nc(-c3ccccc3-c3c(-c4ccccc4)nc4ccccn34)n2)cc1 ; c1ccc(-c2cc(-c3ccc(-c4nc5ccccn5c4-c4ccccc4)cc3)nc(-c3ccc4c(c3)C3(c5ccccc5-c5ccccc53)c3ccccc3-4)n2)cc1 ; c1ccc(-c2cc(-c3cccc(-c4nc5ccccn5c4-c4ccccc4)c3)nc(-c3ccc4c(c3)C3(c5ccccc5-c5ccccc53)c3ccccc3-4)n2)cc1]

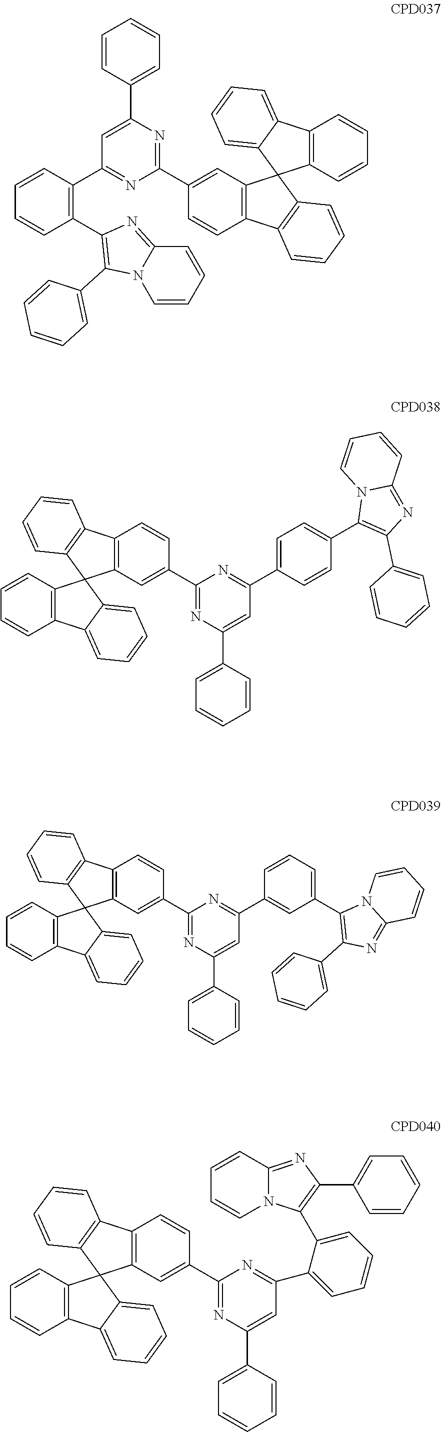 [SMILES: c1ccc(-c2cc(-c3ccccc3-c3nc4ccccn4c3-c3ccccc3)nc(-c3ccc4c(c3)C3(c5ccccc5-c5ccccc53)c3ccccc3-4)n2)cc1 ; c1ccc(-c2cc(-c3ccc(-c4c(-c5ccccc5)nc5ccccn45)cc3)nc(-c3ccc4c(c3)C3(c5ccccc5-c5ccccc53)c3ccccc3-4)n2)cc1 ; c1ccc(-c2cc(-c3cccc(-c4c(-c5ccccc5)nc5ccccn45)c3)nc(-c3ccc4c(c3)C3(c5ccccc5-c5ccccc53)c3ccccc3-4)n2)cc1 ; c1ccc(-c2cc(-c3ccccc3-c3c(-c4ccccc4)nc4ccccn34)nc(-c3ccc4c(c3)C3(c5ccccc5-c5ccccc53)c3ccccc3-4)n2)cc1]

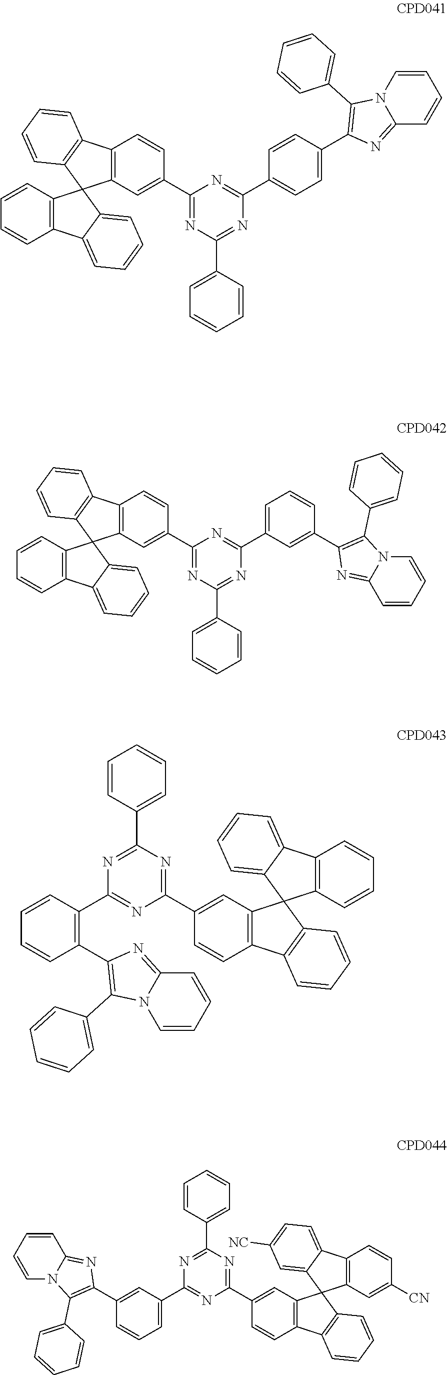 [SMILES: c1ccc(-c2nc(-c3ccc(-c4nc5ccccn5c4-c4ccccc4)cc3)nc(-c3ccc4c(c3)C3(c5ccccc5-c5ccccc53)c3ccccc3-4)n2)cc1 ; c1ccc(-c2nc(-c3cccc(-c4nc5ccccn5c4-c4ccccc4)c3)nc(-c3ccc4c(c3)C3(c5ccccc5-c5ccccc53)c3ccccc3-4)n2)cc1 ; c1ccc(-c2nc(-c3ccc4c(c3)C3(c5ccccc5-c5ccccc53)c3ccccc3-4)nc(-c3ccccc3-c3nc4ccccn4c3-c3ccccc3)n2)cc1 ; N#Cc1ccc2c(c1)C1(c3ccccc3-c3ccc(-c4nc(-c5ccccc5)nc(-c5cccc(-c6nc7ccccn7c6-c6ccccc6)c5)n4)cc31)c1cc(C#N)ccc1-2]

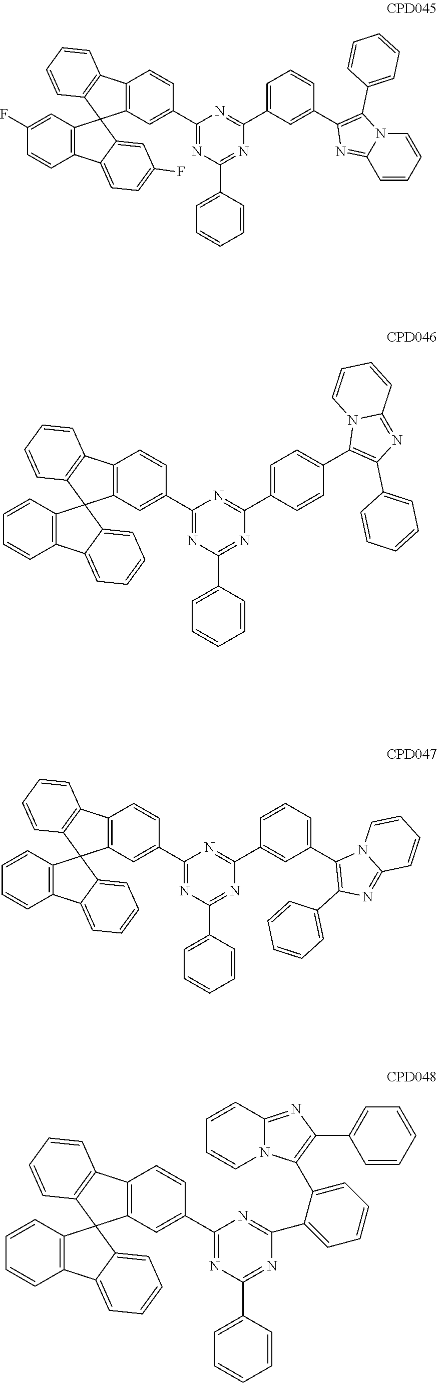 [SMILES: Fc1ccc2c(c1)C1(c3ccccc3-c3ccc(-c4nc(-c5ccccc5)nc(-c5cccc(-c6nc7ccccn7c6-c6ccccc6)c5)n4)cc31)c1cc(F)ccc1-2 ; c1ccc(-c2nc(-c3ccc(-c4c(-c5ccccc5)nc5ccccn45)cc3)nc(-c3ccc4c(c3)C3(c5ccccc5-c5ccccc53)c3ccccc3-4)n2)cc1 ; c1ccc(-c2nc(-c3cccc(-c4c(-c5ccccc5)nc5ccccn45)c3)nc(-c3ccc4c(c3)C3(c5ccccc5-c5ccccc53)c3ccccc3-4)n2)cc1 ; c1ccc(-c2nc(-c3ccc4c(c3)C3(c5ccccc5-c5ccccc53)c3ccccc3-4)nc(-c3ccccc3-c3c(-c4ccccc4)nc4ccccn34)n2)cc1]

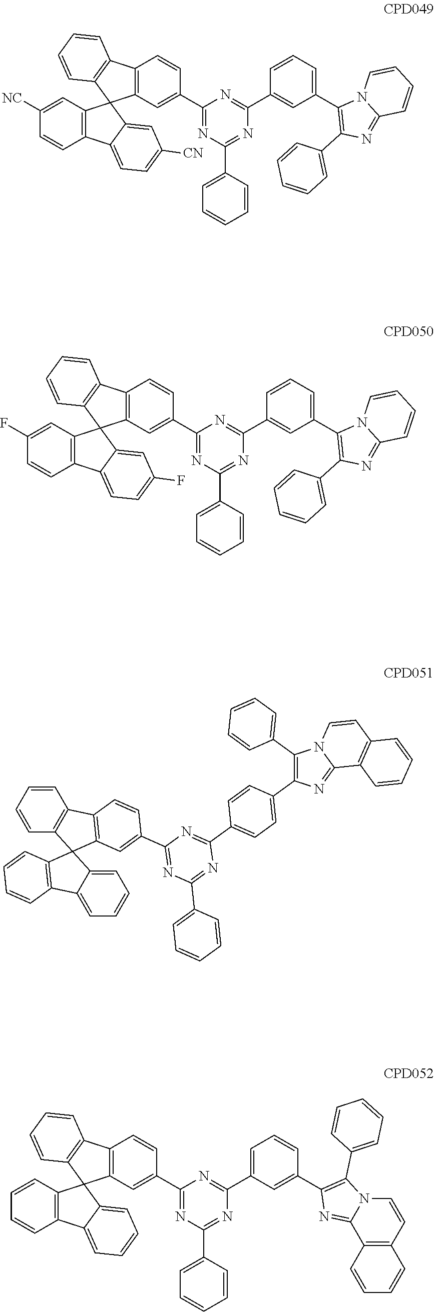 [SMILES: N#Cc1ccc2c(c1)C1(c3ccccc3-c3ccc(-c4nc(-c5ccccc5)nc(-c5cccc(-c6c(-c7ccccc7)nc7ccccn67)c5)n4)cc31)c1cc(C#N)ccc1-2 ; Fc1ccc2c(c1)C1(c3ccccc3-c3ccc(-c4nc(-c5ccccc5)nc(-c5cccc(-c6c(-c7ccccc7)nc7ccccn67)c5)n4)cc31)c1cc(F)ccc1-2 ; c1ccc(-c2nc(-c3ccc(-c4nc5c6ccccc6ccn5c4-c4ccccc4)cc3)nc(-c3ccc4c(c3)C3(c5ccccc5-c5ccccc53)c3ccccc3-4)n2)cc1 ; c1ccc(-c2nc(-c3cccc(-c4nc5c6ccccc6ccn5c4-c4ccccc4)c3)nc(-c3ccc4c(c3)C3(c5ccccc5-c5ccccc53)c3ccccc3-4)n2)cc1]

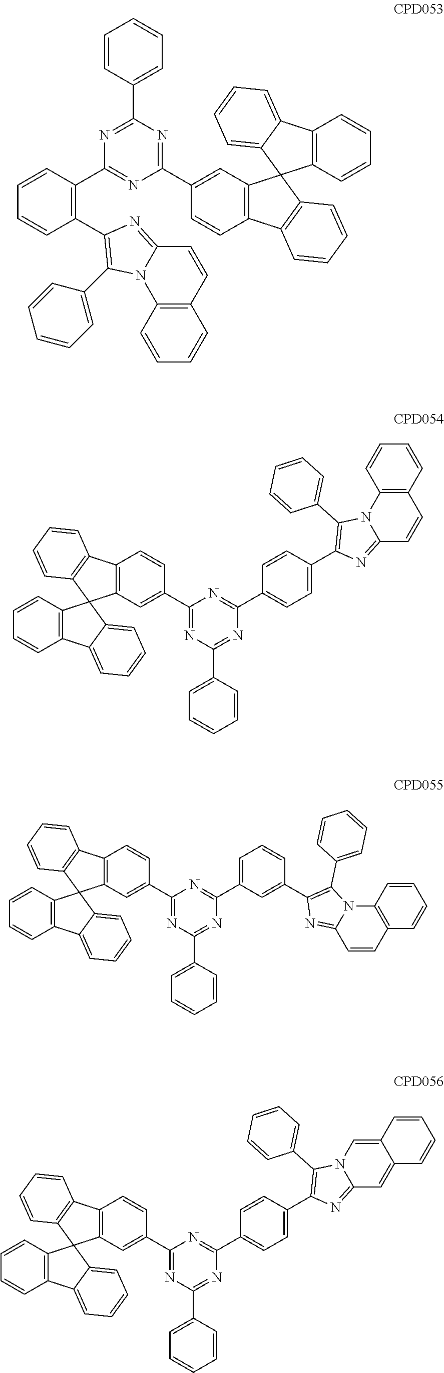 [SMILES: c1ccc(-c2nc(-c3ccc4c(c3)C3(c5ccccc5-c5ccccc53)c3ccccc3-4)nc(-c3ccccc3-c3nc4ccc5ccccc5n4c3-c3ccccc3)n2)cc1 ; c1ccc(-c2nc(-c3ccc(-c4nc5ccc6ccccc6n5c4-c4ccccc4)cc3)nc(-c3ccc4c(c3)C3(c5ccccc5-c5ccccc53)c3ccccc3-4)n2)cc1 ; c1ccc(-c2nc(-c3cccc(-c4nc5ccc6ccccc6n5c4-c4ccccc4)c3)nc(-c3ccc4c(c3)C3(c5ccccc5-c5ccccc53)c3ccccc3-4)n2)cc1 ; c1ccc(-c2nc(-c3ccc(-c4nc5cc6ccccc6cn5c4-c4ccccc4)cc3)nc(-c3ccc4c(c3)C3(c5ccccc5-c5ccccc53)c3ccccc3-4)n2)cc1]

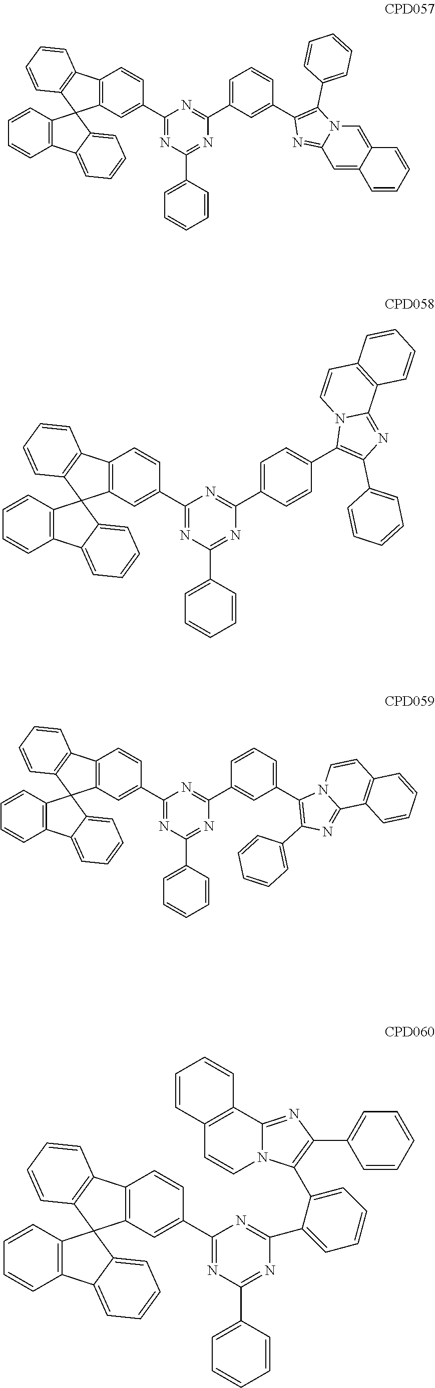 [SMILES: c1ccc(-c2nc(-c3cccc(-c4nc5cc6ccccc6cn5c4-c4ccccc4)c3)nc(-c3ccc4c(c3)C3(c5ccccc5-c5ccccc53)c3ccccc3-4)n2)cc1 ; c1ccc(-c2nc(-c3ccc(-c4c(-c5ccccc5)nc5c6ccccc6ccn45)cc3)nc(-c3ccc4c(c3)C3(c5ccccc5-c5ccccc53)c3ccccc3-4)n2)cc1 ; c1ccc(-c2nc(-c3cccc(-c4c(-c5ccccc5)nc5c6ccccc6ccn45)c3)nc(-c3ccc4c(c3)C3(c5ccccc5-c5ccccc53)c3ccccc3-4)n2)cc1 ; c1ccc(-c2nc(-c3ccc4c(c3)C3(c5ccccc5-c5ccccc53)c3ccccc3-4)nc(-c3ccccc3-c3c(-c4ccccc4)nc4c5ccccc5ccn34)n2)cc1]

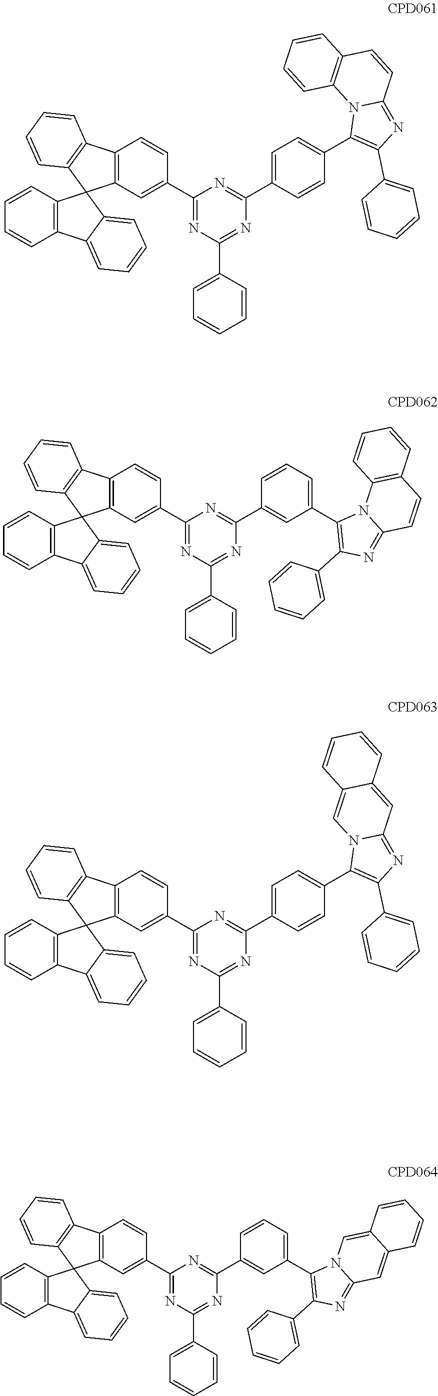 [SMILES: c1ccc(-c2nc(-c3ccc(-c4c(-c5ccccc5)nc5ccc6ccccc6n45)cc3)nc(-c3ccc4c(c3)C3(c5ccccc5-c5ccccc53)c3ccccc3-4)n2)cc1 ; c1ccc(-c2nc(-c3cccc(-c4c(-c5ccccc5)nc5ccc6ccccc6n45)c3)nc(-c3ccc4c(c3)C3(c5ccccc5-c5ccccc53)c3ccccc3-4)n2)cc1 ; c1ccc(-c2nc(-c3ccc(-c4c(-c5ccccc5)nc5cc6ccccc6cn45)cc3)nc(-c3ccc4c(c3)C3(c5ccccc5-c5ccccc53)c3ccccc3-4)n2)cc1 ; c1ccc(-c2nc(-c3cccc(-c4c(-c5ccccc5)nc5cc6ccccc6cn45)c3)nc(-c3ccc4c(c3)C3(c5ccccc5-c5ccccc53)c3ccccc3-4)n2)cc1]

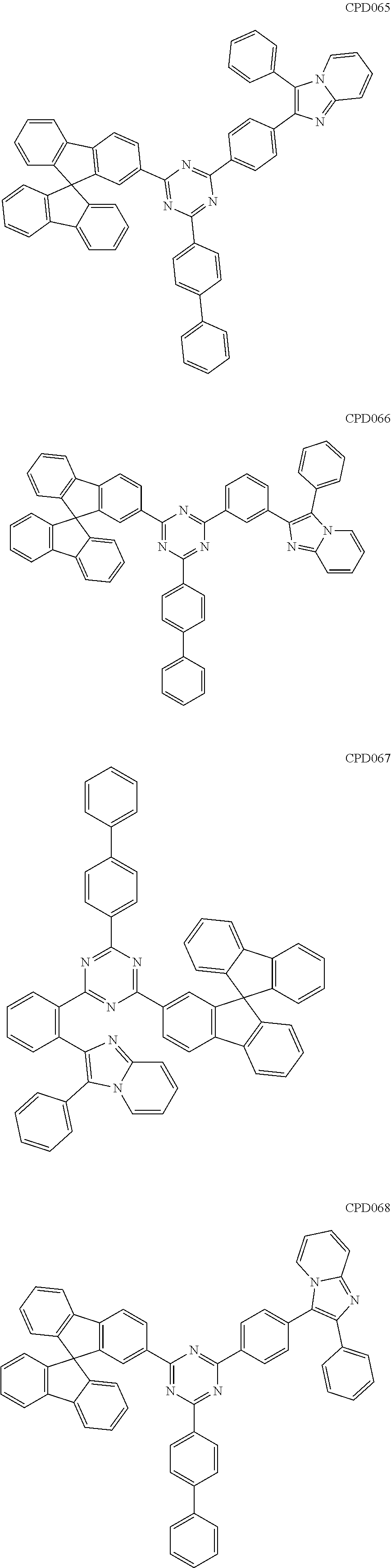 [SMILES: c1ccc(-c2ccc(-c3nc(-c4ccc(-c5nc6ccccn6c5-c5ccccc5)cc4)nc(-c4ccc5c(c4)C4(c6ccccc6-c6ccccc64)c4ccccc4-5)n3)cc2)cc1 ; c1ccc(-c2ccc(-c3nc(-c4cccc(-c5nc6ccccn6c5-c5ccccc5)c4)nc(-c4ccc5c(c4)C4(c6ccccc6-c6ccccc64)c4ccccc4-5)n3)cc2)cc1 ; c1ccc(-c2ccc(-c3nc(-c4ccc5c(c4)C4(c6ccccc6-c6ccccc64)c4ccccc4-5)nc(-c4ccccc4-c4nc5ccccn5c4-c4ccccc4)n3)cc2)cc1 ; c1ccc(-c2ccc(-c3nc(-c4ccc(-c5c(-c6ccccc6)nc6ccccn56)cc4)nc(-c4ccc5c(c4)C4(c6ccccc6-c6ccccc64)c4ccccc4-5)n3)cc2)cc1]

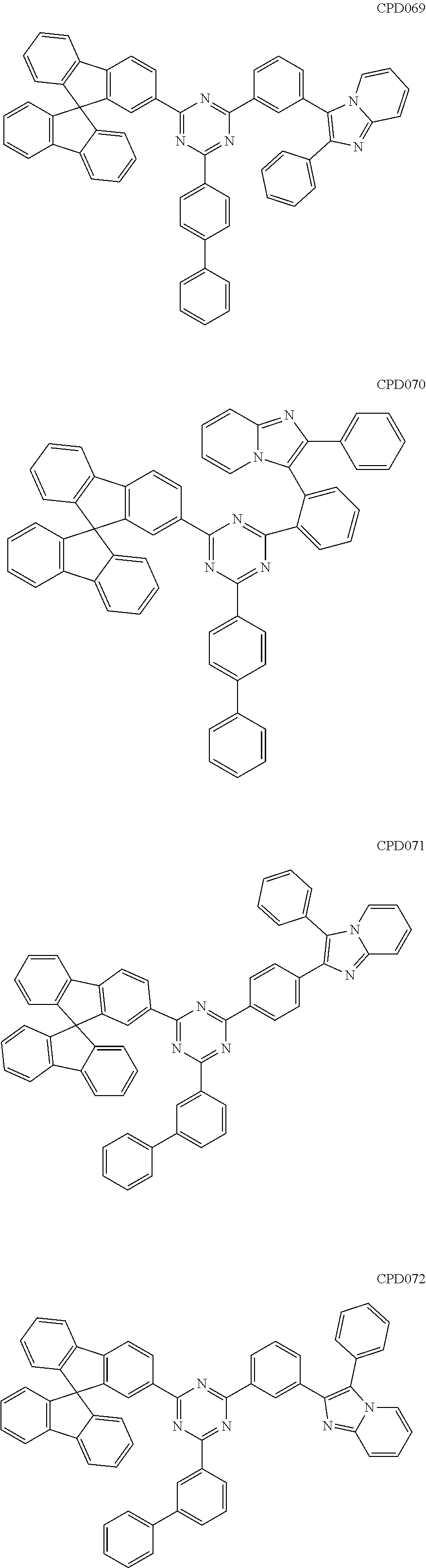 [SMILES: c1ccc(-c2ccc(-c3nc(-c4cccc(-c5c(-c6ccccc6)nc6ccccn56)c4)nc(-c4ccc5c(c4)C4(c6ccccc6-c6ccccc64)c4ccccc4-5)n3)cc2)cc1 ; c1ccc(-c2ccc(-c3nc(-c4ccc5c(c4)C4(c6ccccc6-c6ccccc64)c4ccccc4-5)nc(-c4ccccc4-c4c(-c5ccccc5)nc5ccccn45)n3)cc2)cc1 ; c1ccc(-c2cccc(-c3nc(-c4ccc(-c5nc6ccccn6c5-c5ccccc5)cc4)nc(-c4ccc5c(c4)C4(c6ccccc6-c6ccccc64)c4ccccc4-5)n3)c2)cc1 ; c1ccc(-c2cccc(-c3nc(-c4cccc(-c5nc6ccccn6c5-c5ccccc5)c4)nc(-c4ccc5c(c4)C4(c6ccccc6-c6ccccc64)c4ccccc4-5)n3)c2)cc1]

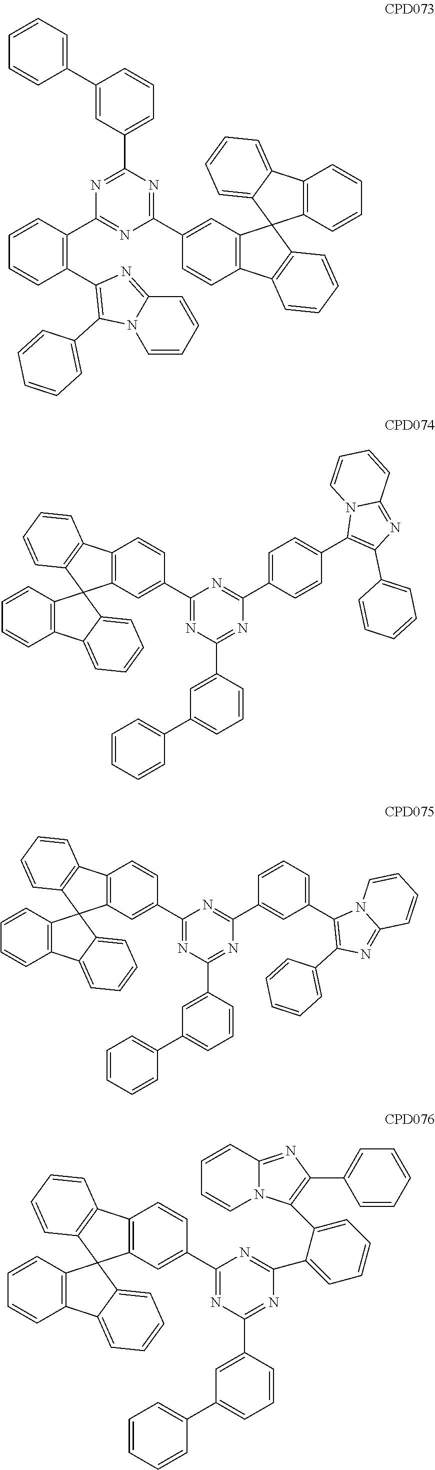 [SMILES: c1ccc(-c2cccc(-c3nc(-c4ccc5c(c4)C4(c6ccccc6-c6ccccc64)c4ccccc4-5)nc(-c4ccccc4-c4nc5ccccn5c4-c4ccccc4)n3)c2)cc1 ; c1ccc(-c2cccc(-c3nc(-c4ccc(-c5c(-c6ccccc6)nc6ccccn56)cc4)nc(-c4ccc5c(c4)C4(c6ccccc6-c6ccccc64)c4ccccc4-5)n3)c2)cc1 ; c1ccc(-c2cccc(-c3nc(-c4cccc(-c5c(-c6ccccc6)nc6ccccn56)c4)nc(-c4ccc5c(c4)C4(c6ccccc6-c6ccccc64)c4ccccc4-5)n3)c2)cc1 ; c1ccc(-c2cccc(-c3nc(-c4ccc5c(c4)C4(c6ccccc6-c6ccccc64)c4ccccc4-5)nc(-c4ccccc4-c4c(-c5ccccc5)nc5ccccn45)n3)c2)cc1]

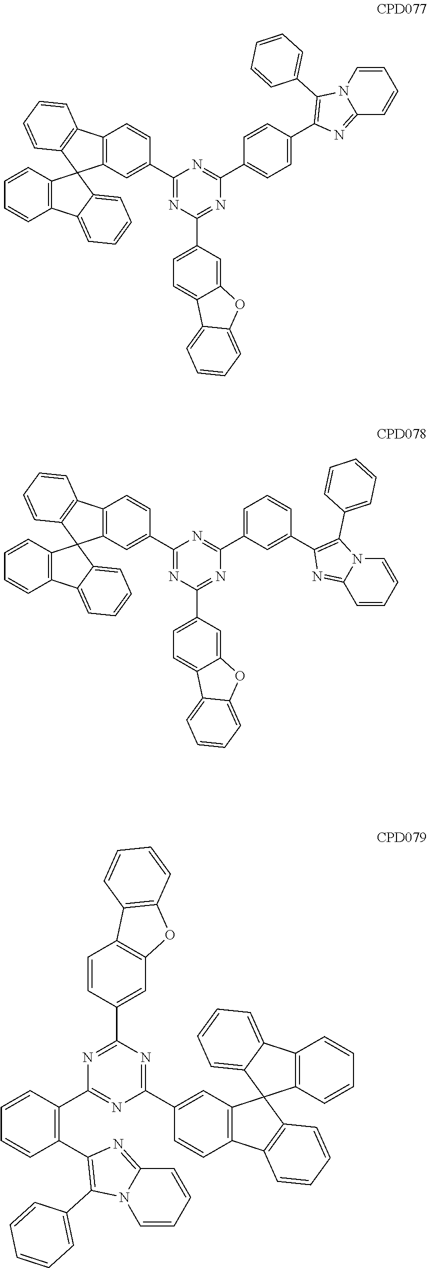 [SMILES: c1ccc(-c2c(-c3ccc(-c4nc(-c5ccc6c(c5)C5(c7ccccc7-c7ccccc75)c5ccccc5-6)nc(-c5ccc6c(c5)oc5ccccc56)n4)cc3)nc3ccccn23)cc1 ; c1ccc(-c2c(-c3cccc(-c4nc(-c5ccc6c(c5)C5(c7ccccc7-c7ccccc75)c5ccccc5-6)nc(-c5ccc6c(c5)oc5ccccc56)n4)c3)nc3ccccn23)cc1 ; c1ccc(-c2c(-c3ccccc3-c3nc(-c4ccc5c(c4)C4(c6ccccc6-c6ccccc64)c4ccccc4-5)nc(-c4ccc5c(c4)oc4ccccc45)n3)nc3ccccn23)cc1]

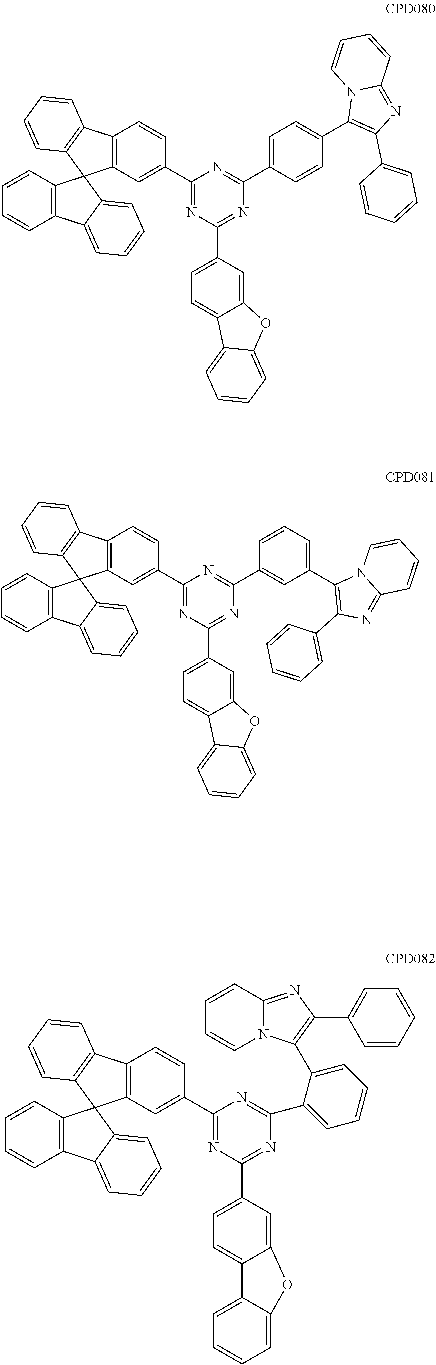 [SMILES: c1ccc(-c2nc3ccccn3c2-c2ccc(-c3nc(-c4ccc5c(c4)C4(c6ccccc6-c6ccccc64)c4ccccc4-5)nc(-c4ccc5c(c4)oc4ccccc45)n3)cc2)cc1 ; c1ccc(-c2nc3ccccn3c2-c2cccc(-c3nc(-c4ccc5c(c4)C4(c6ccccc6-c6ccccc64)c4ccccc4-5)nc(-c4ccc5c(c4)oc4ccccc45)n3)c2)cc1 ; c1ccc(-c2nc3ccccn3c2-c2ccccc2-c2nc(-c3ccc4c(c3)C3(c5ccccc5-c5ccccc53)c3ccccc3-4)nc(-c3ccc4c(c3)oc3ccccc34)n2)cc1]

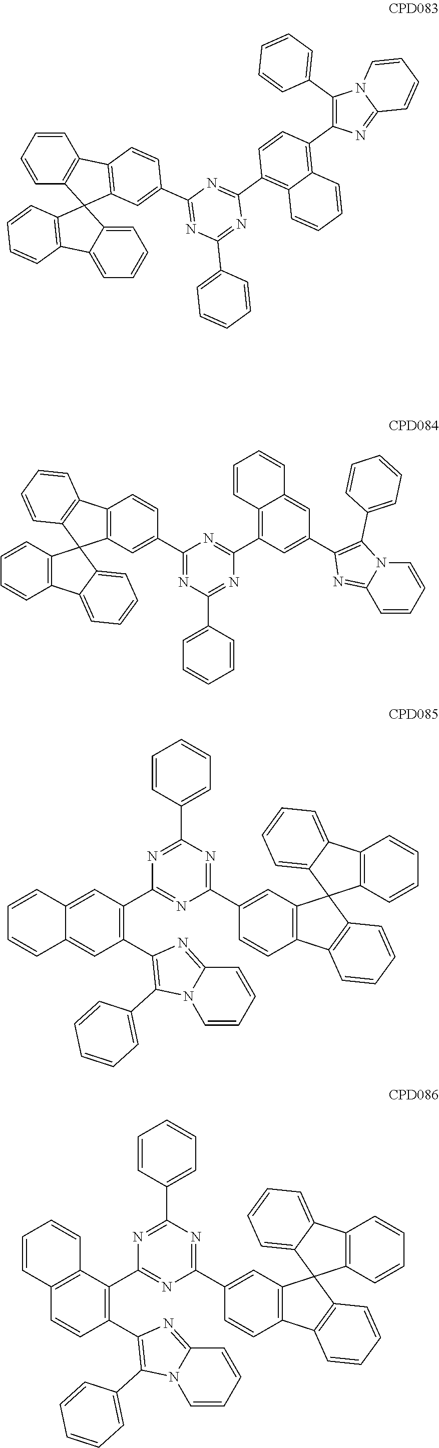 [SMILES: c1ccc(-c2nc(-c3ccc4c(c3)C3(c5ccccc5-c5ccccc53)c3ccccc3-4)nc(-c3ccc(-c4nc5ccccn5c4-c4ccccc4)c4ccccc34)n2)cc1 ; c1ccc(-c2nc(-c3ccc4c(c3)C3(c5ccccc5-c5ccccc53)c3ccccc3-4)nc(-c3cc(-c4nc5ccccn5c4-c4ccccc4)cc4ccccc34)n2)cc1 ; c1ccc(-c2nc(-c3ccc4c(c3)C3(c5ccccc5-c5ccccc53)c3ccccc3-4)nc(-c3cc4ccccc4cc3-c3nc4ccccn4c3-c3ccccc3)n2)cc1 ; c1ccc(-c2nc(-c3ccc4c(c3)C3(c5ccccc5-c5ccccc53)c3ccccc3-4)nc(-c3c(-c4nc5ccccn5c4-c4ccccc4)ccc4ccccc34)n2)cc1]

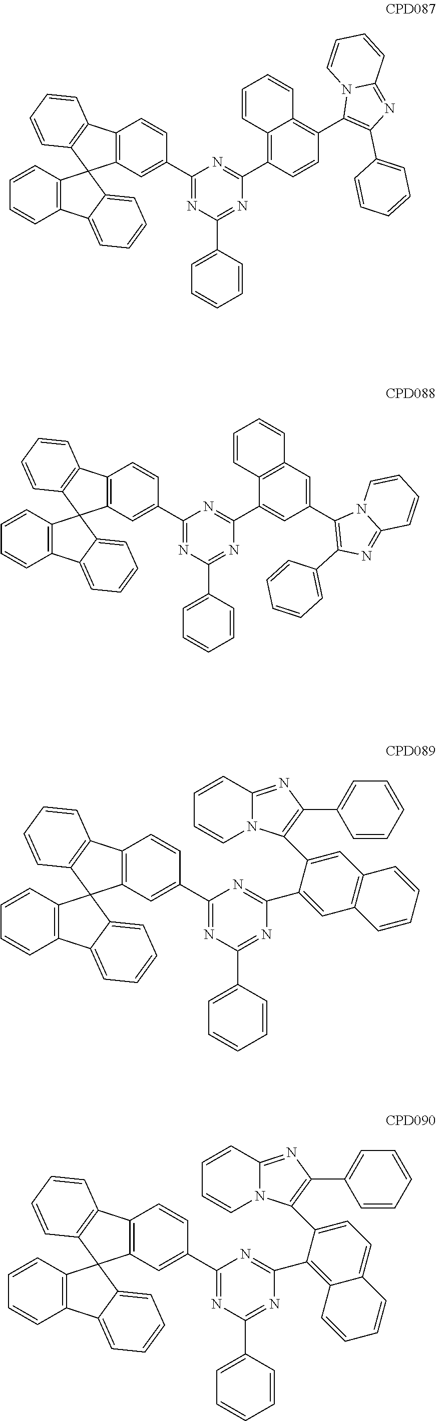 [SMILES: c1ccc(-c2nc(-c3ccc4c(c3)C3(c5ccccc5-c5ccccc53)c3ccccc3-4)nc(-c3ccc(-c4c(-c5ccccc5)nc5ccccn45)c4ccccc34)n2)cc1 ; c1ccc(-c2nc(-c3ccc4c(c3)C3(c5ccccc5-c5ccccc53)c3ccccc3-4)nc(-c3cc(-c4c(-c5ccccc5)nc5ccccn45)cc4ccccc34)n2)cc1 ; c1ccc(-c2nc(-c3ccc4c(c3)C3(c5ccccc5-c5ccccc53)c3ccccc3-4)nc(-c3cc4ccccc4cc3-c3c(-c4ccccc4)nc4ccccn34)n2)cc1 ; c1ccc(-c2nc(-c3ccc4c(c3)C3(c5ccccc5-c5ccccc53)c3ccccc3-4)nc(-c3c(-c4c(-c5ccccc5)nc5ccccn45)ccc4ccccc34)n2)cc1]

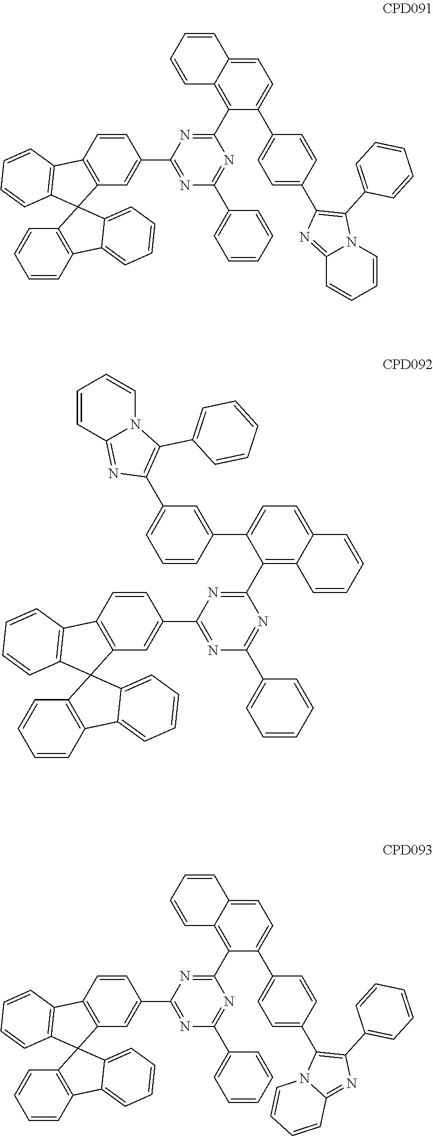 [SMILES: c1ccc(-c2nc(-c3ccc4c(c3)C3(c5ccccc5-c5ccccc53)c3ccccc3-4)nc(-c3c(-c4ccc(-c5nc6ccccn6c5-c5ccccc5)cc4)ccc4ccccc34)n2)cc1 ; c1ccc(-c2nc(-c3ccc4c(c3)C3(c5ccccc5-c5ccccc53)c3ccccc3-4)nc(-c3c(-c4cccc(-c5nc6ccccn6c5-c5ccccc5)c4)ccc4ccccc34)n2)cc1 ; c1ccc(-c2nc(-c3ccc4c(c3)C3(c5ccccc5-c5ccccc53)c3ccccc3-4)nc(-c3c(-c4ccc(-c5c(-c6ccccc6)nc6ccccn56)cc4)ccc4ccccc34)n2)cc1]

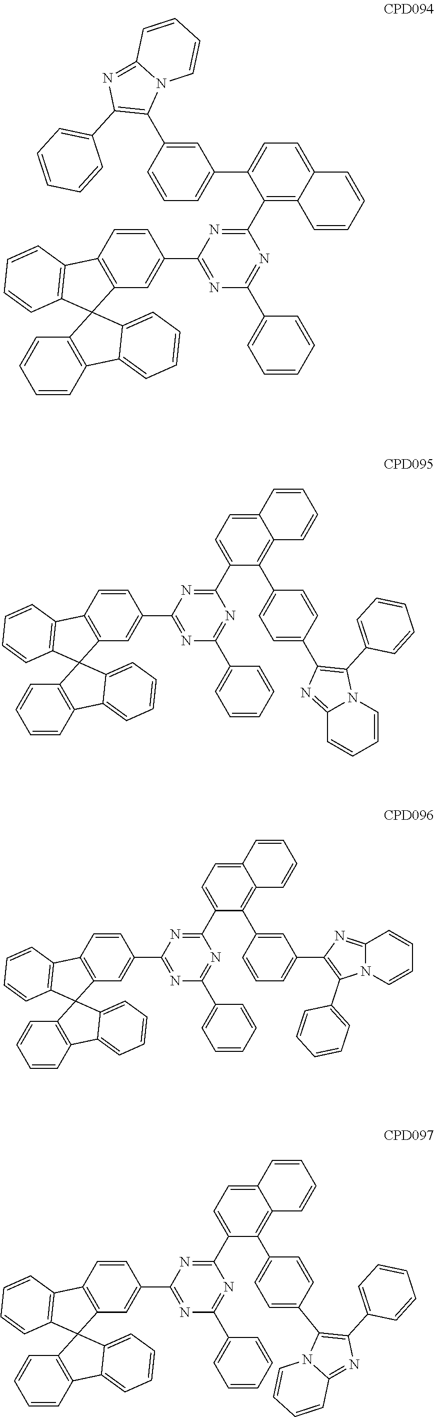 [SMILES: c1ccc(-c2nc(-c3ccc4c(c3)C3(c5ccccc5-c5ccccc53)c3ccccc3-4)nc(-c3c(-c4cccc(-c5c(-c6ccccc6)nc6ccccn56)c4)ccc4ccccc34)n2)cc1 ; c1ccc(-c2nc(-c3ccc4c(c3)C3(c5ccccc5-c5ccccc53)c3ccccc3-4)nc(-c3ccc4ccccc4c3-c3ccc(-c4nc5ccccn5c4-c4ccccc4)cc3)n2)cc1 ; c1ccc(-c2nc(-c3ccc4c(c3)C3(c5ccccc5-c5ccccc53)c3ccccc3-4)nc(-c3ccc4ccccc4c3-c3cccc(-c4nc5ccccn5c4-c4ccccc4)c3)n2)cc1 ; c1ccc(-c2nc(-c3ccc4c(c3)C3(c5ccccc5-c5ccccc53)c3ccccc3-4)nc(-c3ccc4ccccc4c3-c3ccc(-c4c(-c5ccccc5)nc5ccccn45)cc3)n2)cc1]

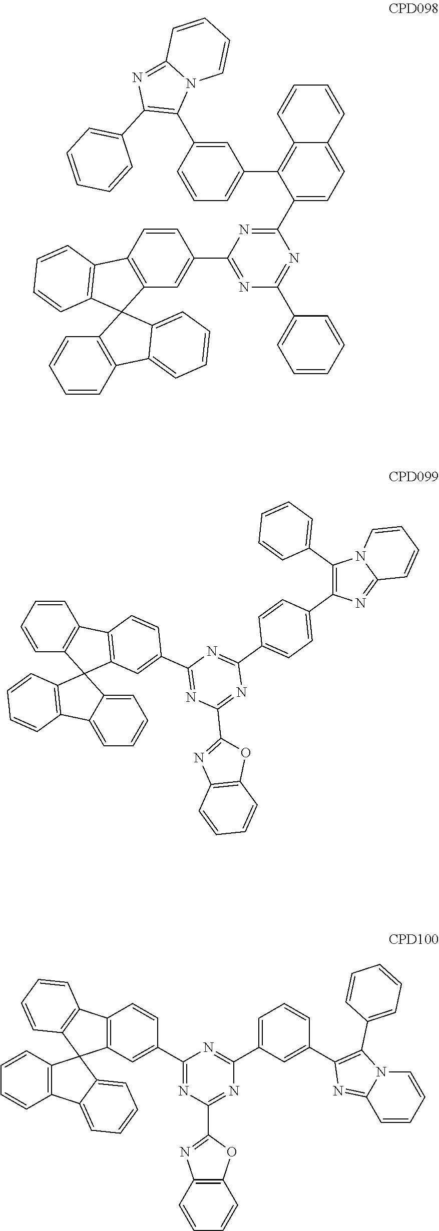 [SMILES: c1ccc(-c2nc(-c3ccc4c(c3)C3(c5ccccc5-c5ccccc53)c3ccccc3-4)nc(-c3ccc4ccccc4c3-c3cccc(-c4c(-c5ccccc5)nc5ccccn45)c3)n2)cc1 ; c1ccc(-c2c(-c3ccc(-c4nc(-c5ccc6c(c5)C5(c7ccccc7-c7ccccc75)c5ccccc5-6)nc(-c5nc6ccccc6o5)n4)cc3)nc3ccccn23)cc1 ; c1ccc(-c2c(-c3cccc(-c4nc(-c5ccc6c(c5)C5(c7ccccc7-c7ccccc75)c5ccccc5-6)nc(-c5nc6ccccc6o5)n4)c3)nc3ccccn23)cc1]

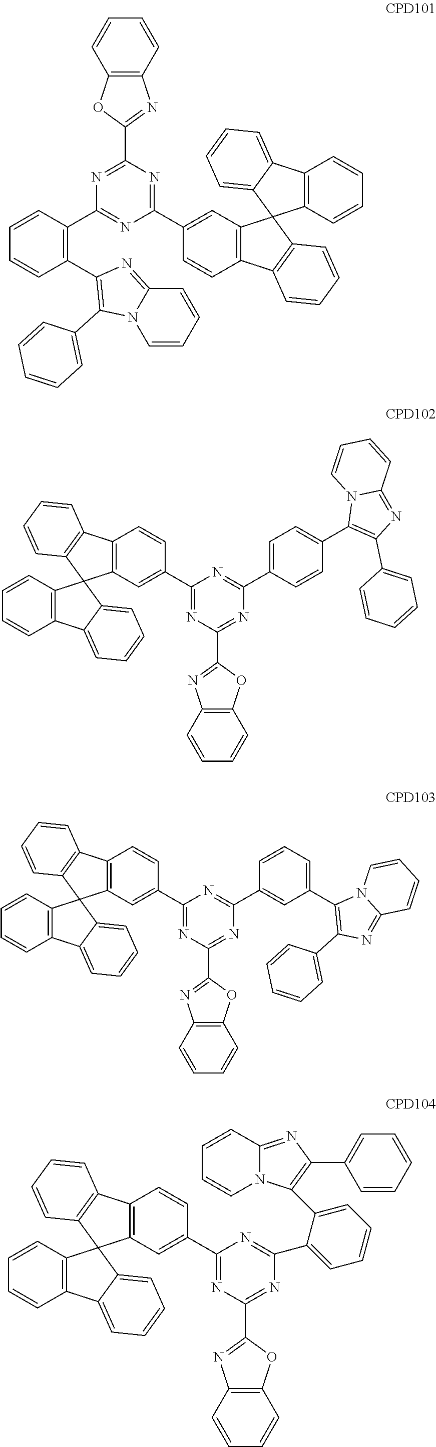 [SMILES: c1ccc(-c2c(-c3ccccc3-c3nc(-c4ccc5c(c4)C4(c6ccccc6-c6ccccc64)c4ccccc4-5)nc(-c4nc5ccccc5o4)n3)nc3ccccn23)cc1 ; c1ccc(-c2nc3ccccn3c2-c2ccc(-c3nc(-c4ccc5c(c4)C4(c6ccccc6-c6ccccc64)c4ccccc4-5)nc(-c4nc5ccccc5o4)n3)cc2)cc1 ; c1ccc(-c2nc3ccccn3c2-c2cccc(-c3nc(-c4ccc5c(c4)C4(c6ccccc6-c6ccccc64)c4ccccc4-5)nc(-c4nc5ccccc5o4)n3)c2)cc1 ; c1ccc(-c2nc3ccccn3c2-c2ccccc2-c2nc(-c3ccc4c(c3)C3(c5ccccc5-c5ccccc53)c3ccccc3-4)nc(-c3nc4ccccc4o3)n2)cc1]

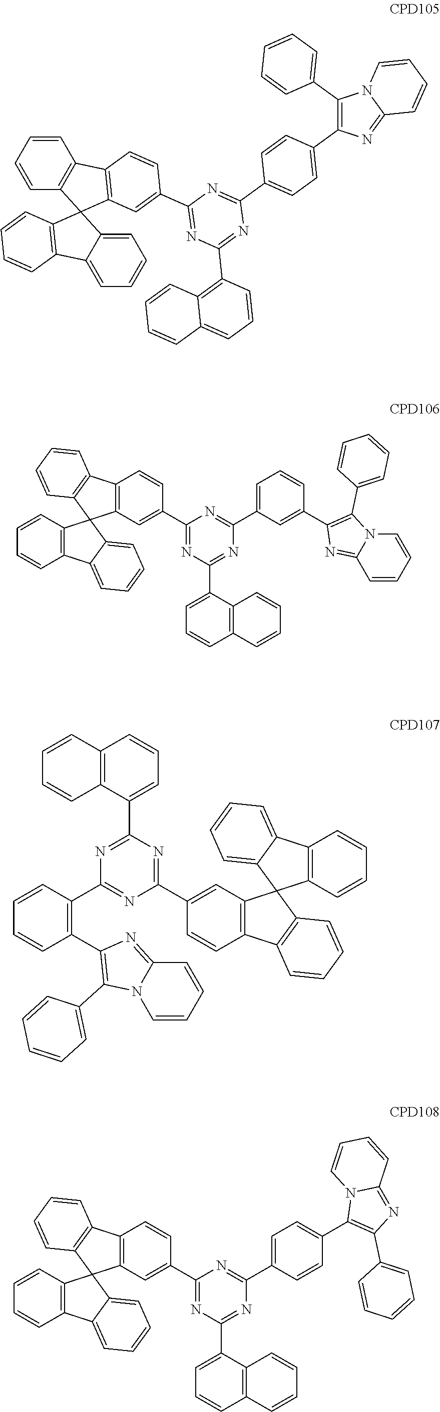 [SMILES: c1ccc(-c2c(-c3ccc(-c4nc(-c5ccc6c(c5)C5(c7ccccc7-c7ccccc75)c5ccccc5-6)nc(-c5cccc6ccccc56)n4)cc3)nc3ccccn23)cc1 ; c1ccc(-c2c(-c3cccc(-c4nc(-c5ccc6c(c5)C5(c7ccccc7-c7ccccc75)c5ccccc5-6)nc(-c5cccc6ccccc56)n4)c3)nc3ccccn23)cc1 ; c1ccc(-c2c(-c3ccccc3-c3nc(-c4ccc5c(c4)C4(c6ccccc6-c6ccccc64)c4ccccc4-5)nc(-c4cccc5ccccc45)n3)nc3ccccn23)cc1 ; c1ccc(-c2nc3ccccn3c2-c2ccc(-c3nc(-c4ccc5c(c4)C4(c6ccccc6-c6ccccc64)c4ccccc4-5)nc(-c4cccc5ccccc45)n3)cc2)cc1]

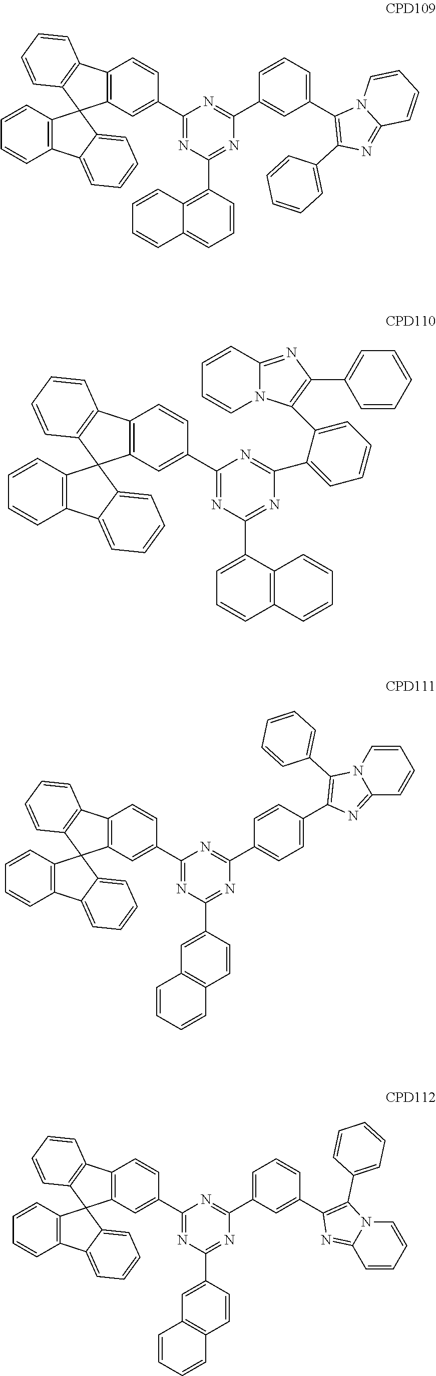 [SMILES: c1ccc(-c2nc3ccccn3c2-c2cccc(-c3nc(-c4ccc5c(c4)C4(c6ccccc6-c6ccccc64)c4ccccc4-5)nc(-c4cccc5ccccc45)n3)c2)cc1 ; c1ccc(-c2nc3ccccn3c2-c2ccccc2-c2nc(-c3ccc4c(c3)C3(c5ccccc5-c5ccccc53)c3ccccc3-4)nc(-c3cccc4ccccc34)n2)cc1 ; c1ccc(-c2c(-c3ccc(-c4nc(-c5ccc6c(c5)C5(c7ccccc7-c7ccccc75)c5ccccc5-6)nc(-c5ccc6ccccc6c5)n4)cc3)nc3ccccn23)cc1 ; c1ccc(-c2c(-c3cccc(-c4nc(-c5ccc6c(c5)C5(c7ccccc7-c7ccccc75)c5ccccc5-6)nc(-c5ccc6ccccc6c5)n4)c3)nc3ccccn23)cc1]

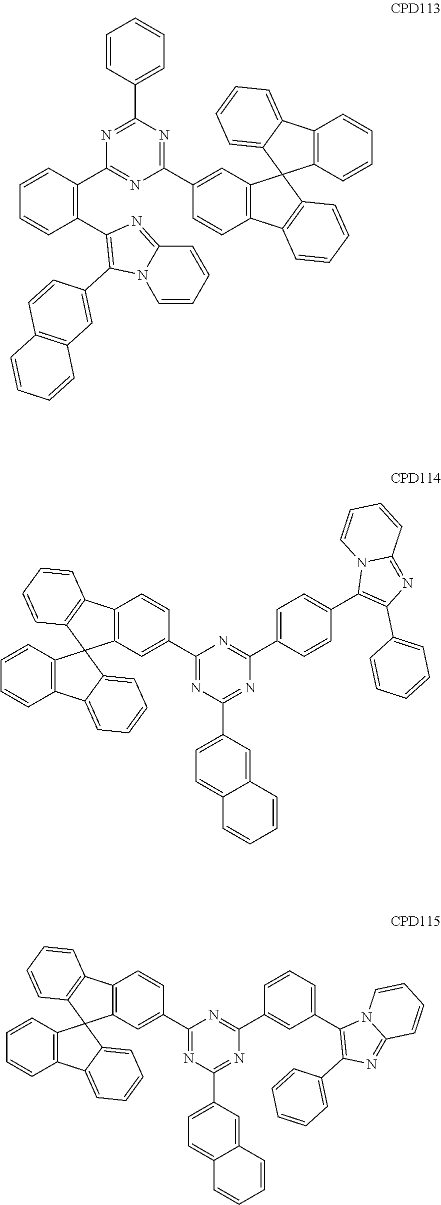 [SMILES: c1ccc(-c2nc(-c3ccc4c(c3)C3(c5ccccc5-c5ccccc53)c3ccccc3-4)nc(-c3ccccc3-c3nc4ccccn4c3-c3ccc4ccccc4c3)n2)cc1 ; c1ccc(-c2nc3ccccn3c2-c2ccc(-c3nc(-c4ccc5c(c4)C4(c6ccccc6-c6ccccc64)c4ccccc4-5)nc(-c4ccc5ccccc5c4)n3)cc2)cc1 ; c1ccc(-c2nc3ccccn3c2-c2cccc(-c3nc(-c4ccc5c(c4)C4(c6ccccc6-c6ccccc64)c4ccccc4-5)nc(-c4ccc5ccccc5c4)n3)c2)cc1]

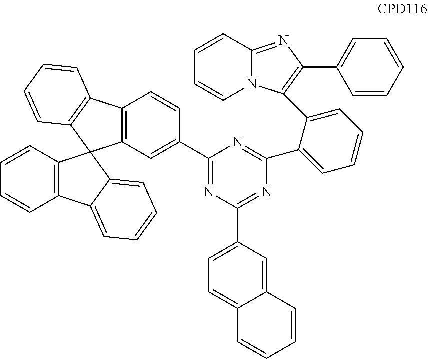 [SMILES: c1ccc(-c2nc3ccccn3c2-c2ccccc2-c2nc(-c3ccc4c(c3)C3(c5ccccc5-c5ccccc53)c3ccccc3-4)nc(-c3ccc4ccccc4c3)n2)cc1]

17. (canceled)

18. (Canceled)

19. An organic electroluminescent device comprising a spirocyclic compound as claimed in claim 1.

20. The organic electroluminescent device according to claim 19, comprising an electron injection layer or an electron transport layer, wherein the material of the electron injection layer or electron transport layer comprises a spirocyclic compound as claimed in claim 1.
